SEQUENCE LISTING (17 sequences)

SEQ ID NO: 1 - DNA, 20 bp, synthetic construct
  source 1..20: mol_type = other DNA
aggacagtcagacagtcatg

SEQ ID NO: 2 - DNA, 20 bp, synthetic construct
  source 1..20: mol_type = other DNA
aggtcagacagtcagacaca

SEQ ID NO: 3 - DNA, 20 bp, synthetic construct
  source 1..20: mol_type = other DNA
aggtcagtcagtaagtaacg

SEQ ID NO: 4 - DNA, 23 bp, synthetic construct
  source 1..23: mol_type = other DNA
gtgcaatcctgtcggaatgcagg

SEQ ID NO: 5 - DNA, 12 bp, synthetic construct
  source 1..12: mol_type = other DNA
aggaggaaaaaa

SEQ ID NO: 6 - DNA, 12 bp, synthetic construct
  source 1..12: mol_type = other DNA
gggaagaaggaa

SEQ ID NO: 7 - DNA, 12 bp, synthetic construct
  source 1..12: mol_type = other DNA
ggaaagaaagaa

SEQ ID NO: 8 - DNA, 12 bp, synthetic construct
  source 1..12: mol_type = other DNA
gagaaagagaag

SEQ ID NO: 9 - DNA, 12 bp, synthetic construct
  source 1..12: mol_type = other DNA
aggaggaaaaag

SEQ ID NO: 10 - DNA, 40 bp, synthetic construct
  source 1..40: mol_type = other DNA
nggnnagnnagnnagnnannnggnnagnnagnnagnnann

SEQ ID NO: 11 - DNA, 16 bp, synthetic construct
  source 1..16: mol_type = other DNA
ggggagggaggggagg

SEQ ID NO: 12 - DNA, 15 bp, synthetic construct
  source 1..15: mol_type = other DNA
gggaaggaagggaag

SEQ ID NO: 13 - DNA, 14 bp, synthetic construct
  source 1..14: mol_type = other DNA
ggaaagaaaggaaa

SEQ ID NO: 14 - DNA, 13 bp, synthetic construct
  source 1..13: mol_type = other DNA
gaaaaaaaagaaa

SEQ ID NO: 15 - DNA, 11 bp, synthetic construct
  source 1..11: mol_type = other DNA
aggacagtcag

SEQ ID NO: 16 - DNA, 12 bp, synthetic construct
  source 1..12: mol_type = other DNA
aaaaaaaaaaaa

SEQ ID NO: 17 - DNA, 80 bp, synthetic construct
  source 1..80: mol_type = other DNA
tgtagtgaagagaacgacccgatgttaccaccccaagagagatacaactccaactgttccccagttaccatcccaagcct

(19) United States
(12) Patent Application Publication
(10) Pub. No.: US 2025/0075202 A1
(43) Pub. Date: Mar. 6, 2025

(54) MOLECULAR RECORDING METHODS AND SYSTEMS TO CAPTURE LINEAGE RELATIONSHIPS IN DIFFERENTIATING STEM CELLS

(71) Applicant: California Institute of Technology, Pasadena, CA (US)

(72) Inventors: Duncan M. Chadly, Pasadena, CA (US); Michael B. Elowitz, Pasadena, CA (US)

(21) Appl. No.: 18/799,870

(22) Filed: Aug. 9, 2024

Related U.S. Application Data
(60) Provisional application No. 63/531,874, filed on Aug. 10, 2023.

(51) Int. Cl. C12N 15/11; C12N 9/22; C12N 15/90; C12Q 1/6841

(52) U.S. Cl. CPC C12N 15/11; C12N 9/22; C12N 15/907; C12Q 1/6841; C12N 2310/20; C12N 2830/002

(57) ABSTRACT

Disclosed herein include methods, compositions, and kits suitable for use in capturing lineage relationships in dividing cell populations. Disclosed herein include compact phylogenetic recording systems for high resolution lineage reconstruction over long time scales. In some embodiments, the system comprises one or more hypercascade array(s) each comprising p hypercascade units, n layer guide RNAs, and an editor. In some embodiments, the system comprises one or more target array(s) comprising n editable target sites, n guide RNAs gRNAs, and a base editor capable of adenine (A)-to-guanine (G) base editing.

### RELATED APPLICATIONS

This application claims the benefit under 35 U.S.C. § 119(e) of U.S. Provisional Patent Application Ser. No. 63/531,874, filed Aug. 10, 2023, the content of this related application is incorporated herein by reference in its entirety for all purposes.

### STATEMENT REGARDING FEDERALLY SPONSORED R&D

This invention was made with government support under Grant No. MH116508 awarded by the National Institutes of Health. The government has certain rights in the invention.

### REFERENCE TO SEQUENCE LISTING

The present application is being filed along with a Sequence Listing in electronic format. The Sequence Listing is provided as a file entitled 30KJ-365874-US, created Aug. 8, 2024, which is 15,850 bytes in size. The information in the electronic format of the Sequence Listing is incorporated herein by reference in its entirety.

### BACKGROUND

### Field

The present disclosure relates generally to the field of molecular recording systems.

### Description of the Related Art

Cells divide, differentiate, and move to form exquisitely organized structures. Reconstructing the dynamic histories of individual cells, particularly their lineage relationships, could enable researchers to understand how tissues form, analyze the roles of intrinsic and extrinsic determinants of cell fate decisions, and reveal how processes are dysregulated in disease. Recent advances in single cell sequencing and spatial genomics now allow capturing single cell states at specific moments in time. However, with a few exceptions, the histories of those cells have largely remained hidden. Engineering cells to actively record information within their own genomic DNA could reveal these histories, but existing recording systems have limited information capacity or disrupt spatial context. There is a need for compact phylogenetic recording systems for high resolution lineage reconstruction over long time scales.

### SUMMARY

Disclosed herein include systems. In some embodiments, the system comprises: (i) one or more hypercascade array(s) each comprising p hypercascade units, wherein p is an integer greater than 1; (ii) n layer guide RNAs (gRNAs), wherein n is an integer greater than 1; and/or (iii) an editor. In some embodiments, the editor is a base editor capable of base editing the hypercascade array. In some embodiments, said base editing comprises: adenine (A)-to-guanine (G) base editing and/or cytosine (C)-to-thymine (T) base editing. In some embodiments, each hypercascade unit comprises m target sites, and wherein m is an integer greater than 1. In some embodiments, the target sites comprises one or more primary target site(s) and one or more conditional target site(s). In some embodiments, the target sites each comprise an editable base. In some embodiments, the editor associated with a layer gRNA is capable of editing said editable base of a target site comprising a complementary protospacer and a protospacer adjacent motif (PAM). In some embodiments, the primary target sites are capable of being edited by a first layer gRNA and the editor. In some embodiments, each of the one or more conditional target sites comprise two or more mismatches in the protospacer and/or the PAM. In some embodiments, the editing of adjacent editable bases by a previous layer gRNA are capable of repairing said mismatches, thereby enabling the editing of a conditional target site by a layer gRNA and the editor.

In some embodiments, the one or more hypercascade array(s) comprise at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 hypercascade arrays. In some embodiments, each hypercascade unit comprises the same length and/or sequence. In some embodiments, each hypercascade unit comprises an identical N-mer. In some embodiments, the N-mer is 15, 16, 17, 18, 19, 20, 21, 22, 23, 24, 25, 26, 27, 28, 29, 30, 31, 32, 33, 34, 35, 36, 37, 38, 39, or 40, nucleotides (nt) in length. In some embodiments, the N-mer is 20 nt in length. In some embodiments, the two or more of the p hypercascade units are in tandem. In some embodiments, the hypercascade array comprises a tandem repeating 20-mer. In some embodiments, the hypercascade unit comprises a nucleotide sequence that is at least 80%, 85%, 90%, 95%, 98%, 99%, or 100% identical to (NGGNNAGNNAGNNAGNNANN). In some embodiments, the hypercascade unit comprises a nucleotide sequence that is at least 80%, 85%, 90%, 95%, 98%, 99%, or 100% identical to SEQ ID NO: 1 (AGGACAGTCAGACAGTCATG). In some embodiments, the hypercascade unit comprises a nucleotide sequence that is at least 80%, 85%, 90%, 95%, 98%, 99%, or 100% identical to SEQ ID NO: 2 (AGGTCAGACAGTCAGACACA). In some embodiments, the hypercascade unit comprises a nucleotide sequence that is at least 80%, 85%, 90%, 95%, 98%, 99%, or 100% identical to SEQ ID NO: 3 (AGGTCAGTCAGTAAGTAACG). In some embodiments, the PAM is about 2-6 nucleotides in length (e.g., 3 nucleotides in length). In some embodiments, the PAM comprises NGG.

In some embodiments, each hypercascade unit comprises m−1 conditional target sites capable of being activated by adjacent edits mediated by an upper layer gRNA. In some embodiments, the editable base is situated at the fifth or sixth position of a target site. In some embodiments, the editable base comprises adenine, optionally edited to guanine by the editor. In some embodiments, the repair of repairing protospacer and PAM mismatches through A-to-G edits of a previous layer gRNA enable the editing of conditional target sites. In some embodiments, the n layer gRNAs comprise: a first layer gRNA; a second layer gRNA; a third layer gRNA; and/or a fourth layer gRNA. In some embodiments, the first layer gRNA associated with the editor is capable of editing the primary target sites. In some embodiments, the second layer gRNA associated with the editor is capable of editing first conditional target sites upon repair of adjacent mismatches by the first layer gRNA associated with the editor. In some embodiments, the third layer gRNA associated with the editor is capable of editing second conditional target sites upon repair of adjacent mismatches by the second layer gRNA associated with the editor. In some embodiments, the fourth layer gRNA associated with the editor is capable of editing third conditional target sites upon repair of adjacent mismatches by the third layer gRNA associated with the editor. In some embodiments, the first layer gRNA comprises a nucleotide sequence that is at least 80%, 85%, 90%, 95%, 98%, 99%, or 100% identical to (NGGNNAGNNAGNNAGNNANN). In some embodiments, the second layer gRNA comprises a nucleotide sequence that is at least 80%, 85%, 90%, 95%, 98%, 99%, or 100% identical to (NGGNNAGNNAGNNANNNGGN). In some embodiments, the third layer gRNA comprises a nucleotide sequence that is at least 80%, 85%, 90%, 95%, 98%, 99%, or 100% identical to (NGGNNAGNNANNNGGNNGGN). In some embodiments, the fourth layer gRNA comprises a nucleotide sequence that is at least 80%, 85%, 90%, 95%, 98%, 99%, or 100% identical to (NGGNNANNNGGNNGGNNGGN). In some embodiments, the layer gRNA is a single guide RNA (sgRNA). In some embodiments, each hypercascade array comprises: a first static barcode; and/or one or more second static barcodes (e.g., two second static barcodes). In some embodiments, the first static barcode is about 10 nt in length. In some embodiments, the first static barcode is selected from a library of at least about 10⁶ different first static barcode sequences. In some embodiments, the one or more second static barcodes are image-readable. In some embodiments, a second static barcode is selected from a library of at least about 200 different second static barcode sequences.

In some embodiments, upon introduction into a cell, the system achieves extended durations of editing with near-linear barcode loss over a period of time (e.g., at least about 2, 3, 4, 5, 6, 7, 8, 9, 10, 11, 12, 13, 14, 15, 16, 17, 18, 19, or 20, generations). In some embodiments, the relative insensitivity of the system to edit rate and hypercascade unit copy number enables deep lineage reconstruction (e.g., in polyclonal populations of cells). In some embodiments, the editor is selected from the group comprising CRISPR-Cas9, base editors, prime editors, integrases, and recombinases. In some embodiments, the editor is a base editor is capable of base editing the hypercascade unit. In some embodiments, said base editing comprises: adenine (A)-to-guanine (G) base editing and/or cytosine (C)-to-thymine (T) base editing. In some embodiments, the base editor comprises saCas9-KKH, Cas9-VQR, Cas9-VRQR, Cas9-VRER, Cas9-NG, ABE7.7, pNMG-624, ABE3.2, ABE5.3, pNMG-558, pNMG-576, pNMG-577, pNMG-586, ABE7.2, pNMG-620, pNMG-617, pNMG-618, pNMG-620, pNMG-621, pNGM-622, pNMG-623, ABE6.3, ABE6.4, ABE7.8, ABE7.9, ABE7.10, ABEMax, ABE8e, CP1028-ABE8e, ABE7.10-CP1041, CP1041-ABE8e, or any combination thereof. In some embodiments, the base editor comprises an adenine base editor (ABE) and/or a cytosine base editor (CBE). In some embodiments, the ABE comprises monomer and dimer versions of one or more of ABE8e, ABE8e-V106W, SaABE8e, SaKKH-ABE8e, NG-ABE8e, ABE-xCas9, ABE8e-NRTH, ABE8e-NRRH, ABE8e-NRCH, ABE8e-NG-CP1041, ABE8e-VRQR-CP1041, ABE8e-CP1041, ABE8e-CP1028, ABE8e-VRQR, ABE8e-LbCas12a (LbABE8e), ABE8e-AsCas12a (enAsABE8e), ABE8e-SpyMac, ABE8e (TadA-8e V106W), ABE8e (K20A,R21A), and ABE8e (TadA-8e V82G). In some embodiments, p is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. In some embodiments, n is 2, 3, 4, 5, 6, 7, 8, 9, 10, 11, 12, 13, 14, or 15. In some embodiments, n=m. In some embodiments, m is 2, 3, 4, 5, 6, 7, 8, 9, 10, 11, 12, 13, 14, or 15. In some embodiments, one or more polynucleotides encoding components of the system are capable of being introduced into a cell in a single step (e.g., via piggyBac transposition). In some embodiments, the length of the hypercascade array is at least, or most, about 200 bp, 400 bp, 600 bp, 800 bp, 1.0 kb, 1.5 kb, 2.0 kb, 2.5 kb, 3.0 kb, 3.5 kb, 4.0 kb, 4.5 kb, 5.0 kb, 5.5 kb, 6.0 kb, 6.5 kb, 7.0 kb, or 7.5 kb. In some embodiments, the system capable of linear editing for an increased period before saturation as compared to non-regenerative target arrays. In some embodiments, the hypercascade array comprises an internal priming site capable of binding a custom sequencing primer, thereby enabling recovery of the entire hypercascade array along with the first and/or second static barcode(s) via a short-read sequencing protocol (e.g., via 3′ gene expression protocol).

Disclosed herein include nucleic acid compositions. In some embodiments, the nucleic acid composition comprises: one or more first polynucleotide(s) encoding one or more hypercascade arrays disclosed herein; one or more second polynucleotide(s) encoding n layer guide RNAs (gRNAs) disclosed herein; and/or one or more third polynucleotide(s) encoding an editor disclosed herein. In some embodiments, one or more of the first polynucleotide(s), the second polynucleotide(s), and the third polynucleotide(s) are operably connected to a promoter selected from the group comprising: an RNA pol I promoter; a pol II promoter (e.g., CMV, SV40 early region or adenovirus major late promoter); a pol III promoter (e.g., a U6 or H1 promoter); a minimal promoter (e.g., TATA, miniCMV, and/or miniPromo); a bacteriophage promoter (e.g., a bacteriophage T3 promoter, a bacteriophage T7 promoter, a bacteriophage SP6 promoter, or a combination thereof); a tissue-specific promoter; a lineage-specific promoter; and/or a ubiquitous promoter (e.g., a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CASI promoter, a CBH promoter, or any combination thereof).

In some embodiments, the nucleic acid composition is complexed or associated with one or more lipids or lipid-based carriers, thereby forming liposomes, lipid nanoparticles (LNPs), lipoplexes, and/or nanoliposomes (e.g., encapsulating the nucleic acid composition). In some embodiments, the nucleic acid composition is, comprises, or further comprises, one or more vectors. In some embodiments, at least one of the one or more vectors is a viral vector, a plasmid, a transposable element, a naked DNA vector, a lipid nanoparticle (LNP), or any combination thereof. In some embodiments, the viral vector is an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof. In some embodiments, the transposable element is piggybac transposon or sleeping beauty transposon. In some embodiments, the target array(s) are flanked with piggyBac inverted terminal repeats. In some embodiments, a T7 promoter is situated upstream of the target array(s). In some embodiments, the first polynucleotide(s), the second polynucleotide(s), and/or the third polynucleotide(s) are comprised in the one or more vectors. In some embodiments, the first polynucleotide(s), the second polynucleotide(s), and/or the third polynucleotide(s) are comprised in the same vector and/or different vectors. In some embodiments, the first polynucleotide(s), the second polynucleotide(s), and/or the third polynucleotide(s) are situated on the same nucleic acid and/or different nucleic acids. In some embodiments, the hypercascade array(s) are flanked with piggyBac inverted terminal repeats.

Disclosed herein include populations of cells. In some embodiments, the population of cells comprises: one or more hypercascade array(s) disclosed herein; n layer guide RNAs (gRNAs) disclosed herein; and/or an editor disclosed herein. In some embodiments, the population of cells comprises: one or more first polynucleotides encoding one or more hypercascade array(s) disclosed herein; one or more second polynucleotides encoding n layer guide RNAs (gRNAs) disclosed herein; and/or one or more third polynucleotides encoding an editor disclosed herein. In some embodiments, the population of cells comprises at least about 10, 100, 1000, 10000, 25000, 50000, 75000, 100000, 500000, or 1000000 cells. In some embodiments, the population of cells comprises one or more of the following: a primary cell, an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. In some embodiments, the stem cell comprises an embryonic stem cell, an induced pluripotent stem cell (iPSC), a hematopoietic stem/progenitor cell (HSPC), or any combination thereof.

Disclosed herein include methods. In some embodiments, the method comprises: introducing the one or more hypercascade array(s) disclosed herein, the n layer guide RNAs (gRNAs) disclosed herein, and the editor disclosed herein into a cell or a first population of cells; incubating the cell(s) for a period of time; and/or obtaining sequence information of the hypercascade array(s) of each of the resulting second population of cells (e.g., via sequencing or imaging). In some embodiments, the introducing step comprises introducing a nucleic acid composition disclosed herein into the cell(s). In some embodiments, the cell(s) undergo at least about 2, 3, 4, 5, 6, 7, 8, 9, 10, 11, 12, 13, 14, 15, 16, 17, 18, 19, or 20, generations during the incubation. In some embodiments, the period of time comprises at least about 6 hr, 12 hr, 18 hr, 24 hr, 1 day, 2 days, 3 days, 4 days, 5 days, 6 days, 7 days, 10 days, 2 weeks, 3 weeks, or 4 weeks. In some embodiments, the method further comprises generating a transcriptomic profile of each of the resulting second population of cells. In some embodiments, the method comprises reconstructing lineage relationships between the resulting second population of cells. In some embodiments, edits accumulate linearly over a period of time of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days. In some embodiments, the first polynucleotide(s), the second polynucleotide(s), and/or the third polynucleotide(s) become integrated in the genome of the population of cell(s) after the introducing step. In some embodiments, two or more hypercascade arrays are integrated into a genome of at least one cell, and wherein the different integrations are capable of being distinguished from one another via the first static barcode and/or second static barcode(s).

In some embodiments, the method comprises exposing the cell(s) to one or more agents. In some embodiments, the one or more agents comprise: one or more of a chemical agent, a pharmaceutical, small molecule, a biologic, a CRISPR single-guide RNA (sgRNA), a small interfering RNA (siRNA), CRISPR RNA (crRNA), a small hairpin RNA (shRNA), a microRNA (miRNA), a piwi-interacting RNA (piRNA), an antisense oligonucleotide, a peptide or peptidomimetic inhibitor, an aptamer, an antibody, an intrabody, or any combination thereof; an expression vector, wherein the expression vector encodes one or more of the following: an mRNA, an antisense nucleic acid molecule, a RNAi molecule, a shRNA, a mature miRNA, a pre-miRNA, a pri-miRNA, an anti-miRNA, a ribozyme, any combination thereof; an infectious agent, an anti-infectious agent, or a mixture thereof; a cytotoxic agent, optionally a chemotherapeutic agent, a biologic agent, a toxin, a radioactive isotope, or any combination thereof; one or more of an epigenetic modifying agent, epigenetic enzyme, a bicyclic peptide, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis inhibitor, a nuclease, a protein fragment or domain, a tag or marker, an antigen, an antibody or antibody fragment, a ligand or a receptor, a synthetic or analog peptide from a naturally-bioactive peptide, an anti-microbial peptide, a pore-forming peptide, a targeting or cytotoxic peptide, a degradation or self-destruction peptide, a CRISPR component system or component thereof, DNA, RNA, artificial nucleic acids, a nanoparticle, an oligonucleotide aptamer, a peptide aptamer, or any combination thereof; and/or at least one effector activity selected from the group consisting of: modulating a biological activity, binding a regulatory protein, modulating enzymatic activity, modulating substrate binding, modulating receptor activation, modulating protein stability/degradation, modulating transcript stability/degradation, or any combination thereof.

Disclosed herein include systems. In some embodiments, the system comprises: (i) one or more target array(s) comprising n editable target sites, wherein n is an integer greater than 1; (ii) n guide RNAs (gRNAs); and/or (iii) a base editor capable of adenine (A)-to-guanine (G) base editing. In some embodiments, each guide RNA of the n gRNAs comprises a unique spacer sequence capable of binding one of the n editable target sites. In some embodiments, each editable target site of the n editable target sites comprises an AA dinucleotide and a unique protospacer sequence relative to the other n editable target sites. In some embodiments, the base editor associated with one of the n gRNAs is capable of editing the AA dinucleotide to a GA dinucleotide, AG dinucleotide, or GG dinucleotide. In some embodiments, a GA dinucleotide and an AG dinucleotide each disrupt binding to the gRNA, and thereby edited target sites comprising a GA dinucleotide and an AG dinucleotide are incapable of being further edited to a GG dinucleotide.

In some embodiments, the base editor comprises an adenine base editor (ABE). In some embodiments, the ABE comprises monomer and dimer versions of one or more of ABE8e, ABE8e-V106W, SaABE8e, SaKKH-ABE8e, NG-ABE8e, ABE-xCas9, ABE8e-NRTH, ABE8e-NRRH, ABE8e-NRCH, ABE8e-NG-CP1041, ABE8e-VRQR-CP1041, ABE8e-CP1041, ABE8e-CP1028, ABE8e-VRQR, ABE8e-LbCas12a (LbABE8e), ABE8e-AsCas12a (enAsABE8e), ABE8e-SpyMac, ABE8e (TadA-8e V106W), ABE8e (K20A,R21A), ABE8e (TadA-8e V82G), ABE7.7, pNMG-624, ABE3.2, ABE5.3, ABE7.2, pNMG-620, pNMG-617, pNMG-618, pNMG-620, pNMG-621, pNGM-622, pNMG-623, ABE6.3, ABE6.4, ABE7.8, ABE7.9, ABE7.10, ABEMax, ABE8e, CP1028-ABE8e, ABE7.10-CP1041, CP1041-ABE8e, or any combination thereof.

In some embodiments, n is 2, 3, 4, 5, 6, 7, 8, 9, 10, 11, 12, 13, 14, 15, 16, 17, 18, 19, or 20. In some embodiments, the n editable target sites are in tandem. In some embodiments, the editable target site is about 20 bp to about 40 bp in length. In some embodiments, one or more target array(s) comprise at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 75, 80, 85, 90, 95, 100, 105, 110, 115, 120, 125, 130, 135, 140, 145, 150, 155, 160, 165, 170, 175, 180, 185, 190, 195, 200 target arrays. In some embodiments, each target array comprises: a first static barcode; and/or one or more second static barcodes (e.g., two second static barcodes). In some embodiments, the first static barcode is about 10 nt in length. In some embodiments, the first static barcode is selected from a library of at least about 106 different first static barcode sequences. In some embodiments, the one or more second static barcodes are image-readable. In some embodiments, a second static barcode is selected from a library of at least about 200 different second static barcode sequences.

In some embodiments, the gRNA is a single guide RNA (sgRNA). In some embodiments, the expression of the n guide RNAs are under the control of a first inducible promoter. In some embodiments, the first inducible promoter is capable of inducing transcription in the presence of a first agent. In some embodiments, the first inducible promoter comprises a Wnt-responsive element (WRE). In some embodiments, the first agent is a Wnt signaling ligand (e.g., GSK-3 inhibitor CHIR99021 (CHIR)). In some embodiments, the expression of the base editor is under the control of a second inducible promoter. In some embodiments, the second inducible promoter is capable of inducing transcription in the presence of a second agent. In some embodiments, the second agent comprises tetracycline, doxycycline or a derivative thereof. In some embodiments, the inducible second promoter comprises one or more copies of a transactivator recognition sequence a transactivator is capable of binding to induce transcription. In some embodiments, the transactivator is incapable of binding the transactivator recognition sequence in the absence of a transactivator-binding compound. In some embodiments, the one or more copies of a transactivator recognition sequence comprise one or more copies of a tet operator (TetO). In some embodiments, the second agent comprises the transactivator-binding compound. In some embodiments, one or more polynucleotides encoding components of the system are capable of being introduced into a cell in a single step (e.g., via piggyBac transposition).

Disclosed herein include nucleic acid compositions. In some embodiments, the nucleic acid composition comprises: one or more first polynucleotide(s) encoding the one or more target array(s) disclosed herein; one or more second polynucleotide(s) encoding the n guide RNAs (gRNAs) disclosed herein; and/or one or more third polynucleotide(s) encoding the base editor disclosed herein.

In some embodiments, the one or more second polynucleotide(s) are operably connected to a first inducible promoter and the one or more third polynucleotide(s) are operably connected to a second inducible promoter. In some embodiments, one or more of the first polynucleotide(s), the second polynucleotide(s), and the third polynucleotide(s) are operably connected to a promoter selected from the group comprising: an RNA pol I promoter; a pol II promoter (e.g., CMV, SV40 early region or adenovirus major late promoter); a pol III promoter (e.g., a U6 or H1 promoter); a minimal promoter (e.g., TATA, miniCMV, and/or miniPromo); a bacteriophage promoter (e.g., a bacteriophage T3 promoter, a bacteriophage T7 promoter, a bacteriophage SP6 promoter, or a combination thereof); a tissue-specific promoter; a lineage-specific promoter; and/or a ubiquitous promoter (e.g., a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CASI promoter, a CBH promoter, or any combination thereof).

In some embodiments, the nucleic acid composition is complexed or associated with one or more lipids or lipid-based carriers, thereby forming liposomes, lipid nanoparticles (LNPs), lipoplexes, and/or nanoliposomes (e.g., encapsulating the nucleic acid composition). In some embodiments, the nucleic acid composition is, comprises, or further comprises, one or more vectors. In some embodiments, at least one of the one or more vectors is a viral vector, a plasmid, a transposable element, a naked DNA vector, a lipid nanoparticle (LNP), or any combination thereof. In some embodiments, the viral vector is an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof. In some embodiments, the transposable element is piggybac transposon or sleeping beauty transposon. In some embodiments, the target array(s) are flanked with piggyBac inverted terminal repeats. In some embodiments, a T7 promoter is situated upstream of the target array(s). In some embodiments, the first polynucleotide(s), the second polynucleotide(s), and/or the third polynucleotide(s) are comprised in the one or more vectors. In some embodiments, the first polynucleotide(s), the second polynucleotide(s), and/or the third polynucleotide(s) are comprised in the same vector and/or different vectors. In some embodiments, the first polynucleotide(s), the second polynucleotide(s), and/or the third polynucleotide(s) are situated on the same nucleic acid and/or different nucleic acids.

Disclosed herein include populations of cells. In some embodiments, the population of cells comprises: one or more target array(s) disclosed herein; n guide RNAs (gRNAs) disclosed herein; and/or a base editor disclosed herein. In some embodiments, the population of cells comprises: one or more first polynucleotide(s) encoding one or more target array(s) disclosed herein; one or more second polynucleotide(s) encoding n guide RNAs (gRNAs) disclosed herein; and/or one or more third polynucleotide(s) encoding a base editor disclosed herein.

In some embodiments, the cells comprise integrated reverse tetracycline-controlled transactivator (rtTA). In some embodiments, the population of cells comprises at least about 10, 100, 1000, 10000, 25000, 50000, 75000, 100000, 500000, or 1000000 cells. In some embodiments, the population of cells comprises one or more of the following: a primary cell, an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. In some embodiments, the stem cell comprises an embryonic stem cell, an induced pluripotent stem cell (iPSC), a hematopoietic stem/progenitor cell (HSPC), or any combination thereof.

Disclosed herein include methods. In some embodiments, the method comprises: introducing one or more target array(s) disclosed herein, n guide RNAs (gRNAs) disclosed herein, and a base editor disclosed herein into a cell or a first population of cells; incubating the cell(s) for a period of time; and/or obtaining sequence information of the one or more target array(s) of each of the resulting second population of cells (e.g., via sequencing or imaging). In some embodiments, the introducing step comprises introducing a nucleic acid composition disclosed herein into the cell(s).

In some embodiments, the method comprises contacting the cells with the first agent and/or the second agent to induce expression of the n guide RNAs and/or the base editor, respectively. In some embodiments, imaging comprises single molecule RNA FISH (smFISH). In some embodiments, the method comprises fixing the resulting second population of cells and in situ T7 transcription. In some embodiments, the cell(s) undergoes at least about 2, 3, 4, 5, 6, 7, 8, 9, 10, 11, 12, 13, 14, 15, 16, 17, 18, 19, or 20, generations during the incubation. In some embodiments, the period of time comprises at least about 6 hr, 12 hr, 18 hr, 24 hr, 1 day, 2 days, 3 days, 4 days, 5 days, 6 days, 7 days, 10 days, 2 weeks, 3 weeks, or 4 weeks. In some embodiments, the method further comprises generating a transcriptomic profile of each of the resulting second population of cells.

In some embodiments, the method comprises reconstructing lineage relationships between the resulting second population of cells. In some embodiments, lineage can be accurately reconstructed for at least about 4, 5, 6, 7, 8, 9, 10, 11, or 12 generations. In some embodiments, the method comprises deriving correlations between lineage history, spatial position, and cell fate. In some embodiments, the method comprises combined the lineage reconstruction with spatial and cell state dynamics to infer the relative spatial mobilities of different cell states. In some embodiments, edits accumulate over a period of time of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days. In some embodiments, the first polynucleotide(s), the second polynucleotide(s), and/or the third polynucleotide(s) become integrated in the genome of the cell(s) after the introducing step. In some embodiments, the two or more target array(s) are integrated into a genome of at least one cell, and wherein the different integrations are capable of being distinguished from one another via the first static barcode and/or second static barcode(s).

In some embodiments, the method comprises exposing the cell(s) to one or more agents. In some embodiments, the one or more agents comprise: one or more of a chemical agent, a pharmaceutical, small molecule, a biologic, a CRISPR single-guide RNA (sgRNA), a small interfering RNA (siRNA), CRISPR RNA (crRNA), a small hairpin RNA (shRNA), a microRNA (miRNA), a piwi-interacting RNA (piRNA), an antisense oligonucleotide, a peptide or peptidomimetic inhibitor, an aptamer, an antibody, an intrabody, or any combination thereof; an expression vector, wherein the expression vector encodes one or more of the following: an mRNA, an antisense nucleic acid molecule, a RNAi molecule, a shRNA, a mature miRNA, a pre-miRNA, a pri-miRNA, an anti-miRNA, a ribozyme, any combination thereof; an infectious agent, an anti-infectious agent, or a mixture thereof; a cytotoxic agent, optionally a chemotherapeutic agent, a biologic agent, a toxin, a radioactive isotope, or any combination thereof; one or more of an epigenetic modifying agent, epigenetic enzyme, a bicyclic peptide, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis inhibitor, a nuclease, a protein fragment or domain, a tag or marker, an antigen, an antibody or antibody fragment, a ligand or a receptor, a synthetic or analog peptide from a naturally-bioactive peptide, an anti-microbial peptide, a pore-forming peptide, a targeting or cytotoxic peptide, a degradation or self-destruction peptide, a CRISPR component system or component thereof, DNA, RNA, artificial nucleic acids, a nanoparticle, an oligonucleotide aptamer, a peptide aptamer, or any combination thereof; and/or at least one effector activity selected from the group consisting of: modulating a biological activity, binding a regulatory protein, modulating enzymatic activity, modulating substrate binding, modulating receptor activation, modulating protein stability/degradation, modulating transcript stability/degradation, or any combination thereof.

### BRIEF DESCRIPTION OF THE DRAWINGS

FIG. 1A-FIG. 1E depict non-limiting exemplary embodiments and data related to multiplexed, genomically dispersed, editable barcodes that enable detailed recording of lineages over many generations with in situ readout. FIG. 1A depicts that detailed lineage trees can be measured alongside transcriptional cell states while maintaining spatial context through phylogenetic barcoding. FIG. 1B depicts that predicted stochastic editing of AA dinucleotides results in one of three terminal outcomes. FIG. 1C depicts that an inducible barcode editing system can be integrated into cells at high copy number via piggyBac transposase. Target arrays (top panel) contained 6 AA dinucleotides flanked by unique protospacer sequences as well as sequencing and imaging-readable static barcodes which served to uniquely mark different genomic integrations of the array. Editing was induced by expression of guide RNAs (middle panel), controlled by a Wnt-responsive element, and base editor (bottom panel), controlled by the TRE3G tet-on promoter. FIG. 1D depicts an engineered a monoclonal mESC cell line containing 66 uniquely labeled target array copies (e.g., 396 editable dinucleotides, or 792 bits of information) alongside the inducible editing machinery. FIG. 1E depicts that the engineered cell line in FIG. 1D enabled genomic lineage recording and recovery through FISH imaging and phylogenetic tree inference.

FIG. 2A-FIG. 2C depict non-limiting exemplary embodiments and data related to dinucleotide targets accumulating edits over time in engineered mESCs. FIG. 2A depicts that next generation amplicon sequencing quantified editing over time after induction of gRNAs and ABE. FIG. 2B depicts all targets edited over time in the presence of the two inducers together, although at distinct rates (Dox+CHIR). As shown in FIG. 2B, Dox induction alone drove editing at a slower rate. In the absence of dox, editing did not proceed at an appreciable rate (CHIR and control). Three biological replicates were collected for each time point in FIG. 2B. The solid line shows the fit for a probabilistic model of editing over time. FIG. 2C depicts that each target had a unique distribution of editing outcomes that remained constant as editing progresses.

FIG. 3A-FIG. 3F depict non-limiting exemplary embodiments and data showing that multiple rounds of Zombie-FISH recovered dynamic and static barcode states. FIG. 3A depicts that barcode states can be recovered across multiple rounds of microscopic imaging. Ectopic application of T7 polymerase generated localized RNA clusters. Primary DNA probes were bound to the dynamic and static barcodes as well as to endogenous transcripts, competing primary probes against each other for binding to the different possible dynamic barcode variants. Each primary probe had an overhang sequence allowing for binding of one or more fluorescently labeled secondary probes, which were hybridized, imaged, and stripped away sequentially to recover barcoding information as shown in FIG. 3B and transcriptional information as shown in FIG. 3C. In FIG. 3B and FIG. 3C, scale bars are 20 μm. FIG. 3D depicts that across 8 colonies, 50-80% of target arrays were recovered per cell. One colony (e.g., colony 1) had dramatically lower barcode recovery and was excluded from further analysis. FIG. 3E depicts that each unique target array was recovered in a similar fraction of cells. FIG. 3F depicts that approximately 200 dinucleotide dynamic targets were recovered with high confidence per cell, with around 100 of these measured jointly between any pair of cells

FIG. 4A-FIG. 4C depict non-limiting exemplary embodiments and data showing that lineage can be accurately reconstructed for at least 12 generations in simulation. FIG. 4A depicts that to estimate the expected accuracy of reconstruction, cell division and stochastic editing were simulated, starting with unedited barcodes, represented as sets of AA dinucleotides (left panel labeled as endpoint cells) over time to produce heterogeneous edit patterns. Then, either all sequences were retained or 50% of the data was dropped to represent random FISH detection losses. Cells that had few barcode characters overlapping with those measured in other cells (right) were filtered out. FIG. 4B depicts that based on these ground truth simulations, lineage relationships were reconstructed. The Robinson-Foulds distance between the ground truth input tree and reconstructed output tree (e.g., posterior tree distribution) was also computed. FIG. 4C depicts that reconstruction accuracy was nearly perfect without barcode dropout (dark blue dots). With dropout, about 10% error rates were observed with tree depths up to 12 cell generations (gray dots). In the presence of dropout, filtering cells with few shared units moderately improved the reconstructed tree (light blue dots).

FIG. 5A-FIG. 5H depict non-limiting exemplary embodiments and data showing that joint measurements of lineage, gene expression and spatial position revealed cell state transition dynamics. FIG. 5A depicts lineage relationships recorded in mESC cells cultured in serum-LIF media over a 3-day period. Editing was induced with 3 μM CHIR (FIG. 5B) and 1 μM Dox (FIG. 5C). Cells were clustered into 5 states based on gene expression as measured by smFISH. As shown in FIG. 5C, two clusters were well separated from the other groups, while three clusters appeared continuously related and expressed different levels of key marker genes (see also FIG. 9A). FIG. 5D-FIG. 5G depict topological lineage tree relationships, cell division timing, ancestral cell states, and transition rates between those states inferred from lineage reconstruction. Uncertainty in lineage tree measurements was visualized by overlaying trees sampled from the posterior distribution of trees generated by Markov chain Monte Carlo for each colony, with results shown in top panel of FIG. 5D (see also FIG. 10). Cell states and clade groups from the lineage tree were mapped to the spatial colony images to qualitatively inspect the relationships between cell state, lineage, and spatial, with results shown in bottom panel of FIG. 5D (see also FIG. 10). FIG. 5E depicts that spatial distance was larger between cells with more distant common ancestors. FIG. 5F depicts that several cell state transitions were inferred to have non-zero median values across all posterior samples. FIG. 5G depicts that these state transitions predicted a restricted cell state transition graph. One transition (denoted by *) contained a high fraction of posterior samples with a transition rate of 0. Numbers in FIG. 5G indicate the median expected number of transitions per day for cells of the given type. FIG. 5H depicts that several doublet motifs were significantly over- or under-represented across the lineage tree posterior samples. Abbreviations in FIG. 5H are: N (Naive), 2 (2C-like), F (Formative), N2 (Naive, trending to 2C-like), NF (Naive, trending to formative) and MRCA (Most recent common ancestor).

FIG. 6A-FIG. 6B depict non-limiting exemplary embodiments and data related to 66 unique integrations detected in the baseMEM-01 cell line. 66 barcode integrations were identified by next generation sequencing of target arrays amplified from genomic DNA. The number of reads corresponding to unique sequenceable barcodes (FIG. 6A) and image readable static barcodes (FIG. 6B) was quantified, identifying approximately 66 variants in each case. The top 200 most frequent variants are shown in FIG. 6A and FIG. 6B. True variants were separated from noise heuristically by identifying the “knee of the curve” (dashed vertical lines). Importantly, these 66 variants were all also identified in the FISH experiments shown in FIG. 3E.

FIG. 7A-FIG. 7E depict non-limiting exemplary embodiments and data related to a support vector machine that classified barcode states based on fluorescence measurements. FIG. 7A depicts that manually annotated barcodes were correctly classified by a quadratic kernel support vector machine (SVM) approximately 94% of the time. FIG. 7B depicts that classification probability estimates were very high within the training dataset (left panel of FIG. 7B). Outside of the training sample, most classification probabilities were still high but with a subset of predictions that were less certain (right panel of FIG. 7B). The support vector machine predicted classes based on 16 fluorescence measurements corresponding to each pseudo-color as defined in FIG. 3B. FIG. 7C depicts that each class was well separated based on these features. FIG. 7D depicts that after 3 days of editing induction, many dynamic barcodes were identified as class 2, corresponding to the unedited state (left panel of FIG. 7D). Static barcode classifications were more evenly distributed, as anticipated (right panel of FIG. 7D). FIG. 7E depicts that static barcodes decoded by FISH typically perfectly matched the 66 image readable barcode sequences identified by sequencing as shown in FIG. 6B, although a fraction of barcodes were recovered with one or more character differences relative to their closest match.

FIG. 8A-FIG. 8B depict non-limiting exemplary embodiments and data showing that stochastic simulations closely recapitulated the empirical editing process. FIG. 8A depicts that a stochastic editing simulator was developed based on the Gillespie algorithm that closely recapitulated the average edit accumulation model developed and illustrated in FIG. 2B. FIG. 8B depicts that the simulated edit outcome distributions for each target site matched the observed distributions shown in FIG. 2C.

FIG. 9A-FIG. 9B depict non-limiting exemplary embodiments and data related to mESC gene expression clustering. FIG. 9A depicts that principal component analysis was largely in agreement with nonlinear dimensionality reduction, with separation between major clusters observed along the first three components. The naive states also appeared continuously related in this view (FIG. 9A). FIG. 9B depicts that clusters have distinct marker gene expression patterns, with some similarity between the Naive/2C-like, Naive and Naive/Formative states.

FIG. 10 depicts non-limiting exemplary embodiments and data related to lineage relationships, cell states, and spatial positions across multiple colonies revealed by baseMEMOIR. Posterior tree distributions were visualized and mapped back to illustrations of each colony as in FIG. 5D.

FIG. 11 depicts non-limiting exemplary embodiments and data related to gene detection that was consistent across images. Gene counts as quantified from FISH images by the bigFISH package were correlated for cells that were measured in multiple images.

FIG. 12A-FIG. 12F depict non-limiting exemplary embodiments and data showing that the hypercascade system linearized edit rate over time and densely packed mutable target sites. FIG. 12A depicts that the CRISPR A-to-G base editor makes predictable mutations at defined target sites. FIG. 12B depicts that reading out heritable mutations enabled reconstruction of cellular lineage relationships. FIG. 12C depicts that arrays of independently edited targets were exponentially lost over time, while a system that generated new targets over the course of editing would maintain constant edit rate over an extended time scale. FIG. 12D depicts that new targets can be generated over time by repairing protospacer and PAM mismatches through A-to-G mutations. FIG. 12E depicts that the concept disclosed herein can be extended to multiple unlocking layers of densely packed target sites. This sequence comprised a tandem repeating 20-mer which was acted on by four unique guide RNAs. Mismatches that were repaired to generate new targets are indicated with arrows. FIG. 12F depicts that simulations of editing in this scheme revealed linear edit accumulation relative to arrays of independent targets.

FIG. 13A-FIG. 13C depict non-limiting exemplary embodiments and data related to hypercascades that allowed lineage recording and reconstruction across a broader range of target copy numbers and edit rates compared to arrays of independent sites. FIG. 13A depicts that stochastic simulations of target editing and cell division together enabled comparison of lineage reconstruction accuracy between independent and hypercascading systems. FIG. 13B depicts that hypercascade broadened the range of acceptable copy numbers and edit rates for reconstruction compared to an independent array with the same sequence length. By holding tree depth constant, the hypercascade system outperformed an independent array of targets with comparable sequence length over a variety of edit rates and copy numbers. FIG. 13C depicts that hypercascade outperformed independent arrays with the same number of target sites in some regimes. The difference was more nuanced comparing the hypercascade to independent arrays with the same total number of targets.

FIG. 14A-FIG. 14D depict non-limiting exemplary embodiments and data related to the hypercascade exhibiting sequential editing in living cells. FIG. 14A depicts that three different implementations of the hypercascade were stably integrated into a mouse embryonic kidney line with either on- or off-target gRNAs. FIG. 14B depicts that editing occurred only in the presence of on-target gRNAs. FIG. 14C depicts that edits accumulated linearly over a 44 day period. FIG. 14D depicts that layers of targets were sequentially activated and well fit by a stochastic model of editing.

FIG. 15A-FIG. 15G depict non-limiting exemplary embodiments and data showing the hypercascade can be integrated in a single step and used for subsequent lineage recording. FIG. 15A depicts that all components of the system were simultaneously integrated into hiPSCs. FIG. 15B depicts edits accrued on integrated targets over a 23-day time course, which was observed after a delay period. FIG. 15C depicts that layered targets were sequentially activated. FIG. 15D depicts that read counts binned by total edits presented a multimodal distribution with peaks that shifted right over time. FIG. 15E depicts that hierarchical clustering reconstructed lineage relationships among 3289 unique barcode sequences observed with at least 22 accumulated edits. FIG. 15F depicts that the average tip depth was estimated to be approximately 17 generations. FIG. 15G depicts that the mean cophenetic distance between pairs of cells from distinct edit rate groups was lower than in a randomized barcode control, identifying edit rate as a clonal feature.

FIG. 16A-FIG. 16E depict non-limiting exemplary embodiments and data related to the BigMEMOIR (e.g., baseMEMOIR) line containing editable barcodes. FIG. 16A depicts that the editable barcodes allowed for sequential FISH readout analysis. FIG. 16B depicts that fluorescent images of barcodes collected across multiple rounds of hybridization obtained using FISH techniques were categorized by machine learning to identify the brightest fluorescence against four potential pseudo-colors, decoding dynamic and static barcode sequences. FIG. 16C depicts that cell states were clustered by expression of pluripotent stem cell state markers. Cell state data was paired with lineage data to correlate cell state relationships with lineage and physical location. FIG. 16D depicts that long barcodes with 396 characters were determined for each cell, combining information from all target array copies. Dynamic barcodes allowed for reconstruction of phylogenetic lineage relationships. In FIG. 16E, the colony is colored based on cell state (pink and orange) as well as relative placement on the phylogenetic tree (red to blue), with warm colors and cool colors representing earlier splits into clades. More subtle shades within a hue represent more closely related cells.

FIG. 17 depicts a non-limiting exemplary schematic illustrating how stochastic edits in the hypercascade array activate new layers over time. In some embodiments, for deeper layered gRNA sites to be activated, edits must have already been made in both adjacent target sites at the upper level.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include systems. In some embodiments, the system comprises: (i) one or more hypercascade array(s) each comprising p hypercascade units, wherein p is an integer greater than 1; (ii) n layer guide RNAs (gRNAs), wherein n is an integer greater than 1; and/or (iii) an editor. In some embodiments, the editor is a base editor capable of base editing the hypercascade array. In some embodiments, said base editing comprises: adenine (A)-to-guanine (G) base editing and/or cytosine (C)-to-thymine (T) base editing. In some embodiments, each hypercascade unit comprises m target sites, and wherein m is an integer greater than 1. In some embodiments, the target sites comprises one or more primary target site(s) and one or more conditional target site(s). In some embodiments, the target sites each comprise an editable base. In some embodiments, the editor associated with a layer gRNA is capable of editing said editable base of a target site comprising a complementary protospacer and a protospacer adjacent motif (PAM). In some embodiments, the primary target sites are capable of being edited by a first layer gRNA and the editor. In some embodiments, each of the one or more conditional target sites comprise two or more mismatches in the protospacer and/or the PAM. In some embodiments, the editing of adjacent editable bases by a previous layer gRNA are capable of repairing said mismatches, thereby enabling the editing of a conditional target site by a layer gRNA and the editor. Disclosed herein include nucleic acid compositions. In some embodiments, the nucleic acid composition comprises: one or more first polynucleotide(s) encoding one or more hypercascade arrays disclosed herein; one or more second polynucleotide(s) encoding n layer guide RNAs (gRNAs) disclosed herein; and/or one or more third polynucleotide(s) encoding an editor disclosed herein. Disclosed herein include populations of cells. In some embodiments, the population of cells comprises: one or more hypercascade array(s) disclosed herein; n layer guide RNAs (gRNAs) disclosed herein; and/or an editor disclosed herein. In some embodiments, the population of cells comprises: one or more first polynucleotides encoding one or more hypercascade array(s) disclosed herein; one or more second polynucleotides encoding n layer guide RNAs (gRNAs) disclosed herein; and/or one or more third polynucleotides encoding an editor disclosed herein. Disclosed herein include methods. In some embodiments, the method comprises: introducing the one or more hypercascade array(s) disclosed herein, the n layer guide RNAs (gRNAs) disclosed herein, and the editor disclosed herein into a cell or a first population of cells; incubating the cell(s) for a period of time; and/or obtaining sequence information of the hypercascade array(s) of each of the resulting second population of cells (e.g., via sequencing or imaging). In some embodiments, the introducing step comprises introducing a nucleic acid composition disclosed herein into the cell(s).

Disclosed herein include systems. In some embodiments, the system comprises: (i) one or more target array(s) comprising n editable target sites, wherein n is an integer greater than 1; (ii) n guide RNAs (gRNAs); and/or (iii) a base editor capable of adenine (A)-to-guanine (G) base editing. In some embodiments, each guide RNA of the n gRNAs comprises a unique spacer sequence capable of binding one of the n editable target sites. In some embodiments, each editable target site of the n editable target sites comprises an AA dinucleotide and a unique protospacer sequence relative to the other n editable target sites. In some embodiments, the base editor associated with one of the n gRNAs is capable of editing the AA dinucleotide to a GA dinucleotide, AG dinucleotide, or GG dinucleotide. In some embodiments, a GA dinucleotide and an AG dinucleotide each disrupt binding to the gRNA, and thereby edited target sites comprising a GA dinucleotide and an AG dinucleotide are incapable of being further edited to a GG dinucleotide. Disclosed herein include nucleic acid compositions. In some embodiments, the nucleic acid composition comprises: one or more first polynucleotide(s) encoding the one or more target array(s) disclosed herein; one or more second polynucleotide(s) encoding the n guide RNAs (gRNAs) disclosed herein; and/or one or more third polynucleotide(s) encoding the base editor disclosed herein. Disclosed herein include populations of cells. In some embodiments, the population of cells comprises: one or more target array(s) disclosed herein; n guide RNAs (gRNAs) disclosed herein; and/or a base editor disclosed herein. In some embodiments, the population of cells comprises: one or more first polynucleotide(s) encoding one or more target array(s) disclosed herein; one or more second polynucleotide(s) encoding n guide RNAs (gRNAs) disclosed herein; and/or one or more third polynucleotide(s) encoding a base editor disclosed herein. Disclosed herein include methods. In some embodiments, the method comprises: introducing one or more target array(s) disclosed herein, n guide RNAs (gRNAs) disclosed herein, and a base editor disclosed herein into a cell or a first population of cells; incubating the cell(s) for a period of time; and/or obtaining sequence information of the one or more target array(s) of each of the resulting second population of cells (e.g., via sequencing or imaging). In some embodiments, the introducing step comprises introducing a nucleic acid composition disclosed herein into the cell(s).

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the terms “nucleic acid” and “polynucleotide” are interchangeable and refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

The term “vector” as used herein, can refer to a vehicle for carrying or transferring a nucleic acid. Non-limiting examples of vectors include plasmids and viruses (for example, AAV viruses).

The term “construct,” as used herein, refers to a recombinant nucleic acid that has been generated for the purpose of the expression of a specific nucleotide sequence(s), or that is to be used in the construction of other recombinant nucleotide sequences.

As used herein, the term “plasmid” refers to a nucleic acid that can be used to replicate recombinant DNA sequences within a host organism. The sequence can be a double stranded DNA.

The term “element” refers to a separate or distinct part of something, for example, a nucleic acid sequence with a separate function within a longer nucleic acid sequence. The term “regulatory element” and “expression control element” are used interchangeably herein and refer to nucleic acid molecules that can influence the expression of an operably linked coding sequence in a particular host organism. These terms are used broadly to and cover all elements that promote or regulate transcription, including promoters, core elements required for basic interaction of RNA polymerase and transcription factors, upstream elements, enhancers, and response elements (see, e.g., Lewin, “Genes V” (Oxford University Press, Oxford) pages 847-873). Exemplary regulatory elements in prokaryotes include promoters, operator sequences and a ribosome binding sites. Regulatory elements that are used in eukaryotic cells can include, without limitation, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell.

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “enhancer” refers to a type of regulatory element that can increase the efficiency of transcription, regardless of the distance or orientation of the enhancer relative to the start site of transcription.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

### Molecular Recording Systems

Understanding how a single cell can generate a multicellular organism is a fundamental goal of biology. Reconstructing the familial branches between related cells can reveal how lineage dictates cell fate. The ability to reconstruct the branching family histories of cell populations enables identification of correlations between cell states and lineage, however, determining how different cells are related becomes challenging to impossible in large, opaque organisms. Provided herein are two phylogenetic recording systems for high resolution lineage reconstruction over long time scales. The first system, termed bigMEMOIR (e.g., baseMEMOIR) can generate heritable mutations in synthetic A-to-G base editor (ABE) targets designed for in situ readout. These targets were integrated at high copy number into mouse embryonic stem cells (mESCs), then this cell line was used to recover lineage relationships in colonies grown in vitro (See, e.g., Examples 1 and 3). The second system, termed the hypercascade, takes advantage of the predictability of the ABE to generate new editable targets over time as old targets are used up. This process linearizes the overall rate of edit accumulation within the cell, extending the effective recording window relative to non-regenerating target arrays. Lineage relationships in human induced pluripotent stem cells (hiPSCs) could be recorded over a period of several weeks (See, e.g., Example 2). Hypercascade can take advantage of the CRISPR A-to-G base editor (ABE) to produce edits at a constant rate over time within the cell, widening the effective recording window. Both recording systems provided herein enable long-term lineage recording at high resolution. These systems can be applied to better understand cellular differentiation in vivo and in vitro. Systems provided herein can be integrated into cells (e.g., mouse embryonic stem cells (mESCs)). These cell lines can be employed to, e.g., recover lineage relationships in small mESC colonies grown in vitro. Further, these cells can be integrated into developing synthetic mouse embryos to examine the relationships between fate commitment and spatial proximity, e.g., during a model of gastrulation.

There are provided, in some embodiments, systems. In some embodiments, the system comprises: (i) one or more hypercascade array(s) each comprising p hypercascade units, wherein p is an integer greater than 1; (ii) n layer guide RNAs (gRNAs), wherein n is an integer greater than 1; and/or (iii) an editor. The editor can be a base editor capable of base editing the hypercascade array. In some embodiments, said base editing comprises: adenine (A)-to-guanine (G) base editing and/or cytosine (C)-to-thymine (T) base editing. Each hypercascade unit can comprise m target sites, and m can be an integer greater than 1. The target sites can comprise one or more primary target site(s) and one or more conditional target site(s). The target sites each can comprise an editable base. The editor associated with a layer gRNA can be capable of editing said editable base of a target site comprising a complementary protospacer and a protospacer adjacent motif (PAM). The primary target sites can be capable of being edited by a first layer gRNA and the editor. Each of the one or more conditional target sites can comprise two or more mismatches in the protospacer and/or the PAM. In some embodiments, the editing of adjacent editable bases by a previous layer gRNA are capable of repairing said mismatches, thereby enabling the editing of a conditional target site by a layer gRNA and the editor.

The one or more hypercascade array(s) can comprise at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 hypercascade arrays. Each hypercascade unit can comprise the same length and/or sequence. Each hypercascade unit can comprise an identical N-mer. The N-mer can be 15, 16, 17, 18, 19, 20, 21, 22, 23, 24, 25, 26, 27, 28, 29, 30, 31, 32, 33, 34, 35, 36, 37, 38, 39, or 40, nucleotides (nt) in length. The N-mer can be 20 nt in length. The two or more of the p hypercascade units can be in tandem. The hypercascade array can comprise a tandem repeating 20-mer. The hypercascade unit can comprise a nucleotide sequence that is at least 80%, 85%, 90%, 95%, 98%, 99%, or 100% identical to (NGGNNAGNNAGNNAGNNANN). The hypercascade unit can comprise a nucleotide sequence that is at least 80%, 85%, 90%, 95%, 98%, 99%, or 100% identical to SEQ ID NO: 1 (AGGACAGTCAGACAGTCATG). The hypercascade unit can comprise a nucleotide sequence that is at least 80%, 85%, 90%, 95%, 98%, 99%, or 100% identical to SEQ ID NO: 2 (AGGTCAGACAGTCAGACACA). The hypercascade unit can comprise a nucleotide sequence that is at least 80%, 85%, 90%, 95%, 98%, 99%, or 100% identical to SEQ ID NO: 3 (AGGTCAGTCAGTAAGTAACG). The PAM can be about 2-6 nucleotides in length (e.g., 3nucleotides in length). The PAM can comprise NGG.

Each hypercascade unit can comprise m−1 conditional target sites capable of being activated by adjacent edits mediated by an upper layer gRNA. The editable base can be situated at the fifth or sixth position of a target site. The editable base can comprise adenine, optionally edited to guanine by the editor. In some embodiments, the repair of repairing protospacer and PAM mismatches through A-to-G edits of a previous layer gRNA enable the editing of conditional target sites. In some embodiments, the n layer gRNAs comprise: a first layer gRNA; a second layer gRNA; a third layer gRNA; and/or a fourth layer gRNA. The first layer gRNA associated with the editor can be capable of editing the primary target sites. The second layer gRNA associated with the editor can be capable of editing first conditional target sites upon repair of adjacent mismatches by the first layer gRNA associated with the editor. The third layer gRNA associated with the editor can be capable of editing second conditional target sites upon repair of adjacent mismatches by the second layer gRNA associated with the editor. The fourth layer gRNA associated with the editor can be capable of editing third conditional target sites upon repair of adjacent mismatches by the third layer gRNA associated with the editor. The first layer gRNA can comprise a nucleotide sequence that is at least 80%, 85%, 90%, 95%, 98%, 99%, or 100% identical to (NGGNNAGNNAGNNAGNNANN). The second layer gRNA can comprise a nucleotide sequence that is at least 80%, 85%, 90%, 95%, 98%, 99%, or 100% identical to (NGGNNAGNNAGNNANNNGGN). The third layer gRNA can comprise a nucleotide sequence that is at least 80%, 85%, 90%, 95%, 98%, 99%, or 100% identical to (NGGNNAGNNANNNGGNNGGN). The fourth layer gRNA can comprise a nucleotide sequence that is at least 80%, 85%, 90%, 95%, 98%, 99%, or 100% identical to (NGGNNANNNGGNNGGNNGGN). The layer gRNA can be a single guide RNA (sgRNA).

In some embodiments, each hypercascade array comprises: a first static barcode; and/or one or more second static barcodes (e.g., two second static barcodes). The first static barcode can be about 10 nt in length. The first static barcode can be selected from a library of at least about 106 different first static barcode sequences. The one or more second static barcodes can be image-readable. A second static barcode can be selected from a library of at least about 200 different second static barcode sequences.

In some embodiments, upon introduction into a cell, the system achieves extended durations of editing with near-linear barcode loss over a period of time (e.g., at least about 2, 3, 4, 5, 6, 7, 8, 9, 10, 11, 12, 13, 14, 15, 16, 17, 18, 19, or 20, generations). In some embodiments, the relative insensitivity of the system to edit rate and hypercascade unit copy number enables deep lineage reconstruction (e.g., in polyclonal populations of cells). The editor can be selected from the group comprising CRISPR-Cas9, base editors, prime editors, integrases, and recombinases. The editor can be a base editor capable of base editing the hypercascade unit. In some embodiments, said base editing comprises: adenine (A)-to-guanine (G) base editing and/or cytosine (C)-to-thymine (T) base editing. The base editor can comprise saCas9-KKH, Cas9-VQR, Cas9-VRQR, Cas9-VRER, Cas9-NG, ABE7.7, pNMG-624, ABE3.2, ABE5.3, pNMG-558, pNMG-576, pNMG-577, pNMG-586, ABE7.2, pNMG-620, pNMG-617, pNMG-618, pNMG-620, pNMG-621, pNGM-622, pNMG-623, ABE6.3, ABE6.4, ABE7.8, ABE7.9, ABE7.10, ABEMax, ABE8e, CP1028-ABE8e, ABE7.10-CP1041, CP1041-ABE8e, or any combination thereof. The base editor can comprise an adenine base editor (ABE) and/or a cytosine base editor (CBE). The ABE can comprise monomer and dimer versions of one or more of ABE8e, ABE8e-V106W, SaABE8e, SaKKH-ABE8e, NG-ABE8e, ABE-xCas9, ABE8e-NRTH, ABE8e-NRRH, ABE8e-NRCH, ABE8e-NG-CP1041, ABE8e-VRQR-CP1041, ABE8e-CP1041, ABE8e-CP1028, ABE8e-VRQR, ABE8e-LbCas12a (LbABE8e), ABE8e-AsCas12a (enAsABE8e), ABE8e-SpyMac, ABE8e (TadA-8e V106W), ABE8e (K20A,R21A), and ABE8e (TadA-8e V82G).

In some embodiments, p is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. In some embodiments, n is 2, 3, 4, 5, 6, 7, 8, 9, 10, 11, 12, 13, 14, or 15. In some embodiments, n=m. In some embodiments, m is 2, 3, 4, 5, 6, 7, 8, 9, 10, 11, 12, 13, 14, or 15. In some embodiments, one or more polynucleotides encoding components of the system are capable of being introduced into a cell in a single step (e.g., via piggyBac transposition). The length of the hypercascade array can be at least, or most, about 200 bp, 400 bp, 600 bp, 800 bp, 1.0 kb, 1.5 kb, 2.0 kb, 2.5 kb, 3.0 kb, 3.5 kb, 4.0 kb, 4.5 kb, 5.0 kb, 5.5 kb, 6.0 kb, 6.5 kb, 7.0 kb, or 7.5 kb. In some embodiments, the system capable of linear editing for an increased period before saturation as compared to non-regenerative target arrays. The hypercascade array can comprise an internal priming site capable of binding a custom sequencing primer, thereby enabling recovery of the entire hypercascade array along with the first and/or second static barcode(s) via a short-read sequencing protocol (e.g., via 3′ gene expression protocol).

There are provided, in some embodiments, nucleic acid compositions. In some embodiments, the nucleic acid composition comprises: one or more first polynucleotide(s) encoding one or more hypercascade arrays disclosed herein; one or more second polynucleotide(s) encoding n layer guide RNAs (gRNAs) disclosed herein; and/or one or more third polynucleotide(s) encoding an editor disclosed herein. One or more of the first polynucleotide(s), the second polynucleotide(s), and the third polynucleotide(s) can be operably connected to a promoter selected from the group comprising: an RNA pol I promoter; a pol II promoter (e.g., CMV, SV40 early region or adenovirus major late promoter); a pol III promoter (e.g., a U6 or H1 promoter); a minimal promoter (e.g., TATA, miniCMV, and/or miniPromo); a bacteriophage promoter (e.g., a bacteriophage T3 promoter, a bacteriophage T7 promoter, a bacteriophage SP6 promoter, or a combination thereof); a tissue-specific promoter; a lineage-specific promoter; and/or a ubiquitous promoter (e.g., a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CASI promoter, a CBH promoter, or any combination thereof).

The nucleic acid composition can be complexed or associated with one or more lipids or lipid-based carriers, thereby forming liposomes, lipid nanoparticles (LNPs), lipoplexes, and/or nanoliposomes (e.g., encapsulating the nucleic acid composition). In some embodiments, the nucleic acid composition is, comprises, or further comprises, one or more vectors. At least one of the one or more vectors can be a viral vector, a plasmid, a transposable element, a naked DNA vector, a lipid nanoparticle (LNP), or any combination thereof. The viral vector can be an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof. The transposable element can be piggybac transposon or sleeping beauty transposon. The target array(s) can be flanked with piggyBac inverted terminal repeats. A T7 promoter can be situated upstream of the target array(s). The first polynucleotide(s), the second polynucleotide(s), and/or the third polynucleotide(s) can be comprised in the one or more vectors. The first polynucleotide(s), the second polynucleotide(s), and/or the third polynucleotide(s) can be comprised in the same vector and/or different vectors. The first polynucleotide(s), the second polynucleotide(s), and/or the third polynucleotide(s) can be situated on the same nucleic acid and/or different nucleic acids. The hypercascade array(s) can be flanked with piggyBac inverted terminal repeats.

There are provided, in some embodiments, populations of cells. In some embodiments, the population of cells comprises: one or more hypercascade array(s) disclosed herein; n layer guide RNAs (gRNAs) disclosed herein; and/or an editor disclosed herein. In some embodiments, the population of cells comprises: one or more first polynucleotides encoding one or more hypercascade array(s) disclosed herein; one or more second polynucleotides encoding n layer guide RNAs (gRNAs) disclosed herein; and/or one or more third polynucleotides encoding an editor disclosed herein. The population of cells can comprise at least about 10, 100, 1000, 10000, 25000, 50000, 75000, 100000, 500000, or 1000000 cells.

The population of cells can comprise one or more of the following: a primary cell, an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. The stem cell can comprise an embryonic stem cell, an induced pluripotent stem cell (iPSC), a hematopoietic stem/progenitor cell (HSPC), or any combination thereof.

There are provided, in some embodiments, methods. In some embodiments, the method comprises: introducing the one or more hypercascade array(s) disclosed herein, the n layer guide RNAs (gRNAs) disclosed herein, and the editor disclosed herein into a cell or a first population of cells; incubating the cell(s) for a period of time; and/or obtaining sequence information of the hypercascade array(s) of each of the resulting second population of cells (e.g., via sequencing or imaging). The introducing step can comprise introducing a nucleic acid composition disclosed herein into the cell(s).

In some embodiments, the cell(s) undergo at least about 2, 3, 4, 5, 6, 7, 8, 9, 10, 11, 12, 13, 14, 15, 16, 17, 18, 19, or 20, generations during the incubation. The period of time can comprise at least about 6 hr, 12 hr, 18 hr, 24 hr, 1 day, 2 days, 3 days, 4 days, 5 days, 6 days, 7 days, 10 days, 2 weeks, 3 weeks, or 4 weeks. The method further can comprise generating a transcriptomic profile of each of the resulting second population of cells. The method can comprise reconstructing lineage relationships between the resulting second population of cells. In some embodiments, edits accumulate linearly over a period of time of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days. In some embodiments, the first polynucleotide(s), the second polynucleotide(s), and/or the third polynucleotide(s) become integrated in the genome of the population of cell(s) after the introducing step. In some embodiments, two or more hypercascade arrays can be integrated into a genome of at least one cell, and different integrations can be capable of being distinguished from one another via the first static barcode and/or second static barcode(s).

The method can comprise exposing the cell(s) to one or more agents. In some embodiments, the one or more agents comprise: one or more of a chemical agent, a pharmaceutical, small molecule, a biologic, a CRISPR single-guide RNA (sgRNA), a small interfering RNA (siRNA), CRISPR RNA (crRNA), a small hairpin RNA (shRNA), a microRNA (miRNA), a piwi-interacting RNA (piRNA), an antisense oligonucleotide, a peptide or peptidomimetic inhibitor, an aptamer, an antibody, an intrabody, or any combination thereof; an expression vector, wherein the expression vector encodes one or more of the following: an mRNA, an antisense nucleic acid molecule, a RNAi molecule, a shRNA, a mature miRNA, a pre-miRNA, a pri-miRNA, an anti-miRNA, a ribozyme, any combination thereof; an infectious agent, an anti-infectious agent, or a mixture thereof; a cytotoxic agent, optionally a chemotherapeutic agent, a biologic agent, a toxin, a radioactive isotope, or any combination thereof; one or more of an epigenetic modifying agent, epigenetic enzyme, a bicyclic peptide, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis inhibitor, a nuclease, a protein fragment or domain, a tag or marker, an antigen, an antibody or antibody fragment, a ligand or a receptor, a synthetic or analog peptide from a naturally-bioactive peptide, an anti-microbial peptide, a pore-forming peptide, a targeting or cytotoxic peptide, a degradation or self-destruction peptide, a CRISPR component system or component thereof, DNA, RNA, artificial nucleic acids, a nanoparticle, an oligonucleotide aptamer, a peptide aptamer, or any combination thereof; and/or at least one effector activity selected from the group consisting of: modulating a biological activity, binding a regulatory protein, modulating enzymatic activity, modulating substrate binding, modulating receptor activation, modulating protein stability/degradation, modulating transcript stability/degradation, or any combination thereof.

There are provided, in some embodiments, systems. In some embodiments, the system comprises: (i) one or more target array(s) comprising n editable target sites, wherein n is an integer greater than 1; (ii) n guide RNAs (gRNAs); and/or (iii) a base editor capable of adenine (A)-to-guanine (G) base editing. Each guide RNA of the n gRNAs can comprise a unique spacer sequence capable of binding one of the n editable target sites. Each editable target site of the n editable target sites can comprise an AA dinucleotide and a unique protospacer sequence relative to the other n editable target sites. The base editor associated with one of the n gRNAs can be capable of editing the AA dinucleotide to a GA dinucleotide, AG dinucleotide, or GG dinucleotide. In some embodiments, a GA dinucleotide and an AG dinucleotide each disrupt binding to the gRNA, and thereby edited target sites comprising a GA dinucleotide and an AG dinucleotide are incapable of being further edited to a GG dinucleotide.

The base editor can comprise an adenine base editor (ABE). The ABE can comprise monomer and dimer versions of one or more of ABE8e, ABE8e-V106W, SaABE8e, SaKKH-ABE8e, NG-ABE8e, ABE-xCas9, ABE8e-NRTH, ABE8e-NRRH, ABE8e-NRCH, ABE8e-NG-CP1041, ABE8e-VRQR-CP1041, ABE8e-CP1041, ABE8e-CP1028, ABE8e-VRQR, ABE8e-LbCas12a (LbABE8e), ABE8e-AsCas12a (enAsABE8e), ABE8e-SpyMac, ABE8e (TadA-8e V106W), ABE8e (K20A,R21A), ABE8e (TadA-8e V82G), ABE7.7, pNMG-624, ABE3.2, ABE5.3, ABE7.2, pNMG-620, pNMG-617, pNMG-618, pNMG-620, pNMG-621, pNGM-622, pNMG-623, ABE6.3, ABE6.4, ABE7.8, ABE7.9, ABE7.10, ABEMax, ABE8e, CP1028-ABE8e, ABE7.10-CP1041, CP1041-ABE8e, or any combination thereof.

In some embodiments, n is 2, 3, 4, 5, 6, 7, 8, 9, 10, 11, 12, 13, 14, 15, 16, 17, 18, 19, or 20. The n editable target sites can be in tandem. The editable target site can be about 20 bp to about 40 bp in length. The one or more target array(s) can comprise at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 75, 80, 85, 90, 95, 100, 105, 110, 115, 120, 125, 130, 135, 140, 145, 150, 155, 160, 165, 170, 175, 180, 185, 190, 195, 200 target arrays. In some embodiments, each target array comprises: a first static barcode; and/or one or more second static barcodes (e.g., two second static barcodes). The first static barcode can be about 10 nt in length. The first static barcode can be selected from a library of at least about 10⁶ different first static barcode sequences. The one or more second static barcodes can be image-readable. A second static barcode can be selected from a library of at least about 200 different second static barcode sequences.

The gRNA can be a single guide RNA (sgRNA). The expression of the n guide RNAs can be under the control of a first inducible promoter. The first inducible promoter can be capable of inducing transcription in the presence of a first agent. The first inducible promoter can comprise a Wnt-responsive element (WRE). The first agent can be a Wnt signaling ligand (e.g., GSK-3 inhibitor CHIR99021 (CHIR)). The expression of the base editor can be under the control of a second inducible promoter. The second inducible promoter can be capable of inducing transcription in the presence of a second agent. The second agent can comprise tetracycline, doxycycline or a derivative thereof. The inducible second promoter can comprise one or more copies of a transactivator recognition sequence a transactivator can be capable of binding to induce transcription. The transactivator can be incapable of binding the transactivator recognition sequence in the absence of a transactivator-binding compound. The one or more copies of a transactivator recognition sequence can comprise one or more copies of a tet operator (TetO). The second agent can comprise the transactivator-binding compound. In some embodiments, one or more polynucleotides encoding components of the system are capable of being introduced into a cell in a single step (e.g., via piggyBac transposition).

There are provided, in some embodiments, nucleic acid compositions. In some embodiments, the nucleic acid composition comprises: one or more first polynucleotide(s) encoding the one or more target array(s) disclosed herein; one or more second polynucleotide(s) encoding the n guide RNAs (gRNAs) disclosed herein; and/or one or more third polynucleotide(s) encoding the base editor disclosed herein. The one or more second polynucleotide(s) can be operably connected to a first inducible promoter and the one or more third polynucleotide(s) can be operably connected to a second inducible promoter.

In some embodiments, one or more of the first polynucleotide(s), the second polynucleotide(s), and the third polynucleotide(s) can be operably connected to a promoter selected from the group comprising: an RNA pol I promoter; a pol II promoter (e.g., CMV, SV40 early region or adenovirus major late promoter); a pol III promoter (e.g., a U6 or H1 promoter); a minimal promoter (e.g., TATA, miniCMV, and/or miniPromo); a bacteriophage promoter (e.g., a bacteriophage T3 promoter, a bacteriophage T7 promoter, a bacteriophage SP6 promoter, or a combination thereof); a tissue-specific promoter; a lineage-specific promoter; and/or a ubiquitous promoter (e.g., a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (B-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CASI promoter, a CBH promoter, or any combination thereof).

The nucleic acid composition can be complexed or associated with one or more lipids or lipid-based carriers, thereby forming liposomes, lipid nanoparticles (LNPs), lipoplexes, and/or nanoliposomes (e.g., encapsulating the nucleic acid composition). In some embodiments, the nucleic acid composition is, comprises, or further comprises, one or more vectors. At least one of the one or more vectors can be a viral vector, a plasmid, a transposable element, a naked DNA vector, a lipid nanoparticle (LNP), or any combination thereof. The viral vector can be an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof. The transposable element can be piggybac transposon or sleeping beauty transposon. The target array(s) can be flanked with piggyBac inverted terminal repeats. A T7 promoter can be situated upstream of the target array(s). The first polynucleotide(s), the second polynucleotide(s), and/or the third polynucleotide(s) can be comprised in the one or more vectors. The first polynucleotide(s), the second polynucleotide(s), and/or the third polynucleotide(s) can be comprised in the same vector and/or different vectors. The first polynucleotide(s), the second polynucleotide(s), and/or the third polynucleotide(s) can be situated on the same nucleic acid and/or different nucleic acids.

There are provided, in some embodiments, populations of cells. In some embodiments, the population of cells comprises: one or more target array(s) disclosed herein; n guide RNAs (gRNAs) disclosed herein; and/or a base editor disclosed herein. In some embodiments, the population of cells comprises: one or more first polynucleotide(s) encoding one or more target array(s) disclosed herein; one or more second polynucleotide(s) encoding n guide RNAs (gRNAs) disclosed herein; and/or one or more third polynucleotide(s) encoding a base editor disclosed herein. The cells can comprise integrated reverse tetracycline-controlled transactivator (rtTA). The population of cells can comprise at least about 10, 100, 1000, 10000, 25000, 50000, 75000, 100000, 500000, or 1000000 cells.

The population of cells can comprise one or more of the following: a primary cell, an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. The stem cell can comprise an embryonic stem cell, an induced pluripotent stem cell (iPSC), a hematopoietic stem/progenitor cell (HSPC), or any combination thereof.

There are provided, in some embodiments, methods. In some embodiments, the method comprises: introducing one or more target array(s) disclosed herein, n guide RNAs (gRNAs) disclosed herein, and a base editor disclosed herein into a cell or a first population of cells; incubating the cell(s) for a period of time; and/or obtaining sequence information of the one or more target array(s) of each of the resulting second population of cells (e.g., via sequencing or imaging). The introducing step can comprise introducing a nucleic acid composition disclosed herein into the cell(s). The method can comprise contacting the cells with the first agent and/or the second agent to induce expression of the n guide RNAs and/or the base editor, respectively. In some embodiments, imaging can comprise single molecule RNA FISH (smFISH). The method can comprise fixing the resulting second population of cells and in situ T7 transcription.

In some embodiments, the cell(s) undergoes at least about 2, 3, 4, 5, 6, 7, 8, 9, 10, 11, 12, 13, 14, 15, 16, 17, 18, 19, or 20, generations during the incubation. The period of time can comprise at least about 6 hr, 12 hr, 18 hr, 24 hr, 1 day, 2 days, 3 days, 4 days, 5 days, 6 days, 7 days, 10 days, 2 weeks, 3 weeks, or 4 weeks. The method further can comprise generating a transcriptomic profile of each of the resulting second population of cells. The method can comprise reconstructing lineage relationships between the resulting second population of cells. In some embodiments, lineage can be accurately reconstructed for at least about 4, 5, 6, 7, 8, 9, 10, 11, or 12 generations. The method can comprise deriving correlations between lineage history, spatial position, and cell fate. The method can comprise combined the lineage reconstruction with spatial and cell state dynamics to infer the relative spatial mobilities of different cell states. In some embodiments, edits accumulate over a period of time of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days. In some embodiments, the first polynucleotide(s), the second polynucleotide(s), and/or the third polynucleotide(s) become integrated in the genome of the cell(s) after the introducing step. The two or more target array(s) can be integrated into a genome of at least one cell, and the different integrations can be capable of being distinguished from one another via the first static barcode and/or second static barcode(s). The method can comprise exposing the cell(s) to one or more agents.

In some embodiments, the one or more agents comprise: one or more of a chemical agent, a pharmaceutical, small molecule, a biologic, a CRISPR single-guide RNA (sgRNA), a small interfering RNA (siRNA), CRISPR RNA (crRNA), a small hairpin RNA (shRNA), a microRNA (miRNA), a piwi-interacting RNA (piRNA), an antisense oligonucleotide, a peptide or peptidomimetic inhibitor, an aptamer, an antibody, an intrabody, or any combination thereof; an expression vector, wherein the expression vector encodes one or more of the following: an mRNA, an antisense nucleic acid molecule, a RNAi molecule, a shRNA, a mature miRNA, a pre-miRNA, a pri-miRNA, an anti-miRNA, a ribozyme, any combination thereof; an infectious agent, an anti-infectious agent, or a mixture thereof; a cytotoxic agent, optionally a chemotherapeutic agent, a biologic agent, a toxin, a radioactive isotope, or any combination thereof; one or more of an epigenetic modifying agent, epigenetic enzyme, a bicyclic peptide, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis inhibitor, a nuclease, a protein fragment or domain, a tag or marker, an antigen, an antibody or antibody fragment, a ligand or a receptor, a synthetic or analog peptide from a naturally-bioactive peptide, an anti-microbial peptide, a pore-forming peptide, a targeting or cytotoxic peptide, a degradation or self-destruction peptide, a CRISPR component system or component thereof, DNA, RNA, artificial nucleic acids, a nanoparticle, an oligonucleotide aptamer, a peptide aptamer, or any combination thereof; and/or at least one effector activity selected from the group consisting of: modulating a biological activity, binding a regulatory protein, modulating enzymatic activity, modulating substrate binding, modulating receptor activation, modulating protein stability/degradation, modulating transcript stability/degradation, or any combination thereof.

In some embodiments of the systems provided therein, an additional layer of barcoding (e.g., static barcoding) is employed to enable multiplexing of hypercascade targets in single cells (enabling many copies of the array could be simultaneously integrated and distinguished by one another). This can increase the total information stored in a cell to allow for longer and more detailed lineage reconstruction. Barcode arrays provided herein can be integrated at high copy number into cells (e.g., mouse embryonic stem cells) using piggyBac transposition. In some embodiments, barcodes provided herein can be recovered from single cells by modifying the 10× Genomics 3′ gene expression protocol, followed by sequencing the entire array using short read sequencing. A similar approach is described in Simeonov, Kamen P., et al. (“Single-cell lineage tracing of metastatic cancer reveals selection of hybrid EMT states.” Cancer cell 39.8 (2021): 1150-1162) though modified herein to address the challenge of reading out all necessary parts of the disclosed barcodes due to sequence length by including an internal priming site in the hypercascade construct design that allows for a custom sequencing primer to bind and recover the entire array along with the static barcode during short-read sequencing.

Disclosed herein include kits. Kits can comprise one or more components of the systems provided herein. The systems, methods, compositions, and kits provided herein can, in some embodiments, be employed in concert with the systems, methods, compositions, and kits described in PCT Application Publication No. WO2020117713A1, entitled, “In situ readout of dna barcodes,” the content of which is incorporated herein by reference in its entirety. The systems, methods, compositions, and kits provided herein can, in some embodiments, be employed in concert with the systems, methods, compositions, and kits described in U.S. patents application Ser. Nos. 17/820,232, 17/820,235, and 18/757,460, the contents of which are incorporated herein by reference in their entiret. The systems, methods, compositions, and kits provided herein can, in some embodiments, be employed in concert with the systems, methods, compositions, and kits described in Chadly, Duncan M., et al. (“Reconstructing cell histories in space with image-readable base editor recording.” bioRxiv (2024): 2024-01; which can be retrieved at biorxiv.org/content/10.1101/2024.01.03.573434v1.full), the content of which is incorporated herein by reference in its entirety. In particular, the supplementary materials and supplemental data of said Chadly et al. reference (which can be retrieved at biorxiv.org/content/10.1101/2024.01.03.573434v1.supplementary-material), which includes Supplemental Movies, Primary Probe Sequences, Readout Probe Sequences, and Static Barcode Library Sequences, hybridization rounds for imaging experiments, baseMEM-01_WRE gRNAs sequences, baseMEM-01_tetOn-ABE sequences, and baseMEM-01_target_array sequences, is incorporated herein by reference in its entirety. An exemplary FISHable static barcode sequences which can be employed with the baseMEMOIR target array is SEQ ID NO: 17 (TGTAGTGAAGAGAACGACCCGATGTTACCACCCCAAGAGAGATACAACTCCAACTGTT CCCCAGTTACCATCCCAAGCCT).

### EXAMPLES

Some aspects of the embodiments discussed above are disclosed in further detail in the following examples, which are not in any way intended to limit the scope of the present disclosure.

### Example 1

### Reconstructing Cell Histories in Space with Image-Readable Base Editor Recording

Knowing the ancestral states and lineage relationships of individual cells could unravel the dynamic programs underlying development. Engineering cells to actively record information within their own genomic DNA could reveal these histories, but existing recording systems have limited information capacity or disrupt spatial context. BaseMEMOIR is provided herein, which combined base editing, sequential hybridization imaging and Bayesian inference to allow reconstruction of high-resolution cell lineage trees and cell state dynamics while preserving spatial organization. BaseMEMOIR stochastically and irreversibly edits engineered dinucleotides to one of three alternative image-readable states. By genomically integrating arrays of editable dinucleotides, an embryonic stem cell line with 792 bits of recordable, image-readable memory, and a 50-fold increase over the state of the art, was constructed. Simulations showed that this memory size was sufficient for accurate reconstruction of deep lineage trees. Experimentally, baseMEMOIR allowed precise reconstruction of lineage trees 6 or more generations deep in embryonic stem cell colonies. Further, it also allowed inference of ancestral cell states and their quantitative cell state transition rates, all from endpoint images. Thus, baseMEMOIR provides a scalable framework for reconstructing single cell histories in spatially organized multicellular systems.

### Introduction

Cells divide, differentiate, and move to form exquisitely organized structures. Reconstructing the dynamic histories of individual cells, particularly their lineage relationships, could enable researchers to understand how tissues form, analyze the roles of intrinsic and extrinsic determinants of cell fate decisions, and reveal how processes are dysregulated in disease. Recent advances in single cell sequencing and spatial genomics now allow us to capture single cell states at specific moments in time. However, with a few exceptions, the histories of those cells have largely remained hidden.

Researchers have sought to address this challenge through engineered recording systems, which progressively introduce stochastic edits in genomically integrated barcode sequences as cells proliferate. Systems such as GESTALT, CARLIN, LINNAEUS, SMALT and the homing CRISPR barcoded mouse, use CRISPR/Cas9 or recombinases to edit designed target sequences, relying on next generation sequencing to read out edited barcodes. Alternative systems, including CAMERA, leverage CRISPR base editors to generate more specific types of barcode diversity. Furthermore, prime editors introduced an additional paradigm for phylogenetic recording, sequentially inserting short nucleotide motifs for genomic information storage. In all of these systems, lineage relationships between individual cells are reconstructed from each cell's unique pattern of target site edits, in a manner analogous to sequence-based phylogenetic reconstruction.

These techniques can be powerful in their ability to recover lineage. However, all of these techniques but disrupt spatial organization. A parallel set of methods were developed to allow barcode editing in ways that allow readout of edits and cell states by imaging. For example, previous MEMOIR (Memory by Engineered Mutagenesis with Optical in situ Readout) systems showed that it is possible to stochastically and irreversibly edit engineered DNA barcodes, or ‘scratchpads,’ using CRISPR/Cas9 or an integrase, and then read out those edits by imaging. However, these methods were limited in accuracy by relatively low numbers of mutable target sites, which serve as memory in the genome. For example, existing image-readable systems have demonstrated only ˜16 bits of information storage.

It has been shown that in situ T7 transcription can amplify genomic DNA into localized RNA clusters, which can then be competitively probed to discriminate single base edits. In this strategy, termed “Zombie,” a genomic DNA of interest can be maintained without transcription in live cells, transcribed after fixation with the addition of T7 polymerase, and finally detected by RNA-FISH. Zombie transcription avoids silencing problems that occur when barcodes must be continuously transcribed in live cells, generates large quantities of RNA that spatially localize around the active site of transcription, can detect mutations at single nucleotide resolution, and is compatible with subsequent sequential rounds of FISH to detect endogenous gene transcripts. Zombie enables readout of dense editable memory arrays, expanding the capacity of MEMOIR approaches.

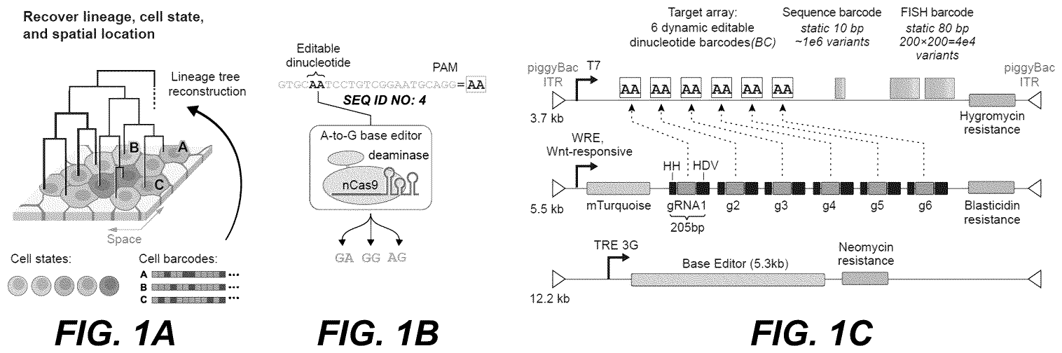

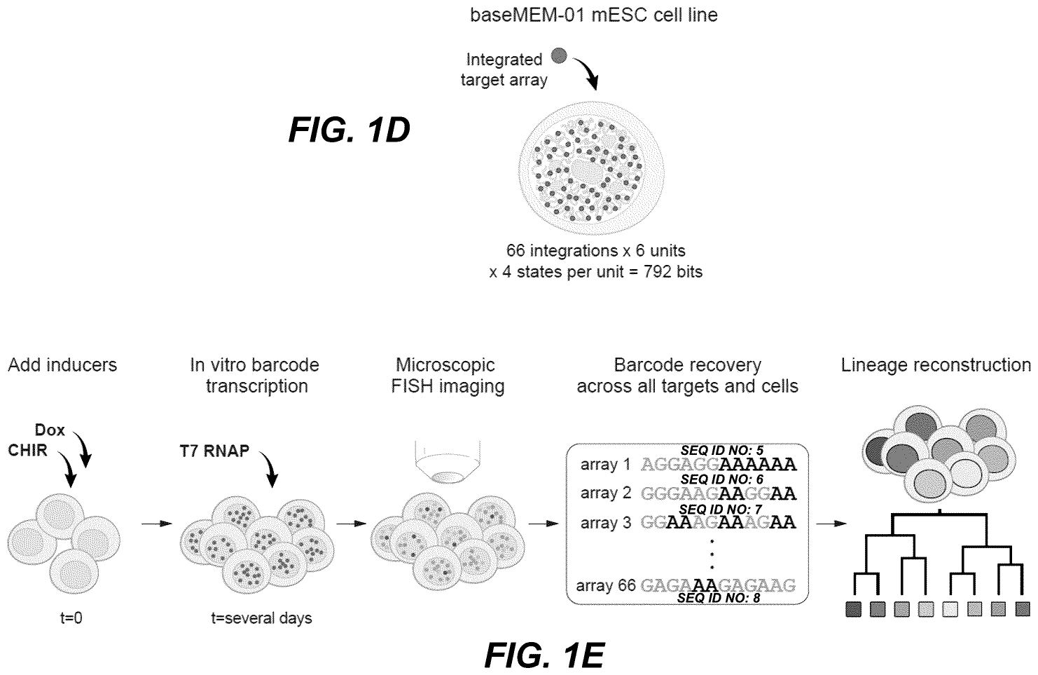

Disclosed herein includes “baseMEMOIR,” a multiplexed phylogenetic recording system, which enables detailed recording of lineage relationships over time in a manner compatible with recovery of spatial position and gene expression patterns (FIG. 1A). Mutable synthetic DNA sequences were distributed at high copy number randomly across the genome of mouse embryonic stem cells. These targets (e.g., the mutable synthetic DNA sequences) can be edited by the CRISPR A-to-G base editor (ABE), which complexed with guide RNAs to specifically mutate target sites within the synthetic sequences. For tight control of editing, two inducible systems were used to control the base editor (e.g., the TRE3G Tet-On system) and guide RNAs (e.g., guide RNAs controlled by a Wnt responsive promoter) respectively. On induction, mutations occurred at a rate commensurate with cell division and were passed down from parent cells to their progeny through DNA replication, creating lasting marks that link related cells to one another. Mutation states were then recovered through microscopic imaging, and Bayesian phylogenetic tools were applied to infer lineage relationships as well as transcriptional state dynamics and spatiotemporal histories in a unified manner. By comparing the distinct pattern of mutations in each cell after a series of divisions, phylogenies can be reconstructed and uncertainty can be estimated in both tree topology and the timing of past cell divisions.

To demonstrate the capabilities of baseMEMOIR, the system disclosed herein was applied to estimate state switching rates and probable past cell states along lineages of dividing mESCs grown in serum-LIF conditions in the presence of a Wnt agonist. As a result, mESCs grown in these conditions underwent reversible transitions between formative and 2C-like states, with an intermediate naive state that can be broken up into three distinct subclusters. Each state and subcluster, in addition to being transcriptionally distinct, was further distinguished by a set of allowed state transitions. The baseMEMOIR cell lines and platform can be applied, and further scaled, in any model system permitting genetic engineering. Thus, baseMEMOIR enables spatially resolved analysis of embryonic development and other processes.

### Results

### Base Editing can Enable Lineage Recording with Spatial Readout

To ultimately capture detailed lineage relationships between cells while maintaining spatial context, an image-readable stochastic base editing system was designed. In some embodiments, such a system employs designed target sequences that would be editable at a single base. For example, the A-to-G base editor (ABE) could target a set of defined sequences to stochastically edit each target site. However, this scheme was susceptible to convergent edits in unrelated cells (e.g., homoplasy). For example, every editable A would be converted to a G in all cells in the situation of complete editing, and no lineage information could be recovered.

To circumvent this issue, a modified design was used, which takes advantage of the ability of the ABE to stochastically edit target sequences into one of multiple stable outcomes. For example, AA dinucleotide sequences in the target window are converted to any of three edited end-point states (e.g., GA, AG or GG) (FIG. 1B). Critically, because each of the GA and AG states each disrupt binding to the base editor gRNA, they are not expected to undergo further editing to GG. This dinucleotide editing scheme in principle reduces the likelihood of convergent editing, and prevents the effective “erasure” of recorded information at long times.

Based on this principle, a library of barcoded, editable target arrays that could be integrated into the genome (FIG. 1C) was designed. Each target array contained 6 tandem editable target sites, with unique protospacer sequences outside of the AA dinucleotide, so that each of the 6 target sites required a distinct gRNA sequence for editing (FIG. 1C). The arrays were flanked with piggyBac inverted terminal repeats, to enable high-copy-number genomic integration. To distinguish different integrations from one another, two static (non-editable) random barcodes were also incorporated, including: first, a 10 bp barcode (10⁶ variants), for compact readout by sequencing; second, a pair of static 80 bp image-readable barcode sequences, each of which could take on one of 200 possible sequences, for a total diversity of 200²=˜10⁴ unique barcodes. Finally, to enable imaging-based “Zombie” readout of edits, the arrays incorporated a T7 promoter upstream of the editable array (FIG. 1C).

To mutate targets at a tunable rate, and create the potential for signal recording, constructs that allow inducible expression of the ABE and gRNAs were built. ABE expression was placed under the control of doxycycline (dox) using the Tet-ON system, by stably integrating the reverse tetracycline-controlled transactivator (rtTA). gRNAs were also made Wnt-inducible by expressing them from the 3′ UTR of an mTurquoise reporter gene under the control of a Wnt-responsive element (WRE). This promoter (e.g., WRE) is active only in the presence of Wnt signaling ligands, and can be activated by the small molecule GSK-3 inhibitor CHIR99021 (CHIR). To generate fully functional gRNAs after expression, each gRNA was flanked with ribozyme sequences described in a previous report. After stable co-integration in mESCs using piggyBac transposition, a clone, termed baseMEM-01, was identified, which contained 66 genomically-dispersed array copies with diverse static barcodes (FIG. 1D, FIG. 6A and FIG. 6B). This cell line, which allowed recording in live cells with imaging-based readout of barcode base edits, static barcode sequences, and endogenous gene expression using multiple rounds of hybridization and imaging (FIG. 1E), was used for all subsequent experiments.

### Induction Drives Editing into Diverse Mutational States

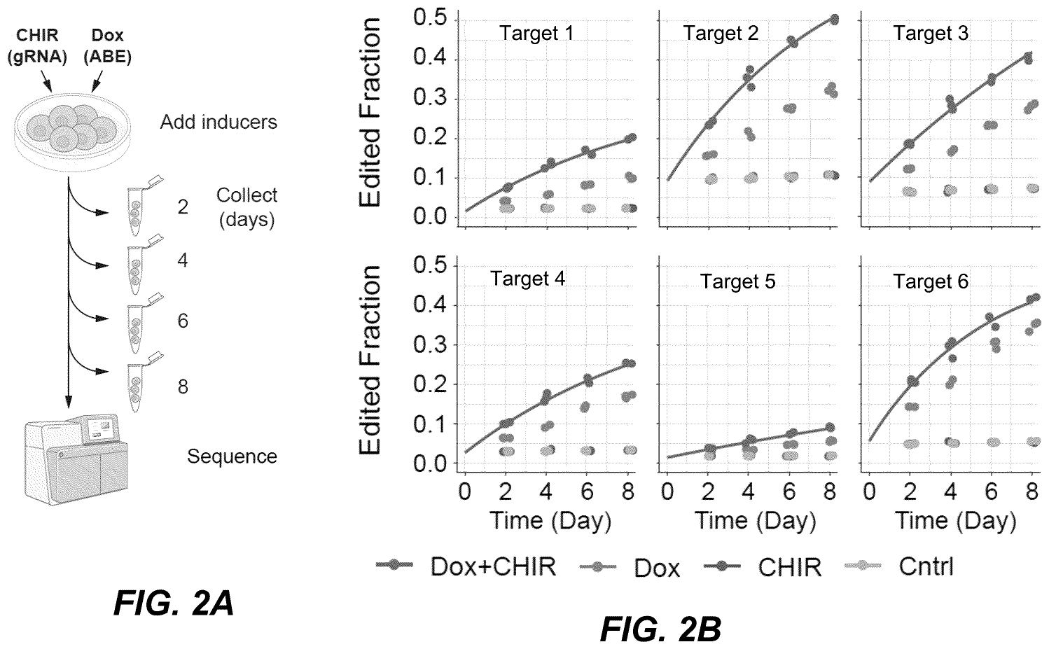

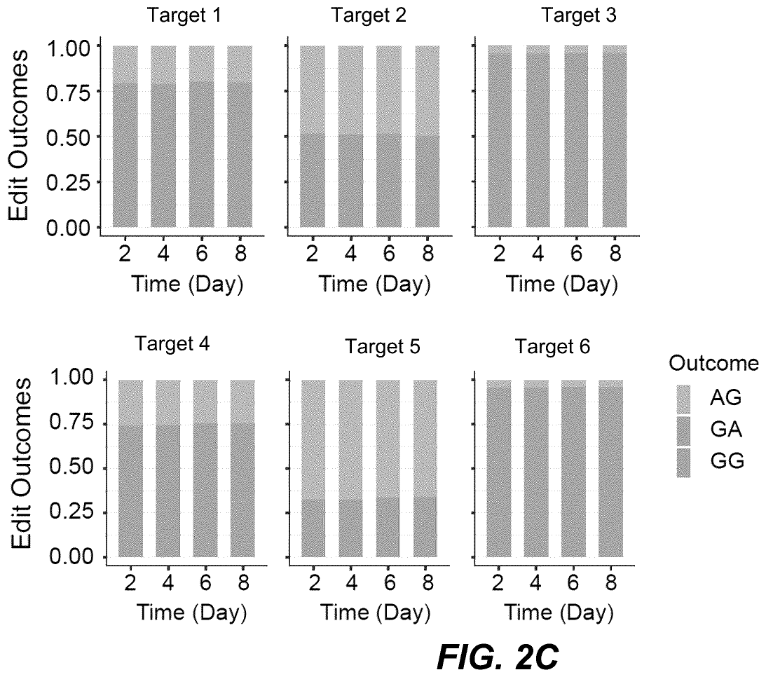

Since lineage recording depends on edits being accumulated on the timescale of cell division, inducible base editing using the system disclosed herein was analyzed. BaseMEM-01 mESC cultures were exposed to the inducers CHIR, doxycycline, or both, over an 8-day period, a timescale long enough to allow multiple stem cell generations and, in an embryonic context, approach gastrulation. samples were collected at multiple time-points (FIG. 2A). Then, the editable barcodes were analyzed by next generation sequencing.

As shown in FIG. 2B, edits accumulated at all 6 target sites. Without induction and in the absence of dox, some editing was observed. However, the fraction of such background edited sites generally remained constant during the time course, consistent with transient background editing during cell line construction, but minimal basal editing in stable clones. By contrast, in the presence of both CHIR and dox, edits accumulated rapidly at a rate that was well-fit by a model of editing with distinct edit rates at each site (FIG. 2B, solid lines). Interestingly, in the presence of dox alone, editing still occurred, albeit at an attenuated rate. This non-zero edit rate could be due to basal transcriptional activity of the WRE promoter or the activation of gRNA expression by low levels of endogenous Wnt signaling in the mESCs. However, these results showed that doxycycline, with or without CHIR, could provide tight control of edit rate, making the cell line sufficient for lineage recording.

Next, the distribution of edit outcomes at each site was analyzed. Different sites exhibited distinct ratios of edit outcomes (FIG. 2C). For example, site 6 exhibited a strong bias towards GA, and relatively little editing to AG. By contrast, sites 1 and 4 were more uniform in their outcomes. These differences in outcome bias across target sites were likely driven by differences in the sequences surrounding the dinucleotide for each target, which is known to impact CRISPR-Cas9 cleavage and base editing. Regardless of how edits were biased, however, all target sites exhibited constant biases over time, consistent with the notion that bias is an intrinsic feature of the sequence context (FIG. 2C). Most importantly, this consistency indicates that the GA and AG edit outcomes were stable for at least 8 days and did not become further edited to GG over the timescales of these experiments. These results thus validated the feasibility of this design of using inducible base editors to produce multiple distinct, individually stable states.

### Imaging Recovers Edited Barcode Sequences

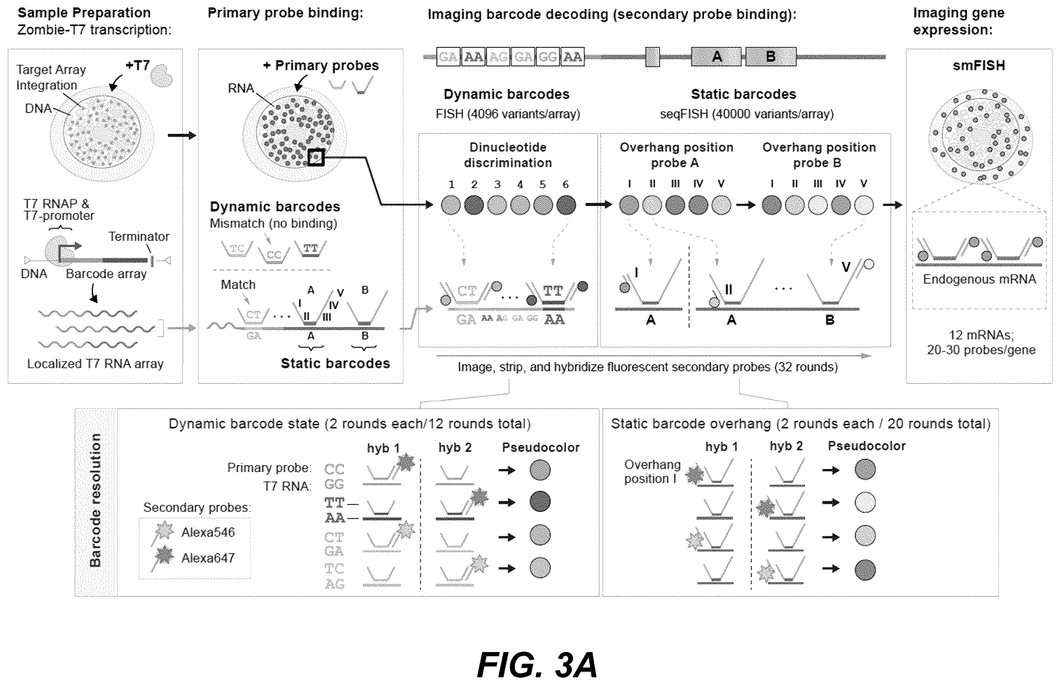

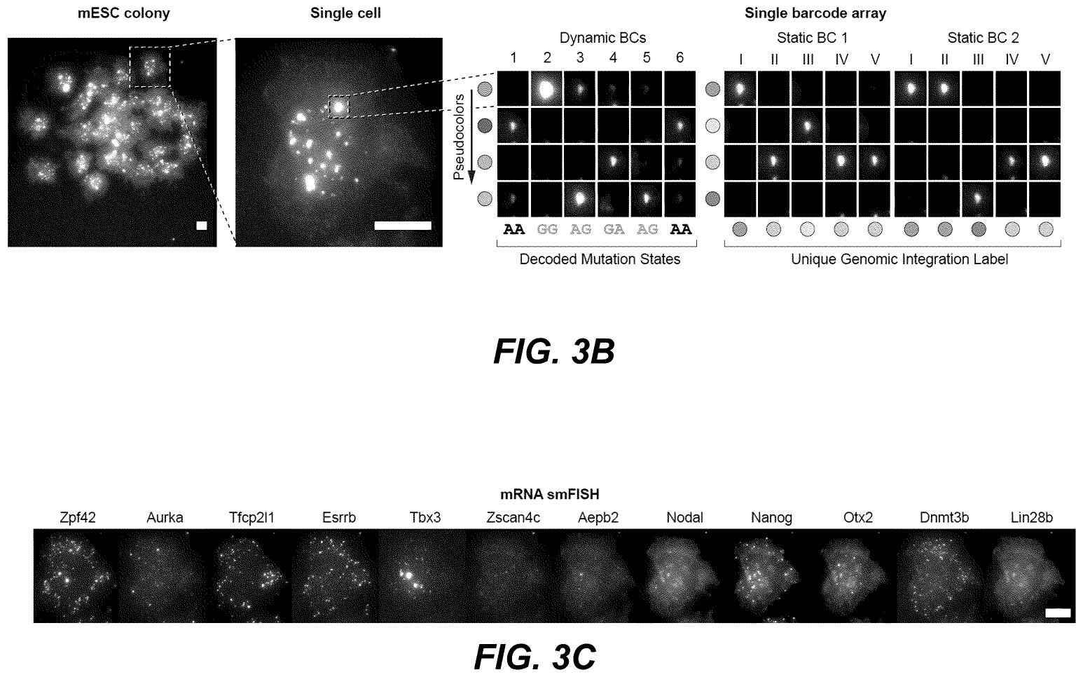

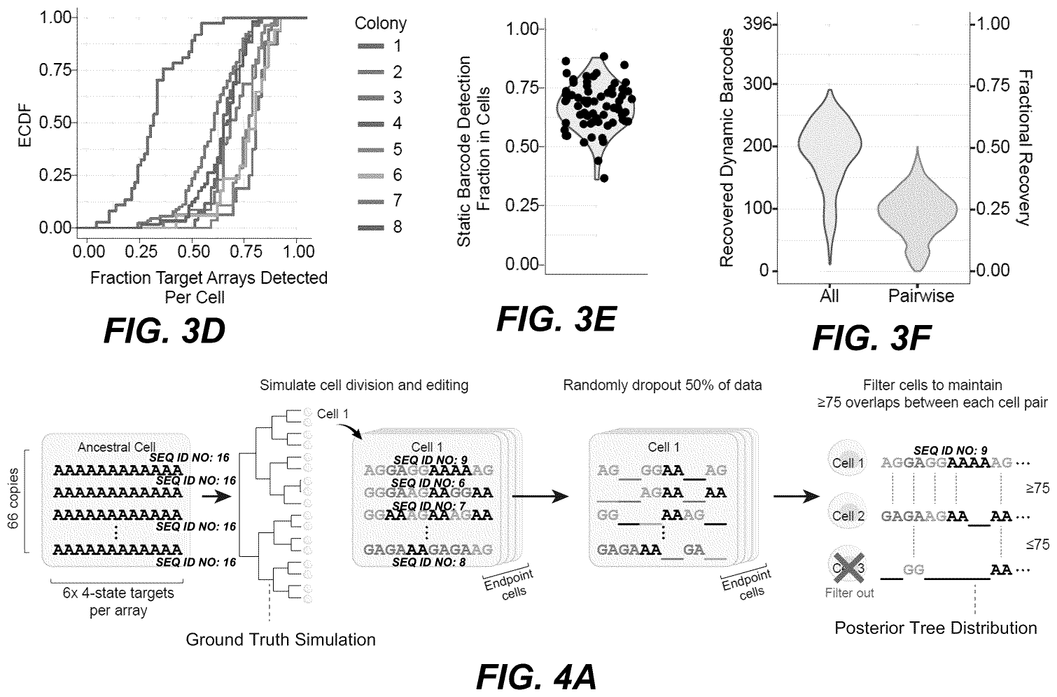

Next imaging readout was also evaluated. In situ barcode readout creates the opportunity to assay lineage relationships without disrupting spatial relationships among cells. An assay was developed to readout barcode sequences through multiple rounds of single molecule RNA FISH (smFISH). Specifically, baseMEM-01 cells were cultured for 3 days, a period long enough for several cell generations, under editing conditions (3 μM CHIR, 1 μg/mL dox). Cells were then fixed and in situ T7 transcription of barcodes was performed using the previously described “Zombie” approach (FIG. 3A, upper and lower left panels). Next, pools of 24 primary probes designed to bind to one of the 4 possible dinucleotide states in each of the 6 target sites (FIG. 3A and FIG. 3B) were hybridized. During hybridization, 3 primary probes were included for each of the 400 different 80 bp potential static barcode sequences, for a total of 1200 probes altogether (FIG. 1C, FIG. 3A and FIG. 3B). Additional primary probe sets were also included to analyze 12 different endogenous mRNAs, known to distinguish mESC pluripotency states in serum-LIF media (FIG. 3A and FIG. 3C).

After hybridizing all primary probes, sets of fluorescently-labeled secondary probes, designed to hybridize to corresponding “overhang” sequences engineered in the primary probes, were sequentially added (FIG. 3A, lower panels). For each set of secondary probes, cells were imaged in all channels, and secondary probes were then stripped to enable the next round of secondary hybridization and imaging. Two orthogonal fluorescent channels were used to halve the number of rounds of hybridization required. Membranes were also labeled with dye-conjugated wheat germ agglutinin to enable cell segmentation. All imaging was performed on a wide-field fluorescence microscope equipped with an automated fluid handling system. This procedure, similar to that used for seqFISH and related approaches, allowed systematic probing of dynamic barcodes, static barcodes, and endogenous genes over a total of 50 rounds of hybridization and imaging.

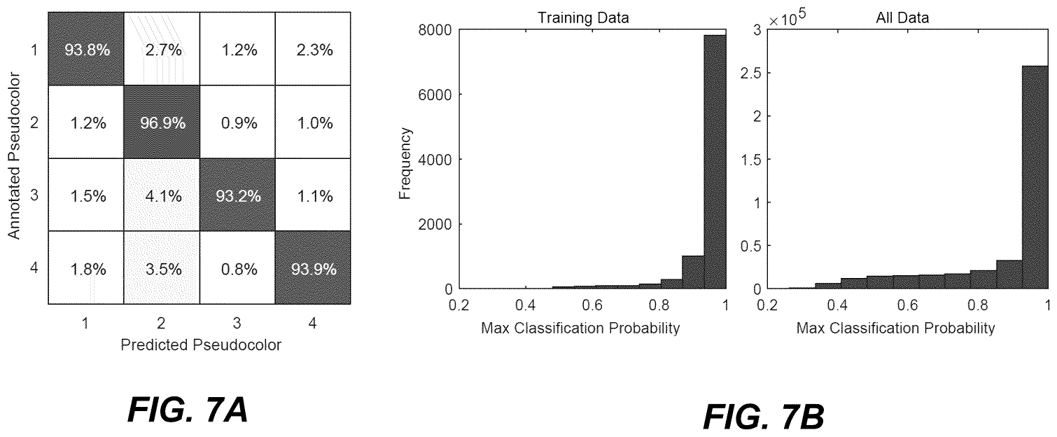

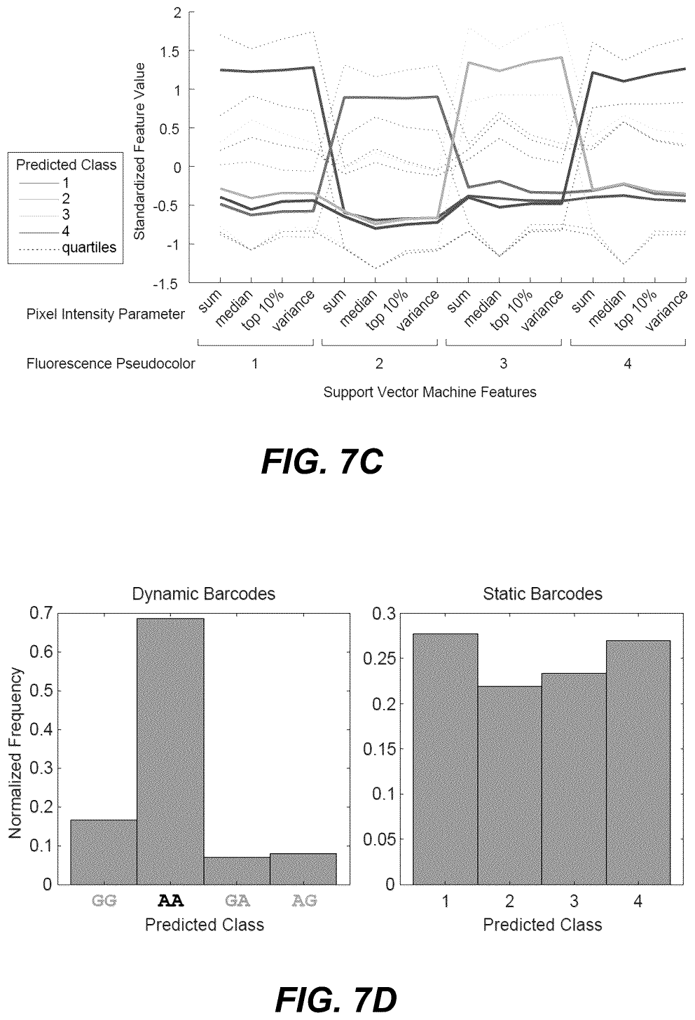

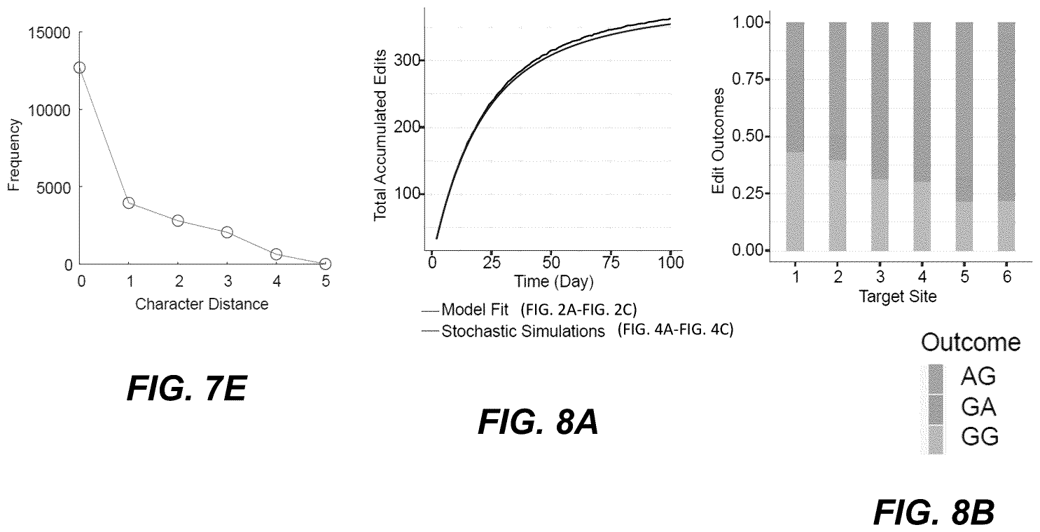

In the resulting image sets, individual target arrays could be identified as bright spots across multiple rounds of imaging. Most dynamic barcodes and static barcodes could be uniquely identified by a pseudocolor or set of pseudocolors (rows in image grids, FIG. 3B). A computational pipeline was developed to detect target array spots and classify the barcode states within each target array (FIG. 7A-FIG. 7E). Across 8 mESC colonies, roughly 50-80% of the 66 uniquely integrated target arrays were detected in any given cell (FIG. 3D). One colony had many fewer arrays detected than the others and was excluded from subsequent lineage analysis. The fractional detection of each unique barcode integration among all cells was broad but unimodally distributed, consistent with noisy detection efficiency in the absence of strong systematic differences among integration sites (FIG. 3E). Overall, after applying quality controls, roughly 200 high confidence dynamic characters, i.e. editable dinucleotides, per cell were detected (FIG. 3F). The most relevance detection statistics with lineage reconstruction was that ˜100 shared characters could be confidently recovered in both members of any cell pair.

### Simulations Show that baseMEMOIR can Accurately Reconstruct Detailed Lineage Trees

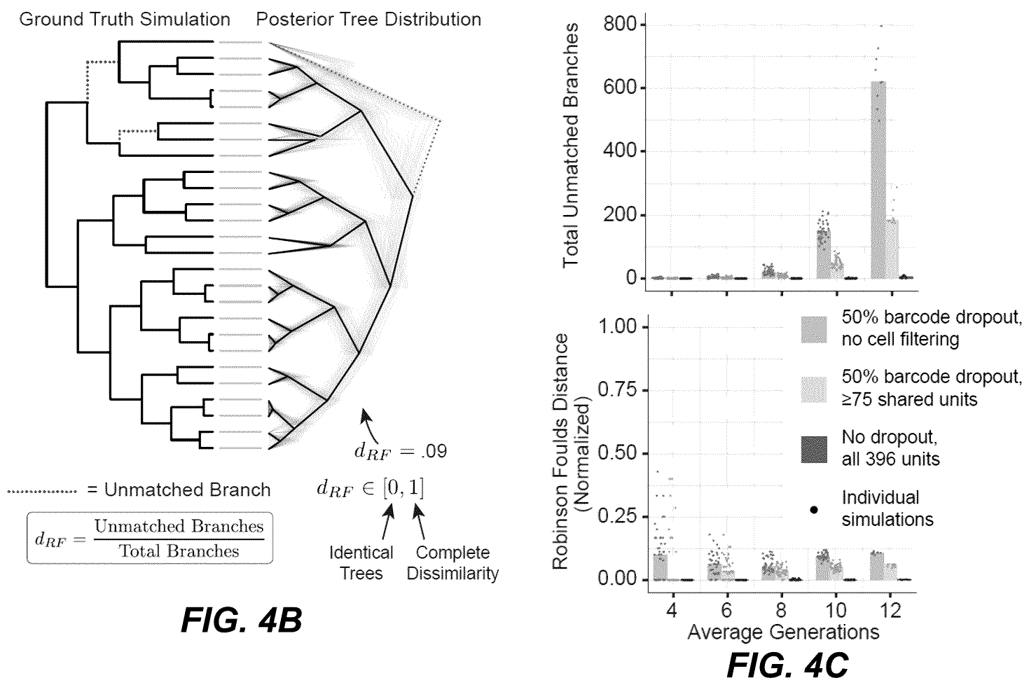

The dependency of the depth of tree reconstruction on the distribution of shared characters between cell pairs, as well as other parameters (e.g., the rate and uniformity of cell divisions, the rate of editing, and the duration of recording) was determined. To address these questions, barcode recording and recovery were simulated. During the recording phase, editing was simulated within a single initial cell and its progeny for up to 12 cell generations (FIG. 4A), setting barcode edit rates based on a time course editing dataset as shown in FIG. 2B and FIG. 2C. To simulate incomplete recovery of edit patterns, the resulting barcode sequences corresponding to the observed empirical recovery distribution were stochastically subsampled after hybridization and imaging (FIG. 3F). As a result, different cell pairs shared different fractions of recovered barcodes (FIG. 4A, middle right). Finally, a filtering strategy was explored to improve reconstruction accuracy by restricting analysis to cell pairs with a minimum number of shared recovered barcodes (FIG. 4A, right). This filtering strategy was at the cost of reduced numbers of cells per tree.

Next, lineage trees were reconstructed and compared to the ground truth trees from the forward simulations (FIG. 4B). As a metric of reconstruction accuracy, the normalized Robinson-Foulds distance was used to quantify the fraction of unmatched branches between the ground truth and reconstructed trees. For reconstruction, the Bayesian BEAST2 phylogenetic reconstruction framework was adapted, by incorporating a custom base editing model. BEAST2 used Markov Chain Monte Carlo (MCMC) sampling to estimate the posterior probability distribution over different tree topologies and other system parameters. Briefly, BEAST2 sampled a forest of possible trees in proportion to their probability density (FIG. 4B). As a Bayesian method, it allowed for model-based inference, explicitly incorporated prior knowledge, and quantified uncertainty in reconstruction.

In the ideal case of full recovery of all barcode edits in all cells, near perfect recovery of full lineage relationships for trees up to 12 cell divisions deep was obtained (FIG. 4C). When ˜50% of barcodes were lost, error rates for the same 12 generation tree increased to ˜10%. However, this error rate could be reduced by restricting analysis to cells sharing at least 75 jointly measured barcode positions (FIG. 4C). In contrast to the simulations, the experimental system could introduce additional factors, such as errors in barcode readout, variability in mean edit rates between cells, and pre-existing edits in the ancestral (root) cell. Nevertheless, these simulations suggested that baseMEMOIR, with empirically observed error and edit rates, should be capable of reconstructing multi-generation lineage trees with cell cycle resolution at ˜90% accuracy.

### BaseMEMOIR Reconstructs Lineage Trees in mESC Colonies

mESCs are known to undergo spontaneous reversible transitions among a set of molecularly and functionally distinct cell states, ranging from 2C-like to formative and primed epiblast-like states, in serum-leukemia inhibitory factor (LIF) conditions. A fundamental question about the mESC state-switching process is the structure of the transition graph, i.e. which transitions occur, at what rates, and how those rates are influenced by input signals. In particular, CHIR, a Wnt pathway agonist that is often used to maintain pluripotent cells, could impact the observed cell states and their transitions. Although CHIR was known to promote expression of key pluripotency genes and self-renewal in this system, the effects of CHIR on single cell state transition dynamics remained unknown.

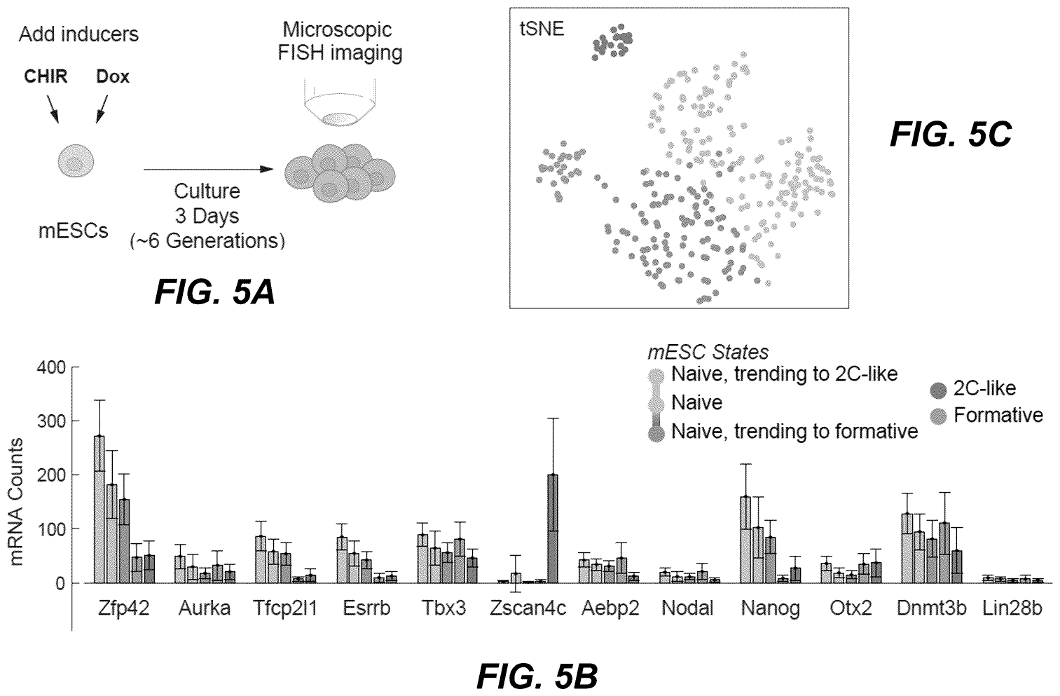

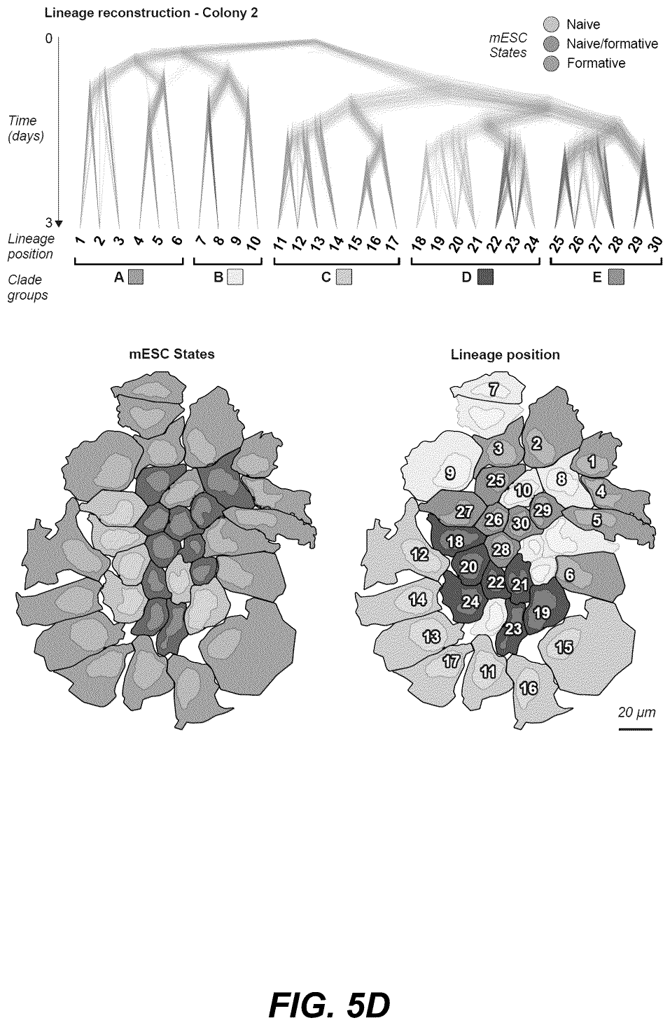

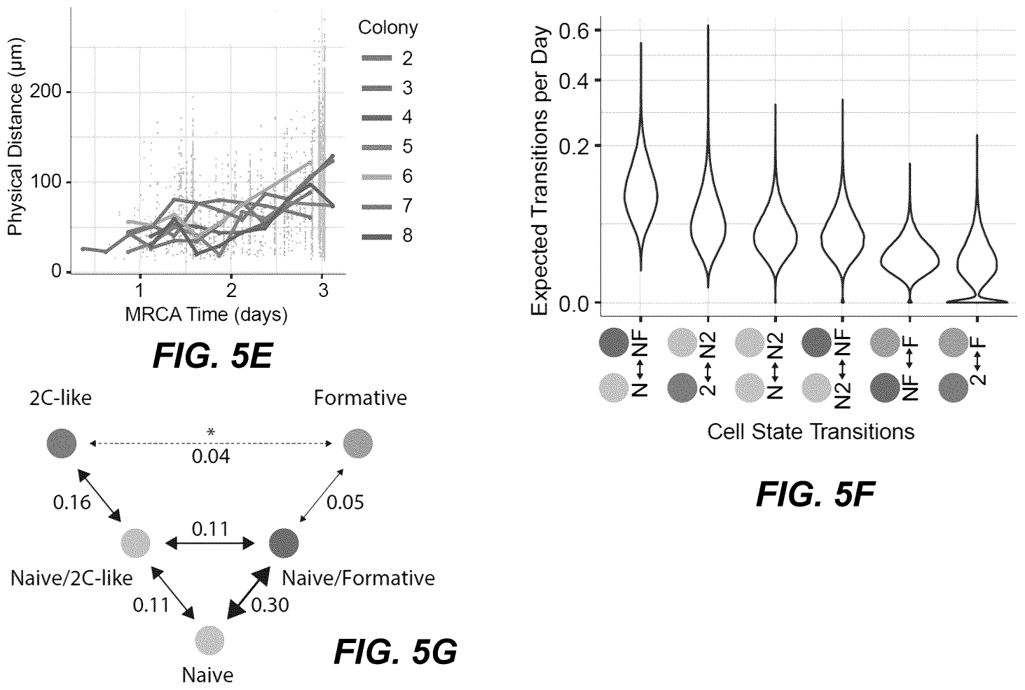

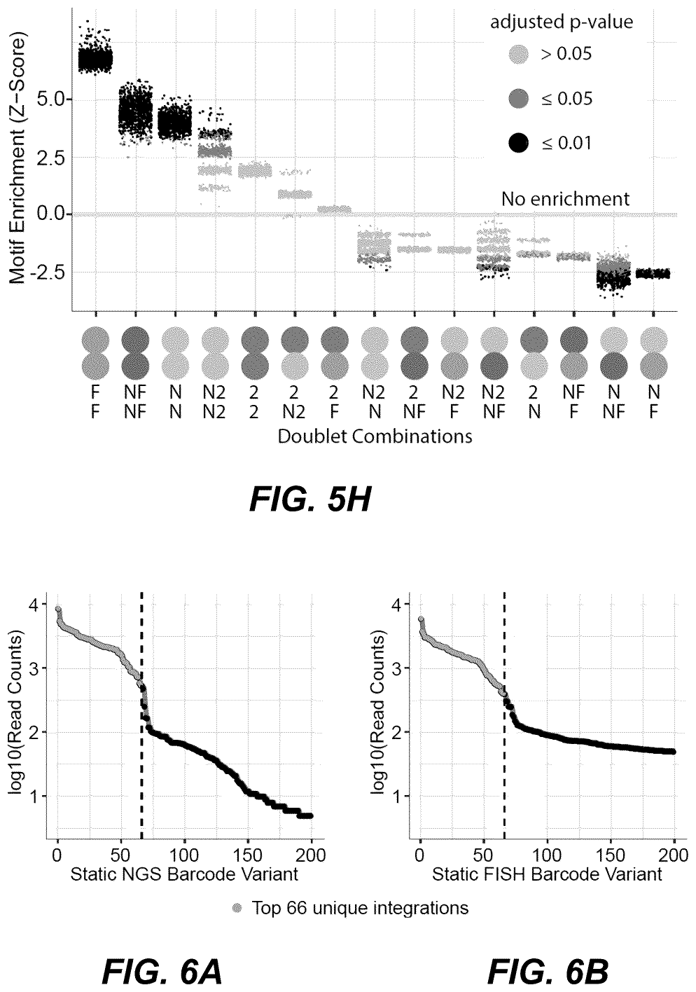

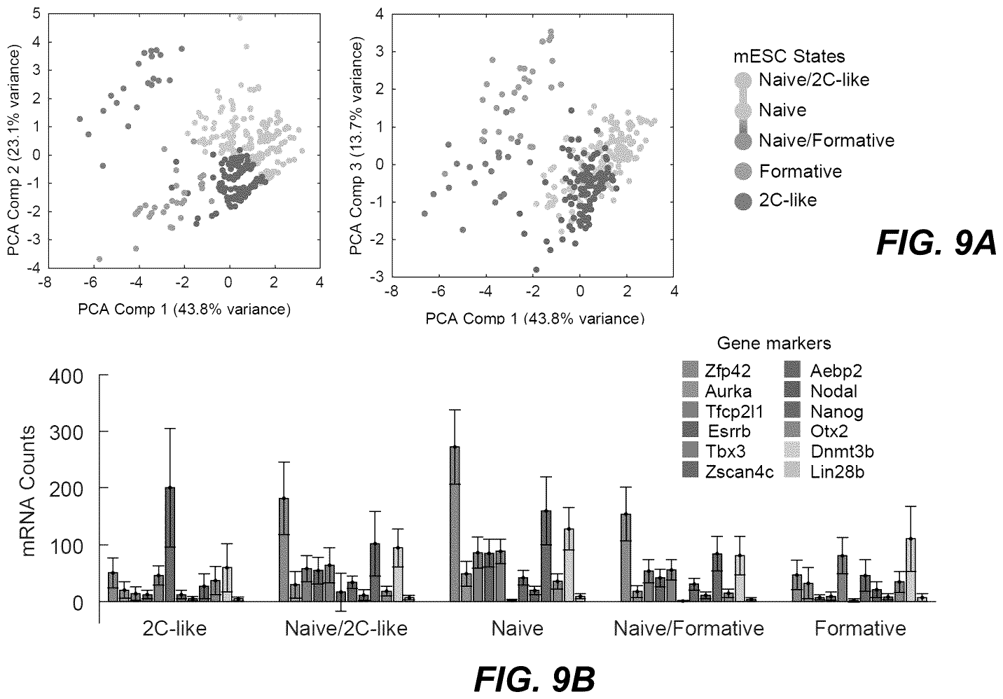

To study these dynamics, mESC colonies were grown over a 3-day period in the presence of CHIR and Dox, which also served to induce editing (FIG. 5A). After 3 days, the colonies were imaged as described above and barcode states for 7 colonies out of 8 total measured were recovered (FIG. 3D). In addition to reading out barcode states, gene expression levels for 12 pluripotency state markers was also recovered and, then, clustered to identify 5 gene expression states (FIG. 5B, FIG. 5C, FIG. 9A and FIG. 9B). Three major cell states were identified. These major cell states comprises: 2C-like cells with high expression of Zscan4C; naive cells expressing transcription factors Nanog, Esrrb, and Zfp42; and formative cells that express Otx2 and Dnmt3b in the absence of naive pluripotency factors and Zscan4C (FIG. 5B). Naive cells exhibited a distribution of gene expression levels that varied from more 2C-like to more formative (FIG. 5B, FIG. 5C, FIG. 9A and FIG. 9B). These states largely corresponded to those described in previous work, although high and relatively homogeneous Tbx3 expression across all cell states was observed, which was in contrast to observations of cells grown in serum/LIF without CHIR. This difference was consistent with previous work showing that Tbx3 was significantly regulated by CHIR in the observed direction.

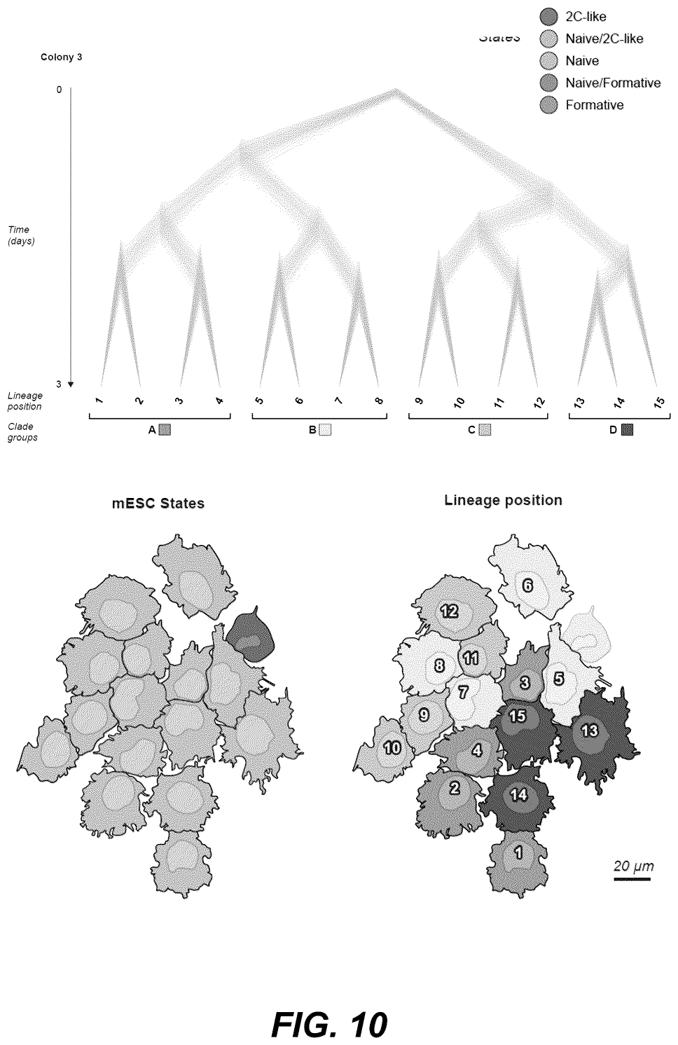

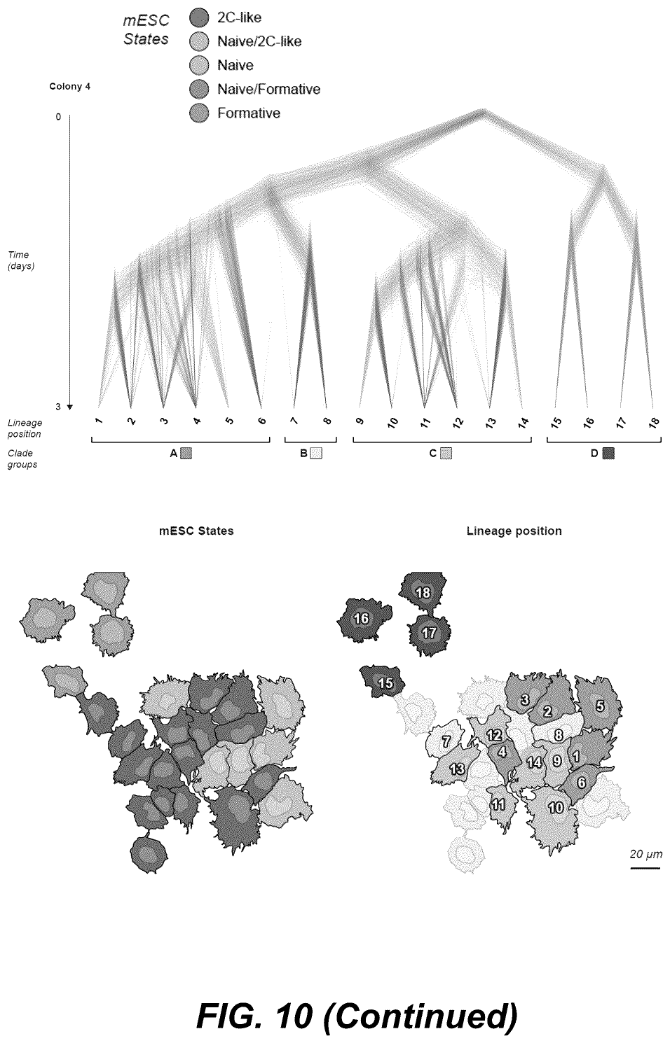

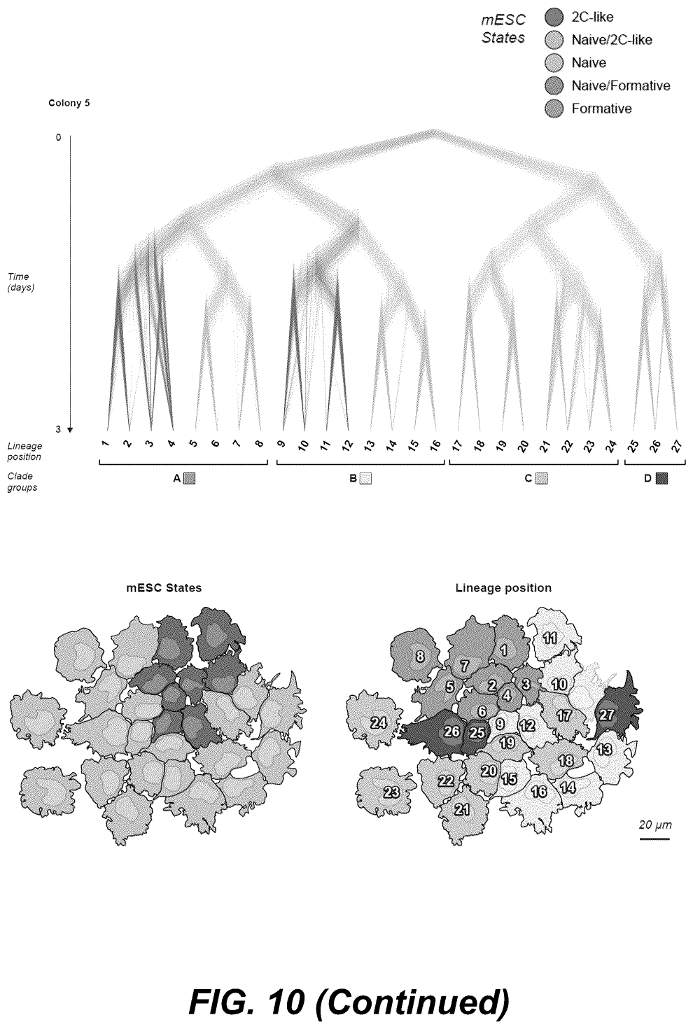

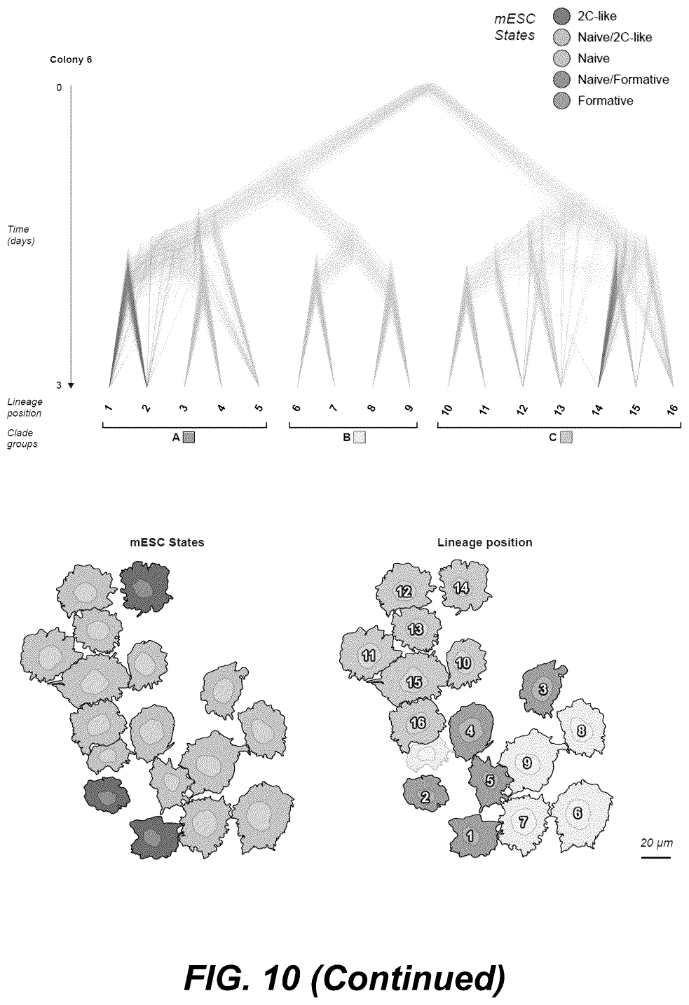

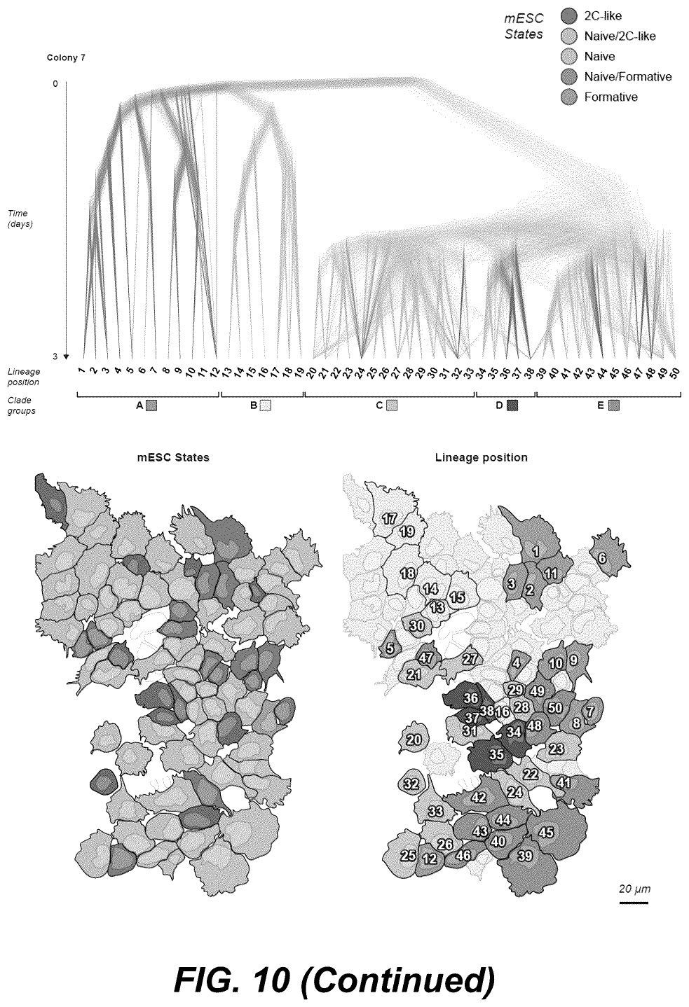

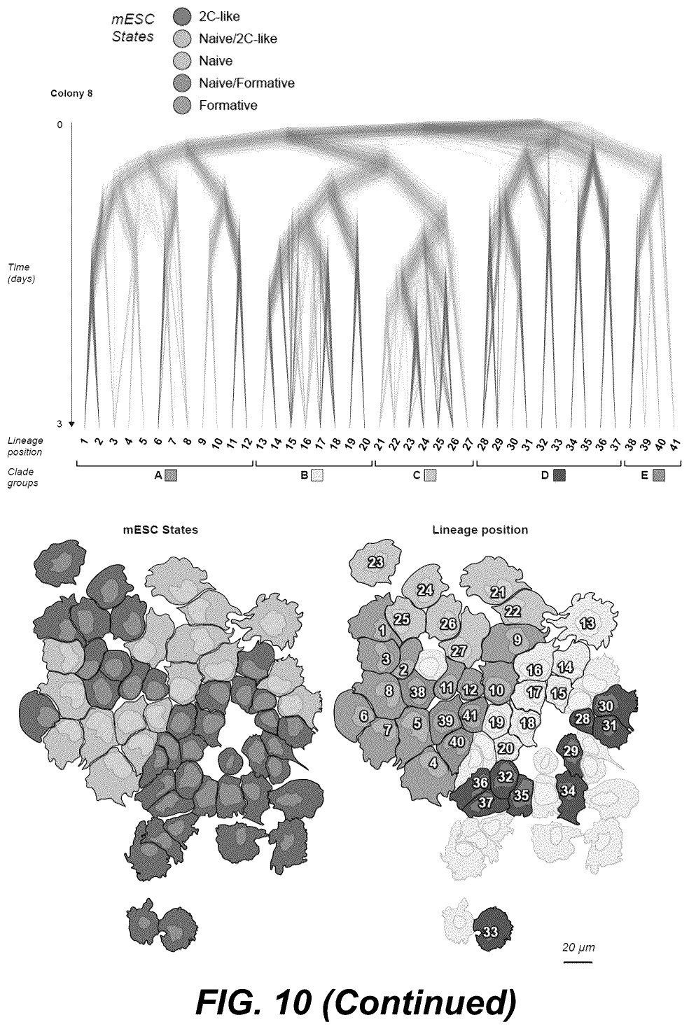

The BEAST2 system described above was applied to reconstruct lineage trees for cells in these colonies. To incorporate information of cell state and spatial position, the underlying editing model (FIG. 4A-FIG. 4C) was extended to represent these additional cellular properties. The application of this model allowed simultaneous inference of cell state transitions and spatial movement alongside cell lineage. Applied to the mESC colonies, this approach reconstructed lineage relationships and division times with relatively low uncertainty in most cases, as indicated by the limited “fuzziness” of the reconstructed trees (FIG. 5D and FIG. 10). However, there were some ambiguities in reconstruction. For example, in FIG. 5D, cell 23 is roughly equally likely to be a sister of cell 22 or cell 24. This illustrated the way in which uncertainties in the Bayesian reconstruction still provided specific alternative hypotheses rather than numerical confidence values. Additionally, in some cases, not all neighboring cells were captured. Capturing all neighboring cells can introduce branch lengths longer than a single cell division, as shown, for example, in clades A and B of colony 7 in FIG. 10. Together, these results demonstrated that the recording system allowed precise lineage reconstruction of 3-day clonal mouse ESC colonies, with tree sizes of 30-50 cells.

The reconstructions also allowed estimation of cell state transition dynamics. To constrain the transition model, transitions were treated as a reversible, symmetric, continuous time Markov process. Of the 10 possible symmetric interactions, posterior estimates suggested that ˜5 occurred at appreciable rates during the growth of these colonies (FIG. 5F and FIG. 5G). A sixth transition, between 2C-like and formative states, was suggested by a single event in colony 7 (FIG. 10). This event was unexpected given previous inference of chainlike dynamics, with these two states at opposite ends. However, it could be a result of CHIR exposure, which was not present in previous work. Furthermore, a substantial amount of posterior probability indicated a negligible rate for this transition (FIG. 5F and FIG. 10). However, more data would be needed to clarify this result. Overall, these reconstructions suggested frequent (e.g., median of 0.15 transitions per day across all colonies) transitions among Naive/2C-like, Naive, and Naive/Formative states, and frequent conversion between Naive/2C-like and 2C-like states (FIG. 10). Interestingly, the inferred transitions correlated with expectations based on transcriptional similarity among states, even though this information was not provided to the model (FIG. 5B, FIG. 5C, FIG. 9A and FIG. 9B).

### BaseMEMOIR recovers lineage relationships, cell states, and spatial relationships in mESC colonies

By mapping lineage trees back onto the original images, spatial and lineage organization of colonies were simultaneously observed (FIG. 5D, lower panels). This analysis revealed correlations between lineage history, spatial position, and cell fate. For example, in FIG. 5D, the related D and E clades contained Naive and Naive/Formative cells, and were located towards the interior of the colony, while cells in clades A, B and C were largely in the Formative state and located around the periphery. Individual cell morphologies also varied systematically, with cells in the periphery exhibiting larger sizes. Other colonies were less radially structured, but still showed strong correlations between spatial positions and lineage relationships within each colony (FIG. 5E and FIG. 10). These results showed that it was possible to impose lineage relationships on spatial colonies with cell state information.

Lineage motifs provided a complementary approach to analyzing cell state transitions. They were defined as statistically over-represented patterns of cell fates on lineage trees, which reflected features of underlying stochastic cell fate control programs. As an example, asymmetric division, in which sister cells acquired opposite fates, would be reflected in the enrichment of opposite fates among sibling pairs, which were named “doublets.” In contrast to the inference of transition rates described above, lineage motifs can be identified with no assumptions about an underlying model. Applying Lineage Motif Analysis (LMA) to 1000 samples from the Bayesian posterior tree distribution, 4 overrepresented doublet pairs were identified with an adjusted p-value less than 0.05 for a majority of the posterior samples (FIG. 5H). These cases involved siblings in the same state, consistent with infrequent state transitions. Siblings in the formative state were the most overrepresented, mirroring results from the Bayesian Markov model, which predicted the slowest transitions to and from the formative state (FIG. 5F and FIG. 5H).

Two statistically underrepresented heterogeneous sibling pairs were also observed (FIG. 5H). The most underrepresented pair, containing naive and formative cells, was also qualitatively consistent with predictions of the Bayesian Markov model, which identified a negligible transition rate between these states. Additionally, the naive and naive/formative sibling pair was also significantly underrepresented. This corresponded to the most rapid inferred transition rate in the dataset (FIG. 5F), consistent with high rates of independent transitions out of either the naive or formative states. Together, these results demonstrated how baseMEMOIR's lineage reconstruction ability allowed inference of lineage motifs.

Finally, the lineage reconstruction was combined with spatial and cell state dynamics to infer a property (e.g., the relative spatial mobilities of different cell states) that would be difficult to analyze from sequencing-based readout or static snapshots alone. The inferred histories of cell state and spatial position can be visualized as movies (See Supplemental Materials incorporated by reference above). These movies can represent one possible history based on a simple model of cell diffusion, taking the highest credibility inference from BEAST2. Together, these results showed how spatial position, cell state, and lineage were analyzed and reconstructed together, and used to infer features of cell histories.

### Discussion

In the field of biology, imaging a tissue or organism and visualizing not only its current state, but also its past history have been desired for a long time. Previous work has attempted to meet this need in different ways, including lineage recording by accumulation of irreversible recombination events and reconstruction of small trees. However, these efforts were limited in the amount and scalability of memory storage. A new approach, baseMEMOIR, is herein disclosed, which provided much larger memory sizes and allowed for deeper, more accurate lineage tree reconstruction, while preserving spatial structure.

To achieve this, baseMEMOIR introduced several key innovations. First, it used base editors to introduce stochastic, but precise, edits at dense target arrays (FIG. 1C). Second, it used dinucleotide editable target sites, each of which were edited to any of three permanent end states (FIG. 1B and FIG. 2C). Third, to distinguish among those states, the Zombie readout system was expanded to allow 4-way probe competition (FIG. 3A and FIG. 3B). Fourth, baseMEMOIR massively expanded the amount of memory accessible in single cells by incorporating 66 unique statically barcoded target arrays, collectively providing 792 bits of editable information in the baseMEM-01 cell line. This number can be readily increased with additional target array integrations without modifying other components of the system. Fifth, baseMEMOIR achieved high density recording, while maintaining compatibility with FISH-based readout of endogenous genes (FIG. 3C). Finally, to address the challenge of lineage reconstruction from stochastic edits, the BEAST2 framework for Bayesian tree inference was adapted, both by adding a new mutation model and taking advantage of its phylogeographical and discrete trait models. This probabilistic framework should be applicable for a broad variety of lineage recording methods.

To demonstrate these capabilities, baseMEMOIR was applied to stem cells undergoing interconversion among transcriptional states. This allowed us to reconstruct lineage trees for 7 colonies totaling 197 cells, with as many as 4-7 cell generations per colony (FIG. 5D and FIG. 10). Furthermore, transition rates for specific pairs of states were able to be inferred. These rates were consistent with a role for Wnt, activated through CHIR, in influencing state dynamics relative to similar cultures in the absence of Wnt (FIG. 5F and FIG. 5G). In some embodiments, baseMEMOIR is employed to systematically compare the effects of different signals and perturbations on cell state dynamics. While probabilistic inference was not an equivalent to direct time-lapse observation, it nevertheless yielded related insights that would ordinarily be concealed from any static endpoint measurements. In some implementations of this system, such as those containing either more memory or linking signaling pathway activity to recording machinery, it is possible to infer increasingly detailed views of earlier dynamic events in complex multicellular settings, effectively. Such detailed view can add events (e.g., changes in cell state or even movements in space) to the lineage trees. BaseMEMOIR should also allow the inference of state-switching dynamics and developmental programs using approaches such as Kin Correlation Analysis and Lineage Motif Analysis that exploited lineage tree information.

In some embodiments, the employment of baseMEMOIR did not directly probe the states of cells at earlier time points, so it cannot directly detect earlier states that do not appear in the endpoint measurement, and thus analyzing systems at multiple timepoints can help to avoid missing transient states. Additionally, in some embodiments, (i) cells that died or migrated away prior to measurement can be omitted from the tree and could confound estimates of variation in cell cycle durations in different lineages, and/or (ii) there can be a failure to recover sufficient barcodes from an individual cell could make it difficult to classify—and this can be addressed in some embodiments by barcode imaging adjustments.

BaseMEM-01 can immediately be used to explore stem cell differentiation and early mouse embryogenesis, among other phenomena. BaseMEMOIR can be readily adaptable to diverse developmental and physiological processes. The constructs and system can be transplanted to additional cell types using standard methods, and potentially combined with readout of additional “multi-omics” information such as chromatin accessibility. therefore, augmenting spatial cell atlases with lineage information1, and using baseMEMOIR to investigate the role of lineage, signaling, and differentiation in disease progression can be anticipated.

### Methods

### Dynamic Barcoding Strategy

Dynamic barcode sequences consisted of 20 bp CRISPR target sites with 3 bp downstream NGG PAM sequences. These were chosen by designing sequences with AA nucleotides at the location predicted to be edited by the ABE (e.g., positions 5-6 in the protospacer sequence), then screening them for significant, varied editing of the AA sites. Six unique target sites were arrayed sequentially downstream of a T7 promoter sequence to enable imaging-based readout as described below (FIG. 1C).

### Static Barcoding Strategy

Static barcodes consisted of two variable 80 bp sequences downstream of the 6 dynamic barcode targets (FIG. 1C). A pooled plasmid library was formed by generating constructs with 200 variants at each of the two 80 bp regions, for a total of 40,000 unique sequences (See Supplemental Data of Chadly et al. incorporated by reference above). Each sequence contained three unique primary probe binding sites for signal amplification during FISH readout (See Supplemental Data of Chadly et al. incorporated by reference above).

### Plasmid Construction

Plasmids were constructed in piggyBac backbones for later transposase mediated integration into the genome. The inducible ABE plasmid was made by integrating a tet-responsive promoter (TRE3G, Takara Bio) and ABE 7.10 (Addgene #102919) into a piggyBac plasmid with neomycin resistance. The Tet-On 3G protein gene used to activate the ABE in a doxycycline dependent fashion was supplied as a piggyBac plasmid with a pEF promoter and puromycin resistance.

The dynamic and static barcode arrays were constructed in a piggyBac vector containing hygromycin resistance and double T7-T3 promoter sites followed by the dynamic barcode array, which was synthesized by Integrated DNA Technologies (IDT). The static barcode was then integrated 3′ of the dynamic barcode array. The static barcode was composed of two sites of 80 bp each, with 200 possible sequences for each of the two sites to give an overall possible barcode diversity of up to 40,000 unique sequences. The static barcode sequences were synthesized by Twist Bioscience and amplified with the appropriate cloning ends by PCR. The 5′ primer for the first static barcode site had a set of 10 random nucleotides to provide a further NGS-readable ID to each barcode. A mix of Gibson and sticky end cloning were used for plasmid construction.

The plasmid library containing static barcodes was generated by transforming high-efficiency competent cells (e.g., NEB C3019), then plating them onto a large surface area of LB-agar (˜30 10-cm petri dishes) to generate a large number of colonies. These were scraped and pooled into a single liquid culture. Subsequently, plasmid DNA was collected using multiple DNA Miniprep columns (Qiagen 27104) and pooled.

An array of six gRNAs targeting the six sites of the dynamic barcode were integrated in the 3′ UTR of an NLS-mTurquoise gene. Each gRNA sequence was flanked by the hammerhead and HDV ribozyme sequences on upstream and downstream sides, respectively, in order to excise the gRNA from the transcript. These gRNA-ribozyme sequences were each synthesized as gBlocks by IDT and combined by assembly of unique sticky end junctions into the piggyBac plasmid. A Wnt-responsive promoter was integrated to drive expression of the m Turquoise-gRNAs construct. This plasmid included blasticidin resistance for subsequent mammalian selection.

### Primary Probe Library Construction

Primary probes for dynamic barcode readout were purchased from IDT as individual sequences. The primary probe library, containing 1200 probes targeting all static barcode variants across both regions, including 3 probes per variant, 200 variants per region and 2 regions, was ordered as an oligo-array pool from Twist Bioscience. Each probe was assembled with a 35-nucleotide sequence complementary to the static barcode sequence, five 15-nucleotide readout sequences uniquely labeling each variant separated by a 2-nucleotide spacer, and two flanking primer sequences to allow for PCR amplification of the probe library. An exemplary structure can be: 5′-(primer 1)-(readout 1)-(readout 2)-(probe)-(readout 3)-(readout 4)-(readout 5)-(primer 2)-3′. The probe library was amplified following an established protocol described in a previous report.

Endogenous marker genes were selected based on previous work. Probes for non-barcoded sequential smFISH of gene markers were generated, using a single readout sequence repeated four times in place of a unique barcode (structure 5′-(primer 1)-(readout 1)-(readout 1)-(probe)-(readout 1)-(readout 1)-(primer 2)-3′).

### Readout Probe Synthesis

Fluorescently conjugated secondary readout probes 15-nt in length were designed as in previous work. Probe sequences were ordered conjugated to AlexaFluor 546 or 647 from IDT as indicated (See Supplemental Data of Chadly et al. incorporated by reference above).

### Coverslip functionalization

24×60 mm coverslips were functionalized prior to cell culture. Coverslips were first rinsed in 100% ethanol, then dried and functionalized using a plasma cleaner on the high setting for 5 minutes. Coverslips were subsequently immersed in 1% bind-silane (GE, 17-1330-13) solution (1% bind silane and 10 mM acetic acid in 90% ethanol) for 1 hr at room temperature. Coverslips were rinsed in 100% ethanol and heat dried in an oven at 90° C. for 30 minutes before being treated with 100 μg/mL Poly-D-Lysine in water overnight. On the next day, slides were rinsed with nuclease free water and air dried. Slides were stored for up to 2 weeks at 4° C. prior to use.

Just before cell attachment, coverslips were treated with UV in a biosafety cabinet for 5 minutes. Then, the surface was treated with 10 μg/mL laminin (Biolaminin 511 LN, Biolamina) at 37° C. for 90 minutes. Laminin was removed, then cell suspension was added directly to the surface for attachment.

### Cell Culture

E14 mES cells (ATCC cat. No. CRL-1821) were cultured in medium containing GMEM (Sigma), 15% ES cell qualified FBS (Gibco), 1×MEM non-essential amino acids (Thermo Fisher Scientific), 1 mM sodium pyruvate (Thermo Fisher Scientific), 100 μM B-mercaptoethanol (Thermo Fisher Scientific), 1×penicillin-streptomycin-L-glutamine (Thermo Fisher Scientific) and 1000 U/mL leukemia inhibitory factor (Millipore). For cell engineering and standard culture, cells were maintained on polystyrene (Falcon) plates coated with 0.1% gelatin (Sigma) at 37° C. and 5% CO₂.

### Cell Line Engineering

Sequences of all integrated constructs are reported as Supplementary Data of the Chadly et al. reference incorporated by reference above (and can be retrieved at biorxiv.org/content/10.1101/2024.01.03.573434v1.supplementary-material). BaseMEMOIR components were integrated over several rounds of transfection and selection. For all transfection steps, mESCs were cultured in 24 well plates, then co-transfected with the plasmid(s) to be integrated as well as piggyBac transposase plasmid with HD FuGENE transfection reagent. First, cells were co-transfected with ABE and Tet3G activator plasmids. The cells were allowed to recover for a day, passaged, and then underwent selection with 400 μg/mL neomycin followed by 500 μg/mL geneticin. Cells were plated sparsely in a 10 cm dish to grow monoclonal colonies. Then, the mono-clones were selected and grown in 96-well plates. Clones were screened for ABE expression after dox induction by qPCR, then subsequently by FISH to identify clones with homogenous expression among single cells.

Barcode target plasmids were integrated into the parental line containing inducible ABE by a second round of transfection, then selected with 100 ng/ml hygromycin. Monoclonal colonies were selected, then screened by qPCR for high relative copy number. Zombie-FISH was used to screen promising candidates and select the clone with the highest visible integration number.

Finally, gRNAs and additional ABE plasmid were integrated into the most promising line from the previous step. Cells were selected with 15 ng/ml blasticidin. Then, monoclonal lines were generated as described above. Clones with a clear mTurquoise expression upon addition of 3 μM CHIR, which indicated expression of the gRNA construct, were kept for further analysis.

The best clones were tested for array targeting by adding 1 μg/mL doxycycline and 3 μM CHIR for multiple days followed by Sanger sequencing. Editing resulted in mixed peaks at the edited bases. One of the clones, baseMEM-01, was identified to have the most editing via this approach and was used for all subsequent experiments.

### Next Generation Sequencing

Genomic DNA was extracted from cells using the DNeasy Blood and Tissue Kit (Qiagen) according to manufacturer instructions. Amplicon libraries containing the dynamic barcode sequences and short NGS static barcodes were generated with a two-step PCR protocol to add Illumina adapters and Nextera i5 and i7 combinatorial indices. Indexed amplicons were pooled and sequenced on the Illumina MiSeq platform with a 600-cycle, v3 reagent kit (Illumina, MS-102-3003). Raw FASTQ files were aligned to a FASTA-format reference file containing the expected amplicon sequences. Alignment was performed using the Burrows-Wheeler alignment tool (bwa-mem). Subsequent analysis and data visualization was performed in the R statistical computing platform, v 4.1.1 (See Supplemental Data of Chadly et al. incorporated by reference above).

### Edit Accumulation Model

Edit accumulation at each target site was modeled by fitting Equation (1):

$$E = \frac{p}{ln ( 1 - p )} [ {( 1 - p )}^{t + d} - 1 ] \qquad ( 1 )$$

In Equation (1) above, edit accumulation (E) is a function of time (t) with parameters including the probability of editing per unit time (p) and the duration of time (d), during which edits accumulated prior to the zero time point. The edits accumulated prior to the zero time point accounted for empirically observed background edits as shown in FIG. 2B. This relation can be derived by assuming a probability (p) of a target site being editing per unit time (t) in a long string of target sites. After a unit of time t, p edited targets and (1−p) unedited targets were expected. By the same logic, after another time step, (p+p(1−p) edited targets and (1−p)² unedited targets were expected. After T time steps, the expected edited targets were:

$$p {\sum }_{t = 0}^{T - 1} {( 1 - p )}^{t} \qquad ( 2 )$$

Taking the limit of a discrete time step dt approaching zero, this sum can be approximated by the integral

$$p {\int }_{0}^{T} {( 1 - p )}^{t} dt \qquad ( 3 )$$

which was simplified to Equation (1).

Parameters were fit to editing time course data (FIG. 2A-FIG. 2C) to determine the empirical edit accumulation rate for each target using the “nls” function from the “stats” package in R (See Supplemental Data of Chadly et al. incorporated by reference above).

### Stochastic Simulations

Barcode editing was simulated in R using the Gillespie method (See Supplemental Data of Chadly et al. incorporated by reference above). Separate propensities were estimated for each editing outcome and target site by multiplying the edit accumulation parameter p in Equation (1) for each target site by the observed mean outcome proportion at each target site across time (FIG. 2C). This stochastic simulation method recapitulated both the edit accumulation model fit and the empirical target state outcome distribution (FIG. 8A-FIG. 8B).

Cell division was modeled by allowing editing until a predetermined cell division time, after which barcodes were duplicated before allowing editing to continue. Cell division waiting times were drawn from a distribution derived from Eyring-Stover survival theory that had been shown to model cell division times more accurately than the exponential distribution.

Lineage relationships were reconstructed based on the resulting barcodes using BEAST2 software as described below, considering only barcode data. Reconstructed trees were compared to simulated ground truth trees by computing the normalized Robinson-Foulds distance as implemented in the “RF.dist” function of the R package “phangorn.”

### Zombie Preparation

For Zombie and subsequent RNA-FISH, cells were plated on coverslips treated as described above. After culture and editing, coverslips were washed with 1 mL PBS with calcium and magnesium (PBS +/+) then fixed with a 1:1 solution of Methanol:Acetic Acid (MAA) for 20 minutes. RNase-free reagents were used for all subsequent steps to minimize RNA degradation. MAA was removed. Then, coverslips were transferred to a 100 mm petri dish and covered with 70% ethanol. Petri dishes were parafilmed and stored at −20° C. before imaging.

Immediately prior to imaging, coverslips were removed from cold storage and brought to room temperature. 70% ethanol was removed and replaced with a fresh solution of MAA. Then, the coverslips were incubated for 2 hrs at room temperature. The sample was washed twice with PBS +/+, incubating for 2-3 minutes between each wash. The final wash solution was removed. The sample was dried until all liquid had evaporated. A custom fluidic cell, built to interface with a custom designed liquid handling system, was affixed to the coverslip surface. Subsequent washes took place within the flow cell, by manually adding reagents into the inlet of the cell and then removing the reagents from the outlet using a standard micropipette. The cells were washed with nuclease free water once, which was then replaced with T7 RNAP mix (New England Biolabs E2040S). The sample was incubated at 37° C. overnight in a humidified tupperware.

The following morning, the T7 RNAP mix was removed and replaced with fresh T7 RNAP mix. Then, the sample was incubated for 1 hr at 37° C. in the humidified tupperware. The mix was removed, then the sample was immediately fixed with 4% paraformaldehyde for 10 minutes. The fixing solution was removed. The sample was washed three times with PBS +/+, and then washed with 30% formamide probe wash buffer (30% formamide in 5×SSC with 9 mM citric acid, 0.1% Tween-20, and 50 μg/mL heparin, pH 6.0) for an additional 5 minutes. The wash buffer was replaced with primary probe hybridization mix. Then, the sample was incubated overnight at 37° C.

### FISH Imaging

Images were collected across multiple rounds of fluorescence hybridization to identify barcode and cell states. Formamide wash buffers and secondary probe hybridization mixes were made immediately prior to imaging. A custom-built, automated liquid handling system was used to perform sequential rounds of in situ hybridization. Briefly, the sample was connected to an automated fluidics system attached to a widefield fluorescence Nikon Eclipse Ti microscope. The custom-made automated fluid sampler was used to transfer readout probes in hybridization buffer from a 2.0 mL 96 well plate through a fluidic valve (IDEX Health & Science EZ1213-820-4) to the custom-made flow cell using a syringe pump (Hamilton Company 63133-01). Fluidics and imaging were integrated using a custom script controlling uManager. Eleven fields of view (FOVs), capturing 8 well separated regions of cell growth, were selected based on the DAPI signal. For each FOV, images were acquired with 0.5-micron z steps for twenty total slices. Integration of the automated fluidics system and imaging was controlled by a custom script written in uManager.

First, twelve hybridization rounds were imaged to capture all dynamic barcode states. The hybridization buffer for each round included two unique 15-nucleotide readout probes (See Supplemental Data of Chadly et al. incorporated by reference above) conjugated to either Alexa Fluor 647 (50 nM) or Alexa Fluor 546 (50 nM) in EC buffer (10% ethylene carbonate, 10% low molecular weight dextran sulfate, 4×SSC). Probes were allowed to hybridize for 15 minutes. Excess probes were washed away with 10% wash buffer (10% formamide, 0.1% Triton X-100 in 2×SSC) incubating for 1 minute. Nuclei were re-stained with DAPI solution (5 μg/mL DAPI in 4×SSC) incubating for 2 minutes. The sample was washed with 4×SSC and then imaged in anti-bleaching buffer (50 mM Tris-HCl pH 8.0, 300 mM NaCl, 2×SSC, 3 mM trolox, 0.8% D-glucose, 1000-fold diluted catalase, 0.5 mg/mL glucose oxidase). After imaging, readout probes were stripped off using 35% wash buffer (35% formamide, 0.1% Triton X-100 in 2×SSC). Although 55% formamide is typical for stripping readout probes, a lower concentration was used to avoid stripping primary probes and losing signal, as the primary probes (e.g., 20-nucleotides) were shorter than normal (e.g., 28-nucleotides) for dynamic barcode rounds. Images were collected after probe stripping to verify loss of signal. Due to occasional technical issues such as loss of focus during automated imaging, these twelve rounds were repeated a second time to collect backup images for each dynamic barcode round.

Static barcode sequences were captured by a similar scheme over twenty additional rounds of hybridization (See Supplemental Data of Chadly et al. incorporated by reference above for probe sequences), except using 55% formamide wash buffer to strip the readout probes. An additional six rounds of hybridization were carried out to capture the twelve gene markers described above. A final round of hybridization with wheat germ agglutinin (WGA) conjugated to Alexa Fluor 647 was carried out to stain cell membranes for downstream segmentation.

### Image Processing

Images were processed using custom Matlab scripts (See Supplemental Data of Chadly et al. incorporated by reference above). DAPI signal was measured in each round of imaging and used to register images across each hybridization round. After registration, z-stacks were projected by their maximum intensity to yield one image per channel per hybridization round for each colony.

Transcribed barcodes formed dots of variable intensity around the active site of T7 transcription. Dots were segmented using a combination of Laplacian of Gaussian filtering and watershed, requiring a maximum eccentricity of 0.8 to reduce noise. Loose parameters were chosen to detect all real dots at the expense of accepting some background noise. Binary images for each hybridization/channel were summed together to create a single mask, where pixel values represented the number of times that a pixel was identified across all imaging rounds. This single mask was termed “analog mask.”

Since each real dot should appear across all hybridization rounds in at least one channel, noise was further reduced by thresholding this image. A threshold was determined for each colony individually based on the elbow method. Segmentation errors were frequently observed, where the watershed algorithm was unable to separate adjacent dots from one another. These errors were manually corrected based on the analog mask, yielding a final binary segmentation mask of all detected barcode dots for each field of view.

DAPI segmentation masks were further generated using Ilastik to isolate individual cell nuclei. Masks were manually corrected using ImageJ to separate nuclei that were segmented together. Cells that intersected the border of the image were excluded. Any Zombie dots identified outside of cell nuclei were filtered. Dots may not be completely captured by the binary mask within any given round of imaging. A K-nearest neighbor classifier was used to partition all pixels belonging to each cell to the nearest dot in the segmentation mask so that intensity values could be extracted.

Raw images were background-subtracted to improve signal to noise. First, a tophat filter was used to globally reduce background. To further correct for variable intensity across images, local background correction was applied on a cell-by-cell basis by subtracting the median pixel intensity, which resulted in the excluded the dilated segmented Zombie dots.

Several features were extracted for each dot across all channels and hybridization rounds based on the background-subtracted raw images, taking the (log+1) of all intensity values. These features included total intensity, median intensity, 90^{th} percentile pixel intensity, pixel count, background median intensity and intensity variance.

A supervised machine learning approach was used to classify barcode states across each hybridization round. The barcode state was reflected in higher intensity fluorescence of probes that outcompeted other possible binders (FIG. 3A-FIG. 3F). Approximately 1000 randomly sampled barcode spots were manually classified for each image based on their pseudo-color intensities. Then, this sample was used to train a support vector machine (SVM) classifier in Matlab. Some spots were ambiguous, which were omitted in manual classification. 10-fold cross validation was carried out to evaluate model generalization and control for overfitting (FIG. 7A-FIG. 7E).

For dynamic barcode sites, the posterior probability was estimated for each spot belonging to each class under the SVM model. Many barcodes could be classified with high accuracy (e.g., >70% posterior probability), as shown in FIG. 3F and FIG. 7B. For static barcode sites, class assignments were compared to the whitelist of possible barcode sequences. Zombie spots were filtered out, if a character distance greater than 2 from an expected sequence and if the spots did not unambiguously correspond to a whitelisted static barcode. 79.3% of all detected spots were kept after filtering.

In many cases, duplicated barcodes were observed, where the same static barcode was identified multiple times in a single cell. These duplicates tended to be spatially localized and could be explained by either DNA replication or over-segmentation errors during analysis. For duplicated barcodes, classification probabilities were averaged at dynamic barcode sites and the most confident state was used for downstream lineage reconstruction.

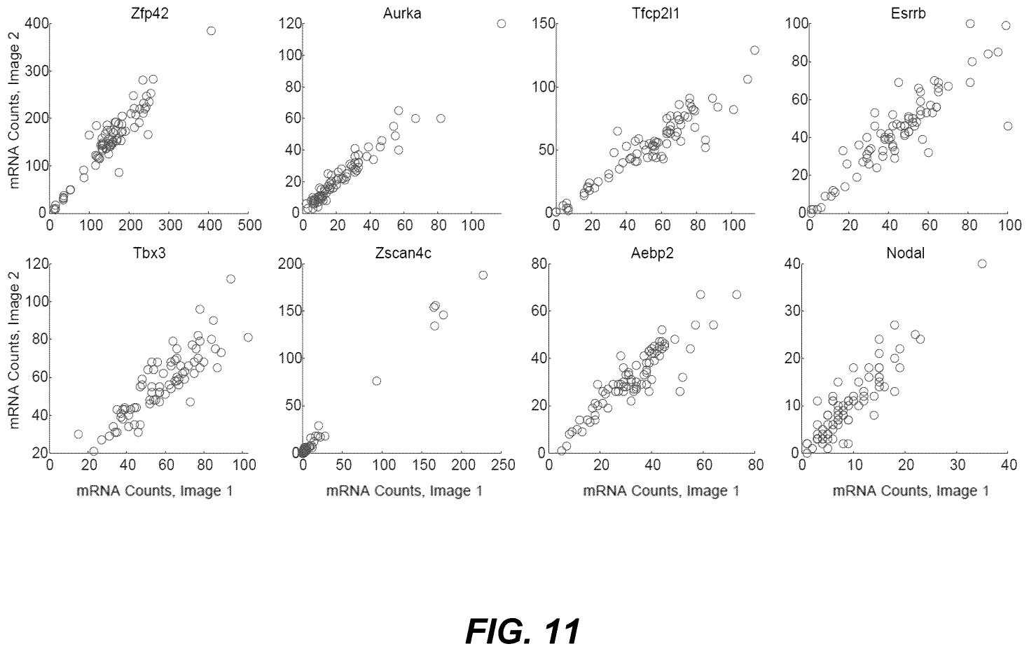

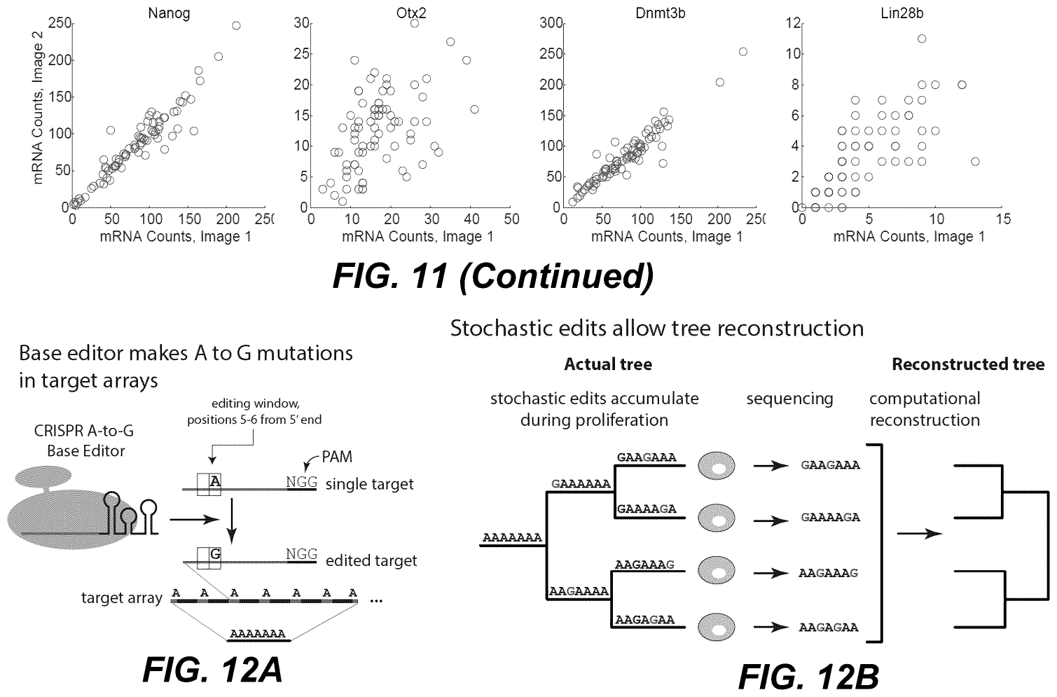

Membrane masks were manually generated based on WGA staining images. Then, gene markers were identified using the bigFISH package dot detection method. Thresholds for dot detection were manually determined for each gene. Segmented spots, corresponding to mRNA molecules, were tallied within each cell as defined by the membrane segmentation mask. To validate the consistency of this method, the detection frequency was plotted for each gene across all cells that were measured in multiple images (FIG. 11). The measures were highly correlated.

### Cell Type Analysis

Cell types were determined by using k-means clustering on log transformed mRNA counts with 5 centers to group the most distinct sets of cells in the dataset. Dimensionality reduction using the tSNE method visualized three well-separated groups and three of the identified cell states (e.g., Naive/2C-like, Naive and Naive/Formative) as potentially a continuous distribution, although dimensionality reduction techniques could obscure the true distances between cells and clusters in the higher dimensional transcriptome space. Most importantly, unique allowed and forbidden transitions between each purported cell state were identified through subsequent lineage analysis that was agnostic to the underlying transcriptional information, bolstering the claim that these five clusters of cells should be treated as distinct populations.

### Lineage Reconstruction and Bayesian Modeling

A Bayesian model was used under the BEAST2 v2.7 framework that took barcode information, end point cell labels and cell centroid positions as input to jointly estimate lineage relationships, cell state transition dynamics and cell motility.

Barcode information for each dinucleotide was extracted using Matlab and R scripts (See Supplemental Data of Chadly et al. incorporated by reference above). Each of the four dinucleotide states (AA, AG, GA and GG) was encoded in a single character (A, T, C or G). Characters that were not recovered during imaging were marked as missing data by the “?” character. Cell division for each tree was modeled as a pure birth process (the Yule model) with birth rate estimated.

Barcode character mutation in the herein disclosed system was irreversible. With few exceptions, existing BEAST2 packages only model reversible character transitions because these packages made computing tree likelihoods more computationally efficient. A new irreversible character substitution model was developed to better capture the evolutionary process that generated the data disclosed herein, which is available as the ‘irreversible’ package for BEAST2, with source code available from github.com/rbouckaert/irreversible. Under this model, each possible transition (AA to AG, GA or GG) can take a unique rate value, which was constant along the tree. Stationary frequencies, which were used at the root of the tree to calculate the tree likelihood, were set at 1 for the AA state, and 0 for the others, reflecting that every state was AA at the root of the tree. Since targets can be edited at different rates and into different outcomes, the rate was allow to vary across sites through the gamma site heterogeneity model, partitioning the allowable rates into 4 categories. This model was shared across all trees. Furthermore, a strict molecular clock was used since significant rate variation per branch was not expected.

Cell state transitions were modeled as a continuous time Markov chain with symmetrical transition rates possible between each state. These rates were assumed to be constant across time with an associated strict clock model. Transition rates were shared between all trees. Thus, a single unified cell state transition model was estimated across all colonies. Symmetric transition rates were assumed based on previous work, which identified most transitions as roughly symmetric in this system. In principle, the assumption can be relaxed, although it greatly increased the number of parameters in the model, thus increasing susceptibility to overfitting.

Cell motility was modeled as single parameter 2D diffusion along the surface of a sphere. Spherical diffusion was a good approximation of diffusion in a 2D plane for small patches of the surface and its implementation was efficient. Accordingly, cell positions were mapped to geographical coordinates falling within 2 latitude and longitude degrees. The diffusion parameter describing motility was allowed to take unique values along each branch of the tree under a relaxed clock model.

Notably, all 7 colonies in the dataset disclosed herein were analyzed simultaneously under a single model. This method allowed inference of barcode character substitution and cell state transition models that were shared across all the data, reflecting that all colonies were representative of the same underlying barcode mutation and cell state transition processes. This is reasonable given that all colonies were generated from a monoclonal culture grown in identical culture conditions over the same time period.

Priors were chosen to be uninformative with the exception of the root height, since prior information strongly suggested that experiments lasted 3 days. An uninformative but improper uniform distribution across all possible rates was chosen for barcode mutation rate, although this was not expected to affect the resulting analysis or MCMC mixing. Detailed prior information was recorded as shown in FIG. 5A-FIG. 5H specifying all modeling choices.

### Movies

Supplementary movies were generated by creating inferred still images of the maximum a posteriori histories of cells over time, incorporating inferred ancestral cell states, positions, and cell division timings (Supplemental Data). These still images were compiled into movies using the open-source video editor Shotcut (Meltytech).

### Lineage Motif Analysis

The posterior baseMEMOIR trees were analyzed using Lineage Motif Analysis (LMA), using the resample_trees_doublets, resample_trees_triplets and resample_trees_quartets functions with 1000 resamples. These functions were available in the publicly available “linmo” package for Python (github.com/labowitz/linmo). To generate a z-score and adjusted p-value for all cell fate patterns across the entire posterior distribution of each tree dataset, 1000 synthetic datasets were generated by randomly drawing one tree from the posterior distribution of each tree dataset. Each synthetic dataset therefore contained 7 total trees. LMA was then performed on each synthetic dataset. The distribution of z-scores and adjusted p-values were plotted for each cell fate pattern.

### Example 2

### Regenerative Editing for Molecular Recording

### Background

Reconstructing lineage relationships among related cells is a fundamental challenge in biology. Multicellular organisms arise from a single cell, which divides and differentiates to produce a variety of cell types that implement each of the specific functions necessary for life. Foundational studies in C. elegans determined, through comprehensive microscopy, that each cell in the organism originated through a deterministic lineage that was identical across individuals. In most other organisms, however, the situation is more complex. Lineage relationships are not typically deterministic and vary substantially among individuals. Except in rare cases, the set of paths a single cell can take to produce an organism, either in healthy individuals or under the stresses of disease, remains incompletely understood. Lineage determination is also more challenging in many relevant model organisms, which are typically larger and non-transparent, preventing analysis by direct microscopic observation. Organoids provide another set of biomedically important systems in which understanding lineage relationships could be valuable. New tools are needed to understand how largely stereotypic organisms can result from non-stereotypic lineage relationships at the single cell level.

To address these challenges, several synthetic systems have been developed, which dynamically edited (mutate) specific genomic regions as cells proliferated, and subsequently read out those edits by sequencing or imaging to reconstruct lineage from endpoint measurements. For example, in MEMOIR systems, designed DNA sequences, termed “scratchpads,” were integrated into the genome and then stochastically and irreversibly edited by CRISPR/Cas9 or an integrase over multiple cell cycles. The edits were read out by imaging, allowing in situ recovery of edit patterns from individual cells, and reconstruction of lineage trees. Systems such as GESTALT, CARLIN, LINNAEUS, SMALT, and the homing CRISPR barcoded mouse, also used CRISPR/Cas9 to edit designed target sequences, relying on next generation sequencing to read out edited barcodes. Alternative systems, including CAMERA, leveraged CRISPR base editors to generate more specific types of barcode diversity. Regardless of the type of edits or the readout method, lineage relationships between individual cells can be reconstructed from each cell's unique pattern of target site edits in a manner analogous to sequence-based phylogenetic reconstruction. Most recently, barcode labeling by incorporating temporally ordered sequences using Prime Editing technology had been introduced as a new paradigm for phylogenetic recording.

To date, most lineage tracing systems have focused on the recovery of coarse grained lineage relationships over long timescales, with notable exceptions of the MEMOIR and SMALT systems, which have attempted to recover lineages with high temporal resolution. Several Features limit our ability to reconstruct deep lineage trees at single cell cycle resolution in developing tissues and organisms: one major limitation in current phylogenetic systems is their memory capacity. Further, reconstruction accuracy in most phylogenetic systems is limited by non-linear use of memory over time. For example, at a constant edit rate, an exponential loss of target sites is expected as time progresses, leading to a disproportionate number of marks being made in the genome at early time points, or effectively “front-loading” the edits to the earliest cell cycles and depleting the store of remaining unedited sites that can be subsequently edited.

In addition to the limitations described above, there are technical limitations in the ability to engineer phylogenetic recording systems into cells. Most systems require extensive engineering and potentially clonal selection steps, which are possible only in limited cases with cell lines that are amenable to in vitro culture. Many cell types, especially primary cells, can only be cultured out of their host organism for a short time, limiting the ability to engineer in synthetic systems. Thus, there is an unmet need to generate a phylogenetic recording system that can record lineage deeply and be introduced to cells quickly and easily.

### Summary

A new system, termed the “hypercascade,” is disclosed herein. The hypercascade densely packs editable target sites together in a four-level cascade, with editing of a given site at one level dependent on editing of two specific sites at the previous level. This design achieves extended durations of editing with near-linear barcode loss over time and allowing convenient single cell readout. Further, because it is compact, this system can be introduced to human induced pluripotent stem cells in a single step via piggyBac transposition. Simulations revealed relative insensitivity of the system to edit rate and target array copy number, which, in turn, allowed deep lineage reconstruction even in polyclonal populations of cells. Based on these results, the hypercascade recorder enables detailed lineage reconstruction in otherwise inaccessible model systems, and allows reconstruction of additional signals.

### Description

### The Hypercascade Allows Sustained Editing Through Sequential Generation of Target Sites

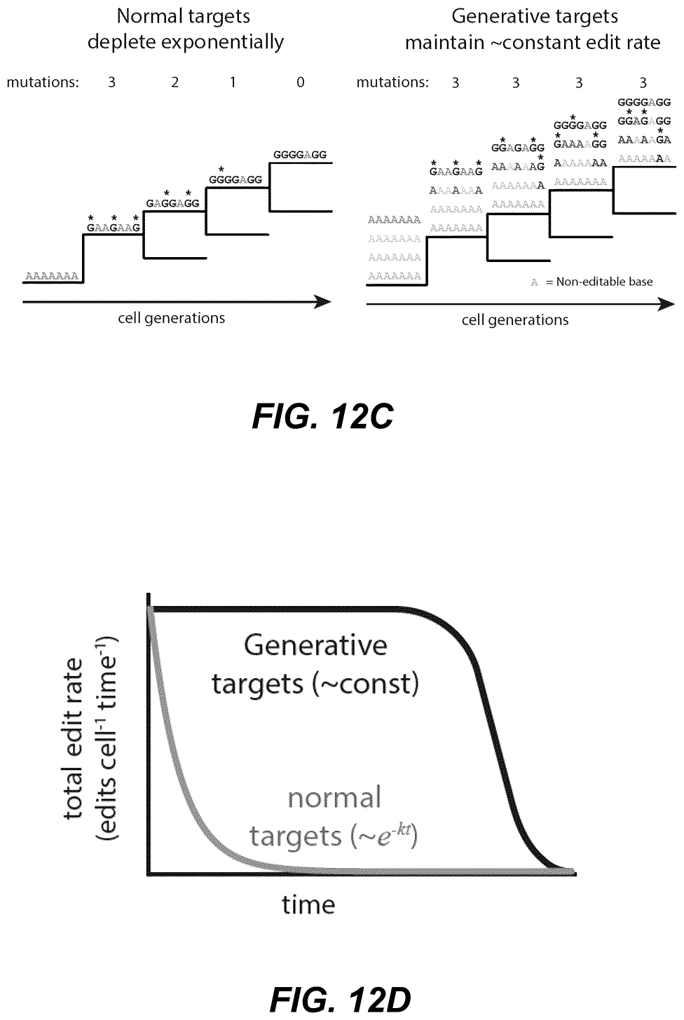

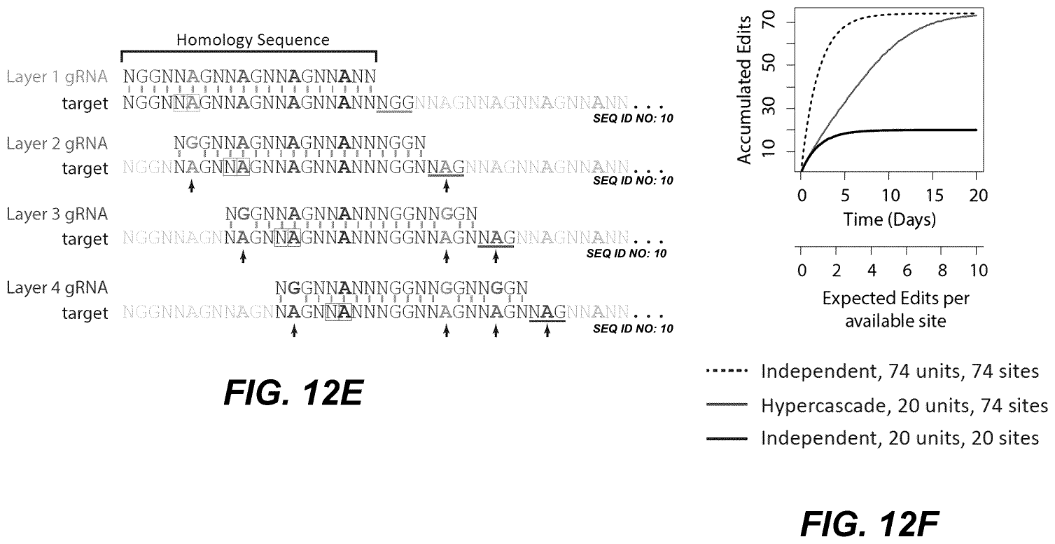

Reconstruction of deep lineages requires a set of editable sequence barcodes (e.g., memory elements) that can be read out in individual cells. Emerging technologies, such as the CRISPR A-to-G base editor (ABE), enable specific mutation of arrayed target sites, encoding information into the cellular genome (FIG. 12A). This information can be used to recover lineage relationships between dividing cells, taking advantage of the heritability of mutations (FIG. 12B). An ideal target array would have three key features: first, in order to maximize the amount of memory it could encode, and thereby make reconstruction as accurate as possible, it should exhibit a high density of editable target sites; second, to facilitate ease of use, integration into genomic targets, and portability to a wide range of cell systems, it should be as compact in total length as possible and easily integrated into the genome; and third, and most uniquely, in order to accumulate informative edits uniformly over time and avoid exponential memory loss, new target sites would be generated as they are consumed (FIG. 12C). A system that can meet these goals was developed, taking advantage of unique properties of the ABE.

The ABE has two key structures for efficient function. One is a 20-base pair homology sequence encoded by the gRNA, with which it complexes. The other is a 3-base pair protospacer adjacent motif (PAM), which is NGG for the most commonly used Cas9 homolog derived from S. pyogenes. When these two requirements are met, an A in the fifth or sixth position of the target sequence is mutated to a G (FIG. 1A). Since base editing generates a predictable G on the target strand, it is possible to create new targets as edits accumulate by repairing mismatches that inhibit editing mediated through alternative gRNAs (FIG. 12D). Taking advantage of this feature, a cascading system was designed, which packs four editable target sites within one repeating 20 bp sequence that can be operated on using four unique gRNAs (FIG. 12E). There were 11 degrees of freedom in the design of the repeating element, yielding a total of around 4 million possible sequences that can operate in this manner.

In the top layer of the proposed system, PAM sites already existed for a first gRNA arrayed every 20 base pairs. These targets were always available for editing (FIG. 12D). Three further layers of gRNAs were nested within this sequence that were activated by edits occurring on upper layers. For a deeper layered gRNA to be activated, edits must have already been made in both adjacent target sites at the upper level (FIG. 12D and FIG. 12E). This system, which resembles a 2-dimensional array of linked cascades, is termed a “hypercascade” (FIG. 12E). This arrangement allows dense packing of editable bases while requiring only a small number of gRNAs for operation.

In addition to densely packing many targets into a small length of sequence, the cascading feature has the benefit that, on average, barcode use over time is nearly constant. With fully independent barcodes, the number of subunits available for editing decreases exponentially over time, leading to fewer edited sites in later generations and a decreased ability to assign lineage or record signals (FIG. 12C). Editing in the hypercascade, on the other hand, generates new targets as editing progresses, leading to a linearization of edit rate over the entire array across time.

Stochastic simulations were used to compare editing in independent arrays with hypercascading arrays of comparable edit rate per accessible target site (FIG. 12F). Hypercascade editing with 20 repeating units initially resembled a 20-unit independent array, but then accumulated edits roughly linearly until it began to saturate when all 4 layers were exhausted. For a given edit rate, this extended the duration of recording compared to independent recorders, which saturated earlier regardless of array length. Importantly, the 20-unit hypercascade had the same array length in DNA base pairs as a 20-unit independent array, a key engineering feature.

### Simulations Show that the Hypercascade Enables More Accurate Reconstruction Compared to a Simple Array

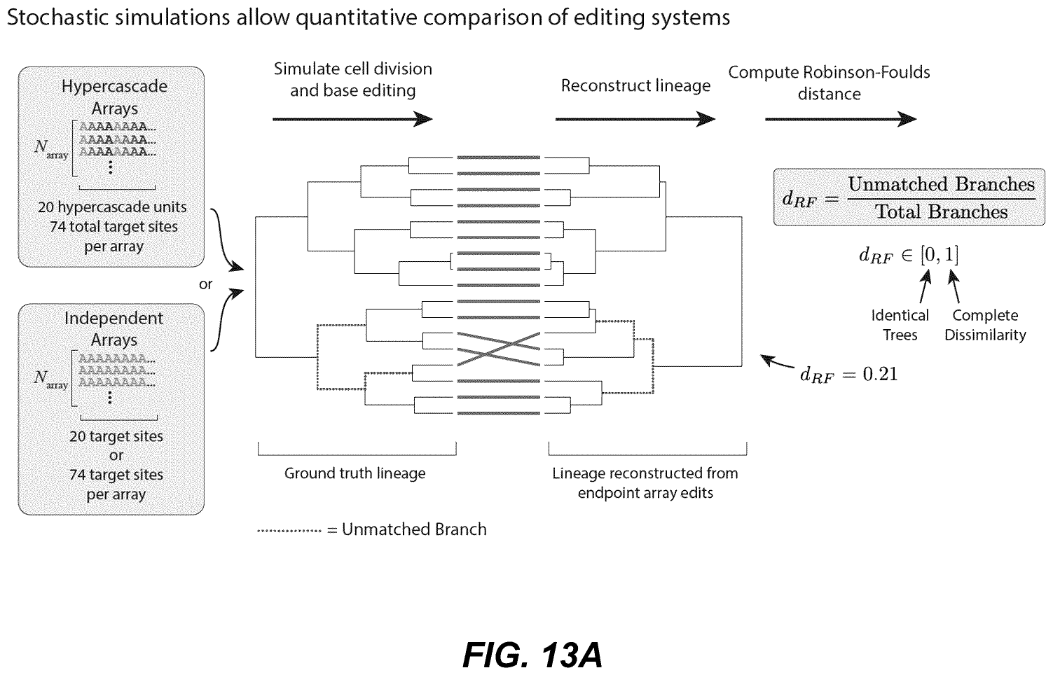

To better understand the impact of hypercascade editing on lineage reconstruction, both cell division and barcode editing were simulated with either hypercascading or independent target arrays (FIG. 13A). The hypercascade length was fixed to 20 repeating units, which included 74 total targets across the four layers, representing a 400 bp insert. Then, the hypercascade was compared to independent arrays under two limiting scenarios: 1) an independent array of comparable sequence length (e.g., 400 bp), which would contain 20 total target sites and 2) an independent array with an equivalent number of total targets (e.g., 74 targets), which would necessarily have a much longer sequence length (e.g., 74 repeating units, 1.48 kb). In the first case, the combined benefits of increased target density and edit rate linearization in hypercascade editing were observed. In the second, the increased target density of the hypercascade was controlled and the benefits of edit rate linearization alone was investigated.

Stochastic editing was simulated in both systems across a range of edit rates, array copy numbers, and tree depths. After simulating trees in the forward direction, barcode states were used to reconstruct lineage relationships between the cells retroactively. Reconstructed trees were compared to ground truth trees using the normalized Robinson-Foulds distance, in which 0 represented a perfect match between the trees and 1 represented the maximum possible number of differences (FIG. 13A).

To comparing the hypercascade to an independent array of equal length, the mean tree depth was fixed at 10 cell divisions, while both edit rate and array copy number were varied (FIG. 13B). At low edit rates, the hypercascade performed comparably to a 20-unit array. This was as expected, because the first layer of the hypercascade was essentially an equivalent to 20 independent units. At higher edit rates, two main benefits of hypercascade became clear. First, high reconstruction accuracy was attainable for relatively few integrations of the hypercascade, even with the challenging task of reconstructing a 10-cell division tree. Second, the hypercascade was relatively insensitive to edit rate. This feature was enabled by the new target sites generated as the old sites depleted, linearizing the loss of targets over time and preventing the exponential loss of memory characteristic of independent units (FIG. 12D).

The impact of time linearization on editing outcomes was also determined, disregarding target density. 74-target hypercascade arrays were compared with independent arrays with the same total number of targets (FIG. 13C). In this case, the target copy number was fixed to 3 in both cases, while both edit rate and mean tree depth were varied. Because fully independent targets were uncorrelated, they had the potential to contain more information than hypercascade targets, in which some barcode states were never realized due to the ordered nature of editing. This was reflected in the relative performance of the two systems at low edit rates, especially with increasing tree depth as reconstruction became more challenging. At low edit rates, the independent array typically reconstructed lineage more accurately than the hypercascade in this case. Interestingly, however, the hypercascade overcame this disadvantage at higher edit rates and deeper trees over a wide range of parameters, in which exponential saturation of the linear arrays had greater impact on the outcome than the benefits of uncorrelated editing.

### Different Hypercascade Sequences Operate Orthogonally with Distinct Kinetics

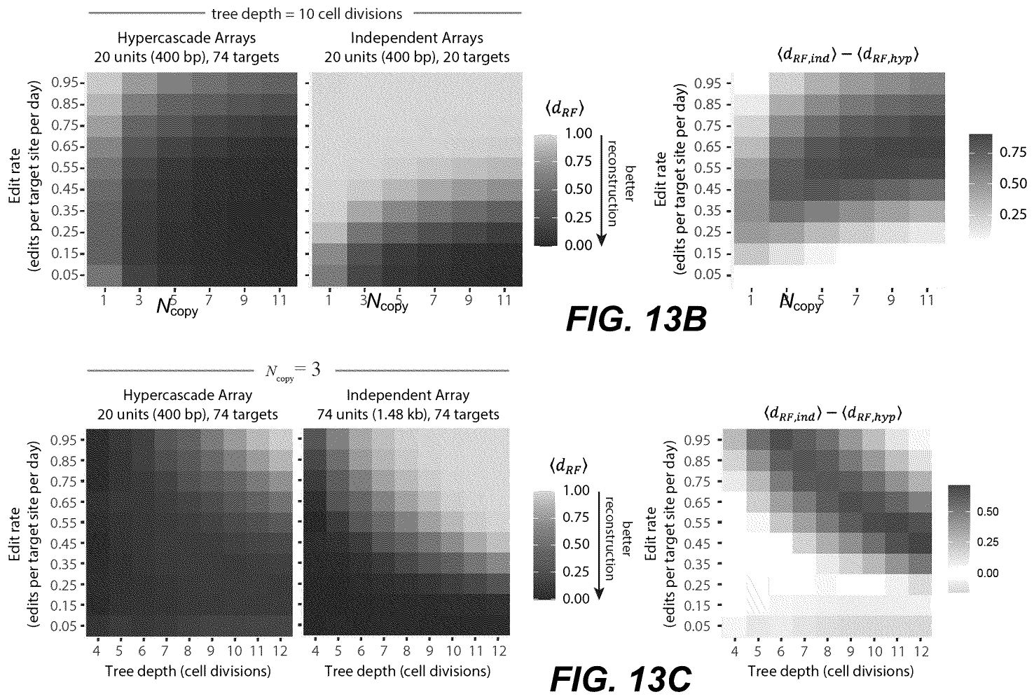

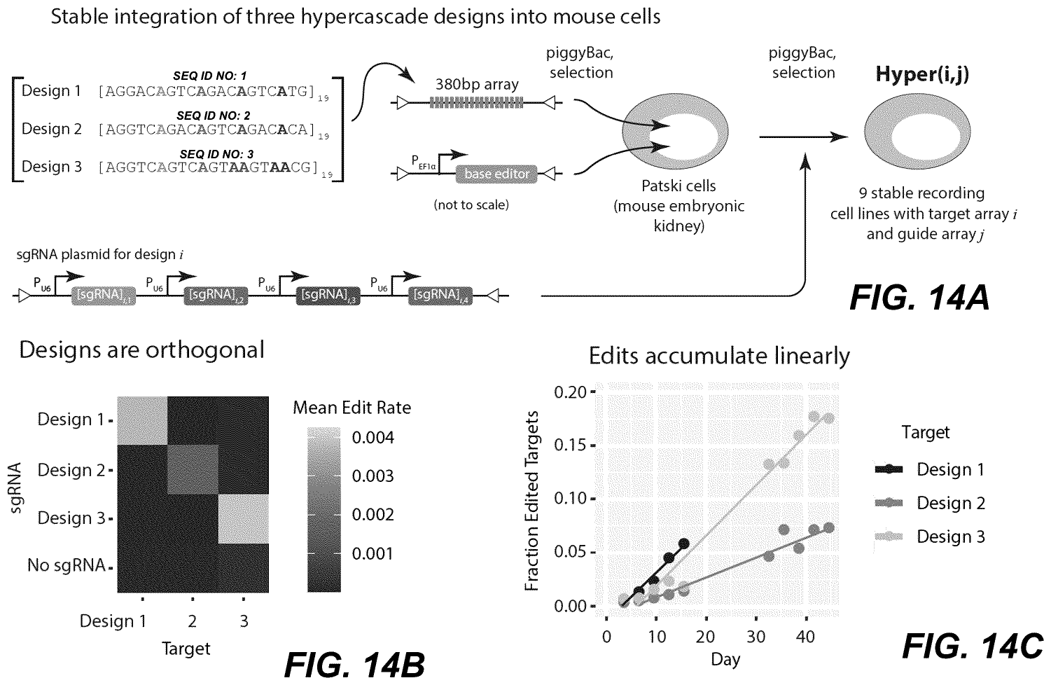

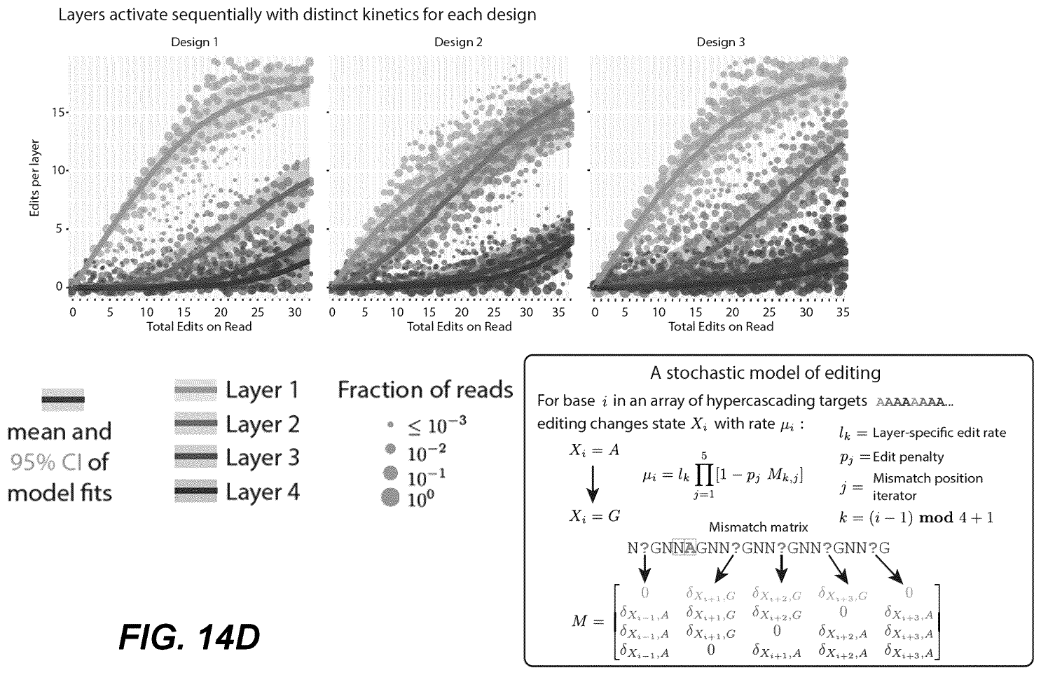

To assess the functionality of the barcode design, three different sequences were integrated with the hypercascading property and base editing was induced (FIG. 14A). Each sequence contained 19 tandem repeats of the described 20-mer, choosing different bases at the free locations for each sequence based on predicted editing efficiencies of each layer based on an existing machine learning model (FIG. 14A). The barcode arrays were integrated alongside the ABE into a mouse embryonic kidney fibroblast cell line via piggyBac transposase. Then, the engineered cells were selected with antibiotic to generate polyclonal lines, each containing one specific target sequence. Editing was initiated by integrating gRNA expression constructs on day 0, allowing editing to progress during a 44-day period. To investigate the orthogonality of the system, gRNAs and targets were tested together in all combinations.

The fraction of edited sites increased over time for all three target sequences, with no editing observed in the mismatched gRNA-target combinations (FIG. 14B and FIG. 14C). To investigate the order of edit accumulation on each read, reads from all time points were compiled and sorted by total edits on the read. The distribution of edits per layer for reads with different numbers of total edits demonstrated that editing in different layers occurred generally in sequential fashion (FIG. 14D).

### The Entire System can be Delivered Via One Step Transfection

The hypercascade enables a new paradigm for deep lineage recording. Rather than laboriously engineering cells to express multiple components, screening monoclones, and potentially repeating until cell lines with the desired characteristics can be generated, the present system enables simultaneously integrating all components via piggyBac transposition, performing a single antibiotic or fluorescence-based selection step, then moving forward immediately with downstream experiments using the resulting polyclonal cell line. Although different clones can edit at different rates or have different target copy numbers, the hypercascade system offered insensitivity to both parameters (e.g., different edit rates and target copy numbers) (FIG. 13A-FIG. 13C). This approach enables recording in systems that are traditionally difficult to engineer or prone to silencing, including primary cells and human induced pluripotent stem cells (hPSCs).

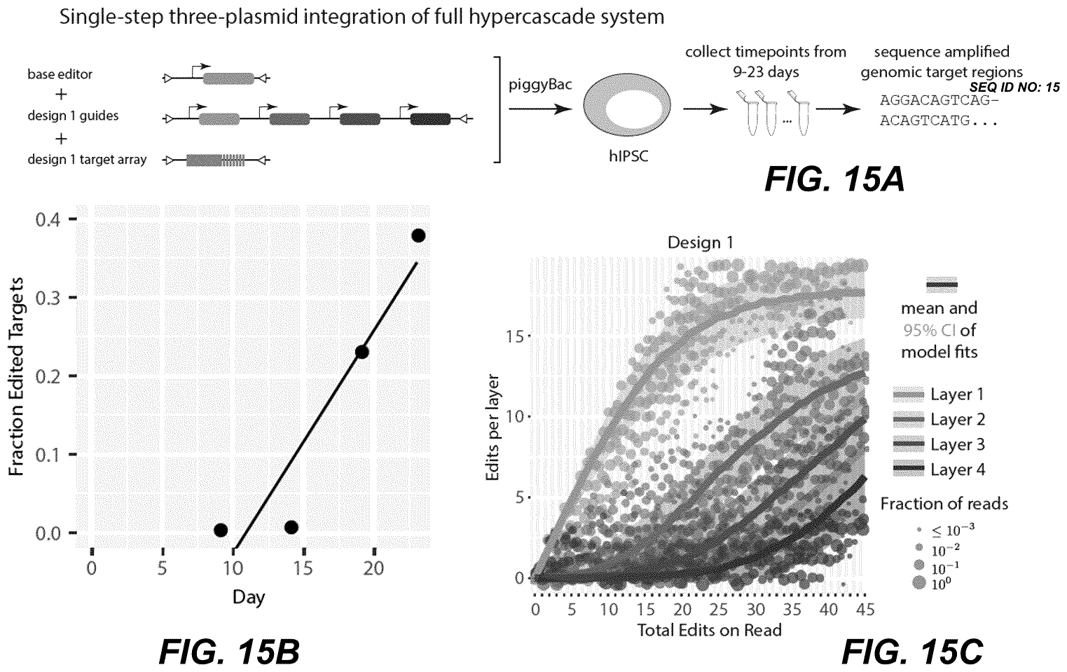

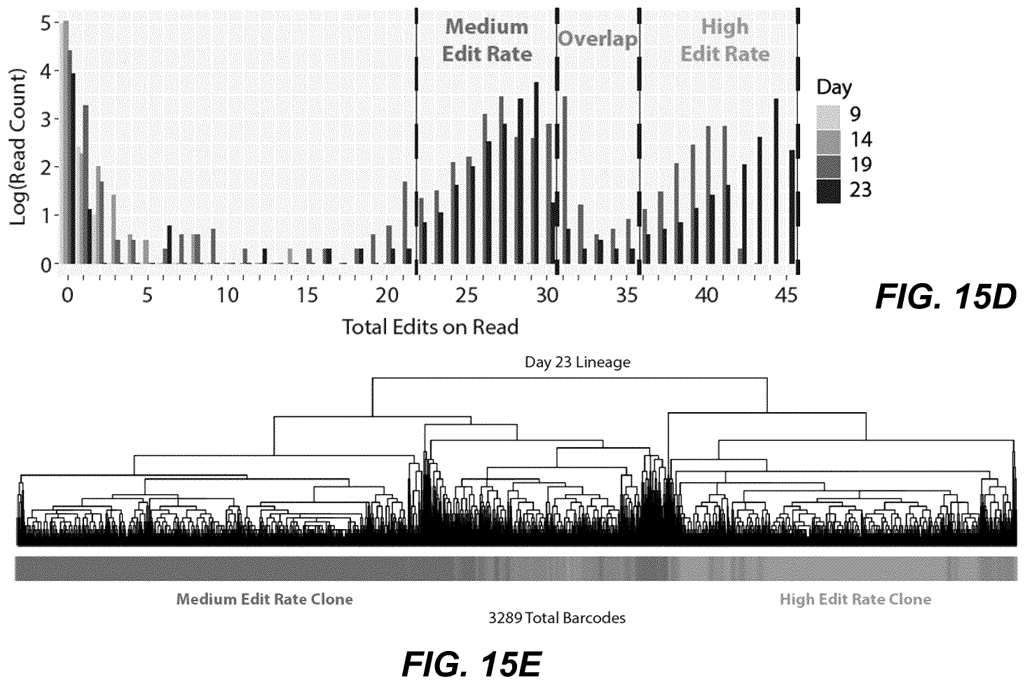

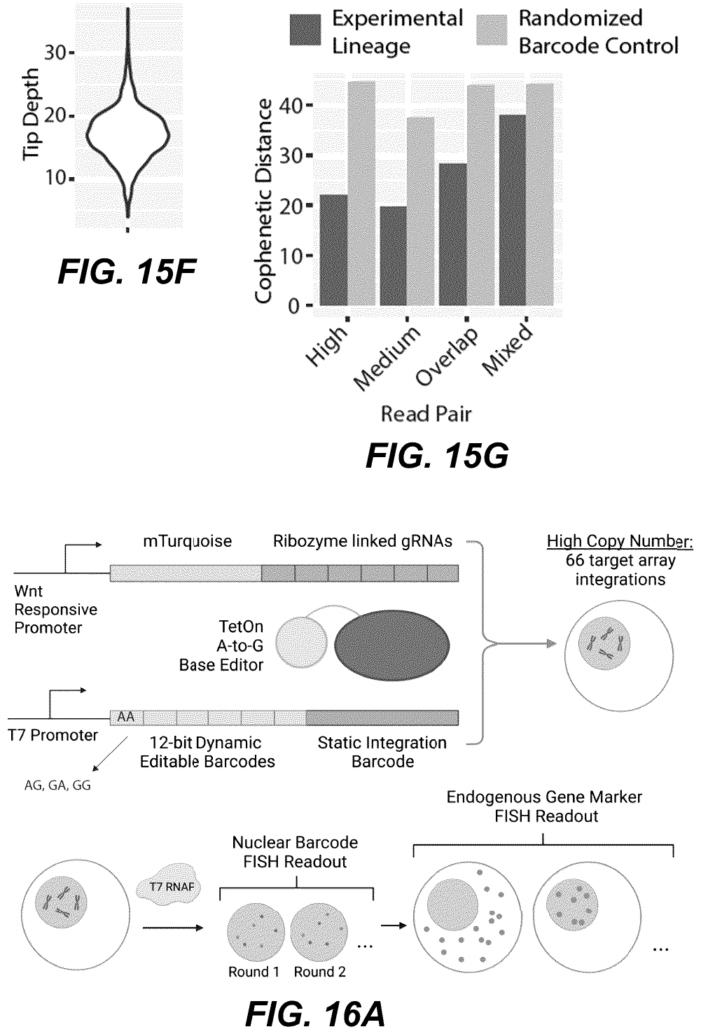

This approach was investigated using hypercascade target sequence 1 by simultaneously transfecting all components into hiPSCs. The engineered hiPSCs were selected with antibiotic for 1 day, and then continuing cultured. Samples were collected every passage for genomic DNA extraction once they recovered (FIG. 15A). With this approach, edits accumulate over time was observed after an initial delay period (FIG. 15B). This delay was likely the result of lingering plasmid from the transfection that was slowly diluted out over time. After the initial delay, edits accumulate through a 23-day period post transfection was observed (FIG. 15B). The distribution of edits into different layers indicated the sequential activation of layers in the system, similar to that observed in the mouse cells engineered over several steps (FIG. 15C).

Interestingly, the frequency of reads with different total numbers of edits naturally grouped into several distinct distributions (FIG. 15D). These could represent individual clones or groups of clones that received specific copy numbers of the editing component plasmids, leading to different edit rates. Distributions shifted to the right over time, which was as expected in the context of genomic edit accumulation. Unique edit patterns were identified within the data and reconstructed lineage relationships, leaving out targets with few edits that contain relatively little recorded information (FIG. 15E). Medium and high edit rate cells clustered together into clades, a feature that was not retained in a scrambled barcode control (FIG. 15G). The depth of the reconstructed tree was similar with regard to reads generated from both medium and high edit rates, and was only slightly smaller than the number of generations expected for hiPSCs during the 23 days of recording, while roughly 23 cell divisions was anticipated (FIG. 15F).

### Example 3

### Molecular Recording Systems Capturing Lineage Relationships in Dividing Stem Cells

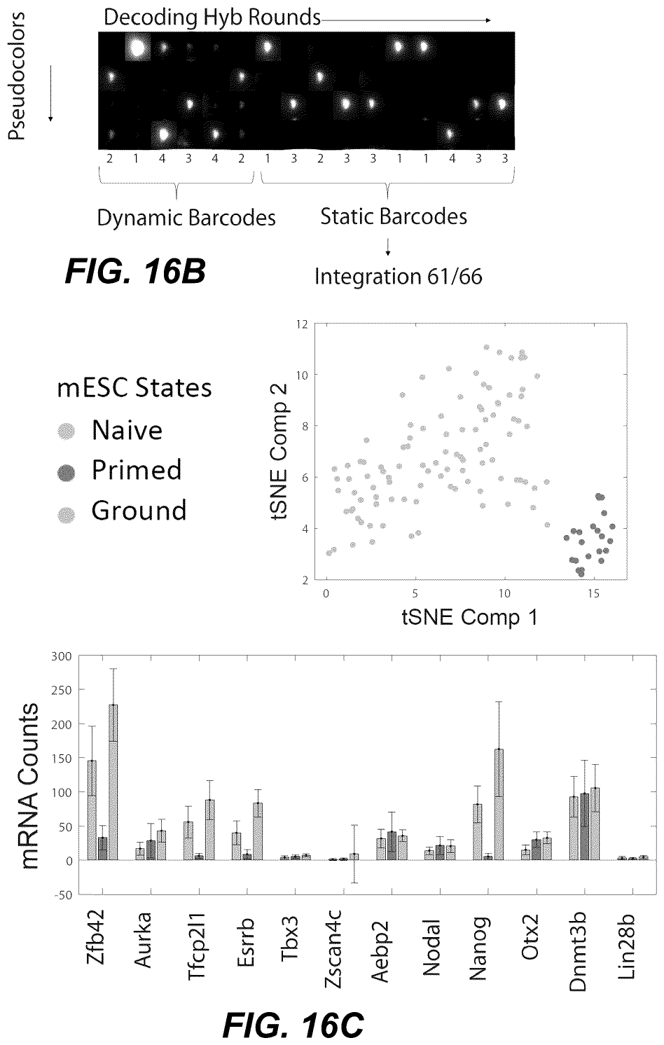

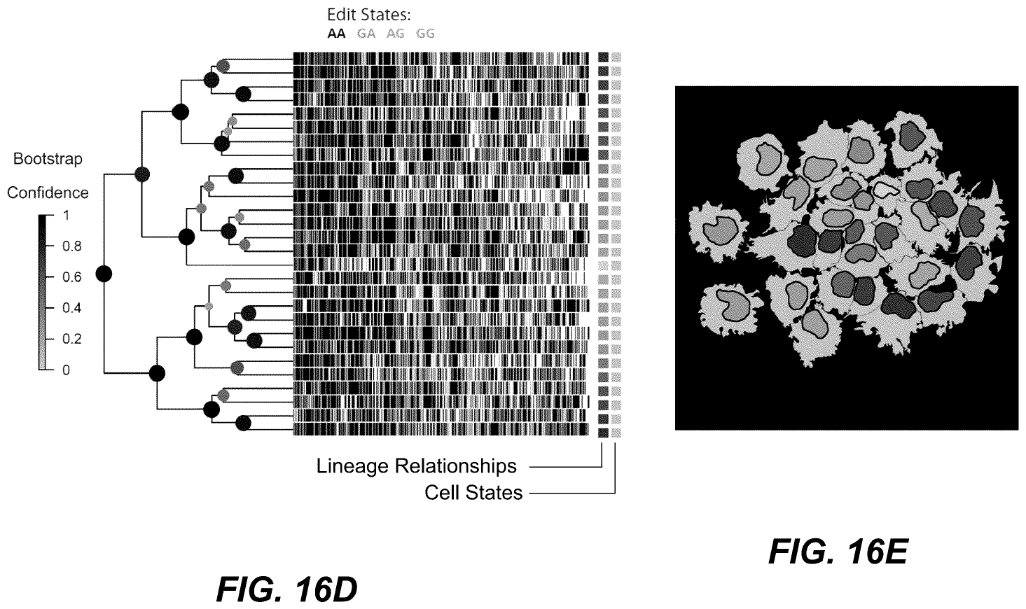

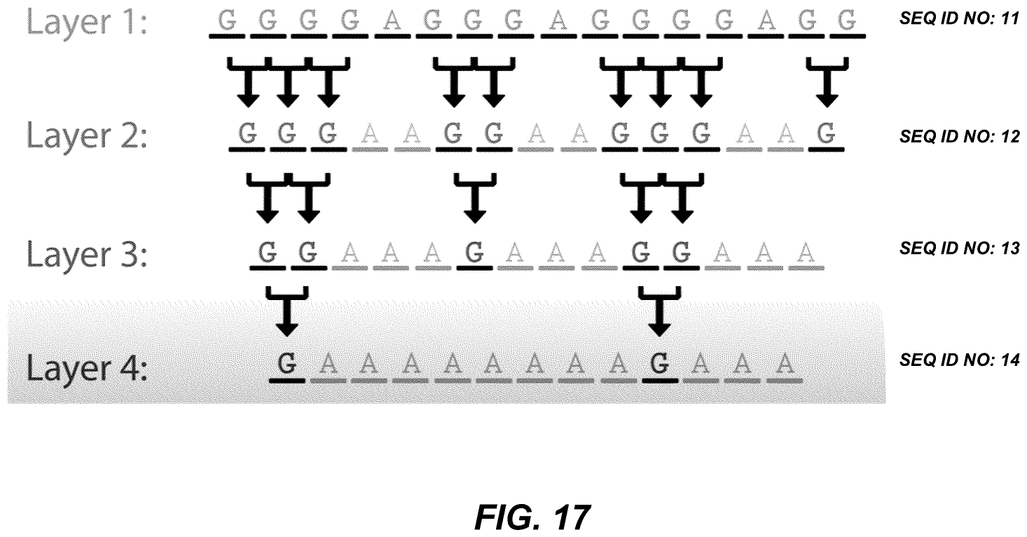

The BigMEMOIR (e.g., baseMEMOIR) mESC line was generated which contains integrated synthetic DNA sequences which have dynamically editable barcodes as well as identifying static barcodes (FIG. 2A). Dynamic barcodes are edited by the CRISPR A-to-G base editor complexing single guide RNAs (sgRNAs). Base editor and sgRNAs were introduced on separate constructs and are expressed by inducible promoters, allowing controllable activation of the system. All constructs were genomically integrated via piggyBac transposase preceding monoclonal selection. Barcode states and endogenous mRNAs are detected using in situ transcription and fluorescence in situ hybridization. FIG. 16A-FIG. 16E depict non-limiting exemplary embodiments and data related to the BigMEMOIR (e.g., baseMEMOIR) line containing editable barcodes. FIG. 16A depicts that the editable barcodes allowed for sequential FISH readout analysis. FIG. 16B depicts that fluorescent images of barcodes collected across multiple rounds of hybridization obtained using FISH techniques were categorized by machine learning to identify the brightest fluorescence against four potential pseudo-colors, decoding dynamic and static barcode sequences. FIG. 16C depicts that cell states were clustered by expression of pluripotent stem cell state markers. Cell state data was paired with lineage data to correlate cell state relationships with lineage and physical location. FIG. 16D depicts that long barcodes with 396 characters were determined for each cell, combining information from all target array copies. Dynamic barcodes allowed for reconstruction of phylogenetic lineage relationships. In FIG. 16E, the colony is colored based on cell state (pink and orange) as well as relative placement on the phylogenetic tree (red to blue), with warm colors and cool colors representing earlier splits into clades. More subtle shades within a hue represent more closely related cells.

### Conclusions

Provided herein are two systems for lineage reconstruction using hiPSCs and mESCs. The BigMEMOIR (e.g., baseMEMOIR) cell line allows for the retention of spatial data through the use of fluorescent in situ hybridization imaging. Recording with the hypercascade design (See Example 2) enables phylogenetic reconstruction with long term data collection facilitated by the layered editing approach. While both systems record lineage information, each is specialized in what kind of data it can obtain. The BigMEMOIR (e.g., baseMEMOIR) system can be used in the future to investigate lineage correlations in an in vitro model of mouse early development. Hypercascade uses can include observing lineage trends in differentiating cells such as cardiomyocytes.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A system, comprising:
(i) one or more hypercascade array(s) each comprising p hypercascade units, wherein p is an integer greater than 1,
(ii) n layer guide RNAs (gRNAs), wherein n is an integer greater than 1; and
(iii) an editor,
wherein each hypercascade unit comprises m target sites, wherein m is an integer greater than 1, wherein the target sites comprises one or more primary target site(s) and one or more conditional target site(s),
wherein target sites each comprise an editable base, wherein the editor associated with a layer gRNA is capable of editing said editable base of a target site comprising a complementary protospacer and a protospacer adjacent motif (PAM),
wherein the primary target sites are capable of being edited by a first layer gRNA and the editor,
wherein each of the one or more conditional target sites comprise two or more mismatches in the protospacer and/or the PAM, and
wherein the editing of adjacent editable bases by a previous layer gRNA are capable of repairing said mismatches, thereby enabling the editing of a conditional target site by a layer gRNA and the editor.

2. (canceled)

3. The system of claim 1, wherein each hypercascade unit comprises the same length and/or sequence.

4. The system of claim 1, wherein each hypercascade unit comprises an identical N-mer, wherein the N-mer is 15, 16, 17, 18, 19, 20, 21, 22, 23, 24, 25, 26, 27, 28, 29, 30, 31, 32, 33, 34, 35, 36, 37, 38, 39, or 40, nucleotides (nt) in length.

5. The system of claim 1, wherein:
the two or more of the p hypercascade units are in tandem; and/or
the hypercascade array comprises a tandem repeating 20-mer.

6. (canceled)

7. The system of claim 1, wherein:
the hypercascade unit comprises a nucleotide sequence that is at least 80%, 85%, 90%, 95%, 98%, 99%, or 100% identical to (NGGNNAGNNAGNNAGNNANN);
the hypercascade unit comprises a nucleotide sequence that is at least 80%, 85%, 90%, 95%, 98%, 99%, or 100% identical to SEQ ID NO: 1 (AGGACAGTCAGACAGTCATG);
the hypercascade unit comprises a nucleotide sequence that is at least 80%, 85%, 90%, 95%, 98%, 99%, or 100% identical to SEQ ID NO: 2 (AGGTCAGACAGTCAGACACA); and/or the hypercascade unit comprises a nucleotide sequence that is at least 80%, 85%, 90%, 95%, 98%, 99%, or 100% identical to SEQ ID NO: 3 (AGGTCAGTCAGTAAGTAACG).

8. (canceled)

9. (canceled)

10. (canceled)

11. (canceled)

12. The system of claim 1, wherein:
each hypercascade unit comprises m−1 conditional target sites capable of being activated by adjacent edits mediated by an upper layer gRNA;
the editable base is situated at the fifth or sixth position of a target site; and/or
the editable base comprises adenine.

13. (canceled)

14. (canceled)

15. The system of claim 1, wherein the repair of repairing protospacer and PAM mismatches through A-to-G edits of a previous layer gRNA enable the editing of conditional target sites.

16. The system of claim 1, wherein the n layer gRNAs comprise:
a first layer gRNA;
a second layer gRNA;
a third layer gRNA; and/or
a fourth layer gRNA.

17. The system of claim 16, wherein:
the first layer gRNA associated with the editor is capable of editing the primary target sites;
the second layer gRNA associated with the editor is capable of editing first conditional target sites upon repair of adjacent mismatches by the first layer gRNA associated with the editor;
the third layer gRNA associated with the editor is capable of editing second conditional target sites upon repair of adjacent mismatches by the second layer gRNA associated with the editor; and/or
the fourth layer gRNA associated with the editor is capable of editing third conditional target sites upon repair of adjacent mismatches by the third layer gRNA associated with the editor.

18. (canceled)

19. (canceled)

20. (canceled)

21. The system of claim 16, wherein:
the first layer gRNA comprises a nucleotide sequence that is at least 80%, 85%, 90%, 95%, 98%, 99%, or 100% identical to (NGGNNAGNNAGNNAGNNANN);
the second layer gRNA comprises a nucleotide sequence that is at least 80%, 85%, 90%, 95%, 98%, 99%, or 100% identical to (NGGNNAGNNAGNNANNNGGN);
the third layer gRNA comprises a nucleotide sequence that is at least 80%, 85%, 90%, 95%, 98%, 99%, or 100% identical to (NGGNNAGNNANNNGGNNGGN); and/or
the fourth layer gRNA comprises a nucleotide sequence that is at least 80%, 85%, 90%, 95%, 98%, 99%, or 100% identical to (NGGNNANNNGGNNGGNNGGN).

22. (canceled)

23. (canceled)

24. (canceled)

25. The system of claim 1, wherein the layer gRNA is a single guide RNA (sgRNA).

26. The system of claim 1, wherein each hypercascade array comprises:
a first static barcode, wherein the first static barcode is selected from a library of at least about 10⁶ different first static barcode sequences; and/or
one or more second static barcodes, wherein each second static barcode is selected from a library of at least about 200 different second static barcode sequences.

27. (canceled)

28. (canceled)

29. The system of claim 1, wherein the editor is selected from the group comprising CRISPR-Cas9, base editors, prime editors, integrases, and recombinases.

30. The system of claim 1, wherein the editor is a base editor is capable of base editing the hypercascade unit, wherein said base editing comprises: adenine (A)-to-guanine (G) base editing and/or cytosine (C)-to-thymine (T) base editing.

31. The system of claim 30, wherein the base editor comprises:
saCas9-KKH, Cas9-VQR, Cas9-VRQR, Cas9-VRER, Cas9-NG, ABE7.7, pNMG-624, ABE3.2, ABE5.3, pNMG-558, pNMG-576, pNMG-577, pNMG-586, ABE7.2, pNMG-620, pNMG-617, pNMG-618, pNMG-620, pNMG-621, pNGM-622, pNMG-623, ABE6.3, ABE6.4, ABE7.8, ABE7.9, ABE7.10, ABEMax, ABE8e, CP1028-ABE8e, ABE7.10-CP1041, CP1041-ABE8e, or any combination thereof; and/or
an adenine base editor (ABE) and/or a cytosine base editor (CBE), wherein the ABE comprises monomer and dimer versions of one or more of ABE8e, ABE8e-V106W, SaABE8e, SaKKH-ABE8e, NG-ABE8e, ABE-xCas9, ABE8e-NRTH, ABE8e-NRRH, ABE8e-NRCH, ABE8e-NG-CP1041, ABE8e-VRQR-CP1041, ABE8e-CP1041, ABE8e-CP1028, ABE8e-VRQR, ABE8e-LbCas12a (LbABE8e), ABE8e-AsCas12a (enAsABE8e), ABE8e-SpyMac, ABE8e (TadA-8e V106W), ABE8e (K20A,R21A), and ABE8e (TadA-8e V82G).

32. (canceled)

33. (canceled)

34. The system of claim 1, wherein:
p is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50;
n is 2, 3, 4, 5, 6, 7, 8, 9, 10, 11, 12, 13, 14, or 15; and/or
m is 2, 3, 4, 5, 6, 7, 8, 9, 10, 11, 12, 13, 14, or 15.

35. (canceled)

36. (canceled)

37. (canceled)

38. The system of claim 1, wherein:
the length of the hypercascade array is at least, or most, about 200 bp, 400 bp, 600 bp, 800 bp, 1.0 kb, 1.5 kb, 2.0 kb, 2.5 kb, 3.0 kb, 3.5 kb, 4.0 kb, 4.5 kb, 5.0 kb, 5.5 kb, 6.0 kb, 6.5 kb, 7.0 kb, or 7.5 kb;
the system capable of linear editing for an increased period before saturation as compared to non-regenerative target arrays; and/or
the hypercascade array comprises an internal priming site capable of binding a custom sequencing primer, thereby enabling recovery of the entire hypercascade array along with the first and/or second static barcode(s) via a short-read sequencing protocol.

39. (canceled)

40. (canceled)

41. A nucleic acid composition, comprising:
one or more first polynucleotide(s) encoding the one or more hypercascade arrays of claim 1;
one or more second polynucleotide(s) encoding the n layer guide RNAs (gRNAs) of claim 1; and/or
one or more third polynucleotide(s) encoding the editor of claim 1.

42. (canceled)

43. (canceled)

44. (canceled)

45. (canceled)

46. (canceled)

47. A population of cells, comprising:
one or more hypercascade array(s) of claim 1;
the n layer guide RNAs (gRNAs) of claim 1; and/or
the editor of claim 1.

48. (canceled)

49. (canceled)

50. (canceled)

51. A method, comprising:
introducing the one or more hypercascade array(s) of claim 1, the n layer guide RNAs (gRNAs) of claim 1, and the editor of claim 1 into a cell or a first population of cells,
incubating the cell(s) for a period of time; and
obtaining sequence information of the hypercascade array(s) of each of the resulting second population of cells.

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)
